(19) United States
(12) Patent Application Publication
(10) Pub. No.: US 2024/0309012 A1
(43) Pub. Date: Sep. 19, 2024

(54) ISOQUINOLINE COMPOUND, MANUFACTURING METHOD THEREFOR, AND APPLICATION THEREOF

(71) Applicants: Shanghai Institute of Materia Medica, Chinese Academy of Sciences, Shanghai (CN); Wuhan Institute of Virology, Chinese Academy of Science, Wuhan, Hubei (CN)

(72) Inventors: Jingshan SHEN, Shanghai (CN); Zhaobing GAO, Shanghai (CN); Gengfu XIAO, Wuhan, Hubei (CN); Jia LI, Shanghai (CN); Feipu YANG, Shanghai (CN); Leike ZHANG, Wuhan, Hubei (CN); Bingqing XIA, Shanghai (CN); Xiangrui JIANG, Shanghai (CN); Yang HE, Shanghai (CN); Hualiang JIANG, Shanghai (CN)

(21) Appl. No.: 18/009,981

(22) PCT Filed: Jan. 11, 2021
(86) PCT No.: PCT/CN2021/071113

§ 371 Date: Jun. 12, 2023

(30) Foreign Application Priority Data
Jun. 12, 2020 (CN) 202010534755.5
Jun. 28, 2020 (CN) 202010754738.2
Oct. 30, 2020 (CN) 202011197404.6

(51) Int. Cl. C07D 491/18; A61K 31/4748; A61P 31/14; C07D 491/22; C07D 498/22; C07D 519/00

(52) U.S. Cl. CPC C07D 491/18; A61K 31/4748; A61P 31/14; C07D 491/22; C07D 498/22; C07D 519/00

(57) ABSTRACT

Provided are an isoquinoline compound represented by formula (I), a pharmaceutically acceptable salt thereof, an enantiomer thereof, a diastereomer, a racemate, a crystalline hydrate, and a solvate, as well as an application of a composition thereof in fighting a virus. R₁, R₂, R₃, and R₄ are as defined in the description. Further provided are a preparation method for the compound and uses thereof for preparing an inhibitor that inhibits a virus and/or a drug for prophylaxis and/or treatment of an illness related to a respiratory tract infection, pneumonia, etc. caused by a virus.

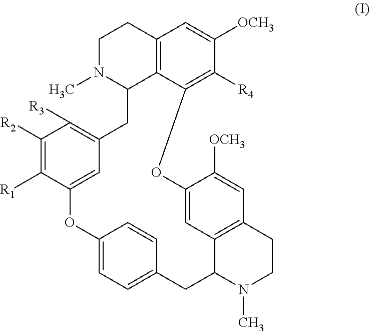

### TECHNICAL FIELD

The present invention relates to the field of medicinal chemistry and chemical synthesis. In particular, the present invention relates to isoquinoline compounds and preparation methods and applications thereof.

### BACKGROUND ART

In acute infectious diseases, most of them are viral infectious diseases, the incidence of viral infectious diseases is high, and the mortality rate is also high. Due to the limited means of detection and diagnosis, new outbreaks caused by new viruses are often sudden, random and unpredictable. Once an outbreak occurs, if there is no effective means of prevention and control, it is very easy to cause a large-scale epidemic and seriously threaten people's health and life safety.

The novel coronavirus (SARS-CoV-2) is the seventh coronavirus found by human beings at present. The novel coronavirus pneumonia (Corona Virus Disease 2019, COVID-19) is an acute infectious disease caused by this virus. At present, the virus has swept the world, and the number of infections and deaths has far exceeded that of SARS in 2003, which seriously threatens human health and social and economic development.

At present, there is no specific vaccine or antiviral drug for the severe pneumonia disease caused by the SARS-CoV-2 coronavirus. These infectious diseases have seriously affected people's life and health, and the development of effective antiviral drugs is imminent. It is of great social significance to develop low-toxicity and high-efficiency antiviral drugs for SARS-CoV-2 coronavirus to meet the clinical needs of patients with SARS-CoV-2 coronavirus infection at home and abroad.

The bis-benzyl isoquinoline compounds with good anti-inflammatory and immunomodulatory effects, such as berbamine and tetrandrine, have attracted the attention of those skilled in the art. From existing literature (Comparative analysis of antiviral efficacy of FDA-approved drugs against SARS-CoV-2 in human lung cells: Nafamostat is the most potent antiviral drug candidate.), it can be seen that berbamine's anti-novel coronavirus effect is not strong, and the anti-novel coronavirus activity of tetrandrine is stronger than that of berbamine, but the cytotoxicity is also greater, resulting in a low selectivity index/therapeutic index (SI), with potential safety hazards.

In summary, there is an urgent need in the art to develop novel bis-benzyl isoquinoline compounds with higher safety to inhibit SARS-CoV-2 coronavirus and to treat pneumonia caused by novel coronavirus infection.

### SUMMARY OF THE INVENTION

The purpose of the present invention is to provide a safer inhibitor for inhibiting the activity of viruses, especially SARS-CoV-2 coronaviruses, a preparation method thereof, and a new use thereof in the treatment, prevention and alleviation of diseases caused by virus infection, especially pneumonia caused by novel coronavirus infection.

In the first aspect of the present invention, it provides a bis-benzyl isoquinoline compound of formula I or a pharmaceutically acceptable salt thereof, or an enantiomer, a diastereomer, a racemate, a crystalline hydrate, a solvate thereof, or a mixture thereof,

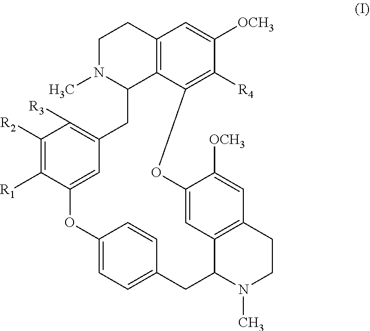

in the formula,
R₁, R₂, R₃ and R₄ are each independently selected from: hydrogen, halogen, nitro, mercapto, C₁˜C₆ alkoxy, halogenated C₁˜C₆ alkoxy, hydroxy C₁˜C₆ alkoxy, C₁˜C₆ alkylthio, C₁˜C₆ alkyl, halogenated C₁˜C₆ alkyl, amino C₁˜C₆ alkyl, hydroxy C₁˜C₆ alkyl, cyano C₁˜C₆ alkyl, C₃˜C₈ cycloalkyl, C₃˜C₈ cycloalkoxy, C₂˜C₆ alkenyl, C₂˜C₆ alkenyl carbonyl, C₂˜C₆ alkenyloxy, C₂˜C₆ alkynyl, C₂˜C₆ alkynyloxy, amino, amino substituted by C₁˜C₆ alkyl, amino substituted by benzyl, amino substituted by C₁˜C₆ alkanoyl, amino substituted by C₂˜C₆ alkenylacyl, cyano, C₁-C₆ carboxyl, C₁-C₆ aldehyde, C₁-C₆ alkanoyl, C₃-C₆ cycloalkyl acyl, halogenated C₁-C₆ alkanoyl, sulfonamino, sulfonamino substituted by C₁-C₆ alkyl, carbamoyl, phenyl carbamoyl, N-methyl-N-methoxyamino, carbamoyl substituted by C₁˜C₆ alkyl, carbamoyl substituted by C₃˜C₆ cycloalkyl, adamantylcarbamoyl, carbamoyl substituted by pyridinyl or pyrimidinyl, carbamoyl substituted by hydroxy C₁˜C₆ alkoxy C₁˜C₆ alkyl, C₁˜C₆ alkoxycarbonyl substituted by hydroxy C₁˜C₆ alkoxy, phenoxycarbonyl, hydroxymethyl substituted by C3˜C6 cycloalkyl, carboxyl C₁˜C₆ alkyl, C₁˜C₆ alkanesulfonyl, halogenated C₁˜C₆ alkanesulfonyl, amino C₁˜C₆ alkyl substituted by C₁˜C₆ alkyl, carbamoyloxy substituted by C₁˜C₆ alkyl, amino C₁˜C₆ alkyl substituted by C₁˜C₆ alkanoyl, C₁˜C₆ alkoxycarbonyl, carbamoyl C₁˜C₆ alkyl, carbamoyl C₁˜C₆ alkyl substituted by C₁˜C₆ alkyl, C₂˜C₆ alkenyl acyloxy (C₂˜C₆ alkenyl-CO—O—), C₂-C₁₀ ester,

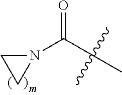

or —O—Z, where Z is

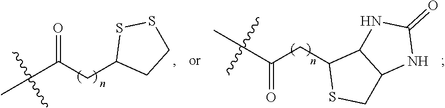

wherein n is 0, 1, 2, 3, or 4; m is 1, 2, 3, or 4;
wherein the phenylcarbamoyl, phenoxycarbonyl is optionally substituted with one or more substituents selected from F, Cl, Br, I, C₁˜C₆ alkyl, halogenated C₁˜C₆ alkyl, C₁˜C₆ alkoxy or halogenated C₁˜C₆ alkoxy;
or R₁ and R₂, or R₂ and R₃, together with the adjacent benzene ring, form benzo[5-6 membered monocyclic heterocycle] unsubstituted or substituted with 1-4 substituents, wherein the substituents are selected from halogen, hydroxy, mercapto, oxo, thio, C₁-C₆ alkyl, C₁-C₆ alkoxy, cyano; the heterocycle contains 1 to 3 heteroatoms selected from N, O, S.

In another preferred embodiment, the additional condition is that R₁ and R₄ are not both methoxy when both R₂ and R₃ are H.

In another preferred embodiment, the compound of formula I is a non-natural product.

In another preferred embodiment, the benzo[5-6 membered monocyclic heterocycle] is selected from the group consisting of

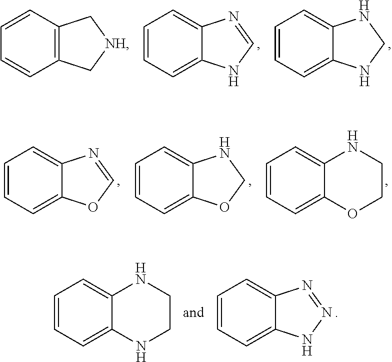

In another preferred embodiment, in the compound, R₁, R₂, R₃ and R₄ are each independently selected from hydrogen, halogen, nitro, mercapto, C₁-C₄ alkoxy, halogenated C₁-C₄ alkoxy, hydroxy C₁-C₄ alkoxy, C₁-C₄ alkylthio, C₁-C₄ alkyl, halogenated C₁-C₄ alkyl, amino C₁-C₄ alkyl, hydroxy C₁˜C₄ alkyl, cyano C₁˜C₄ alkyl, C₃˜C₆ cycloalkyl, C₃˜C₆ cycloalkoxy, C₂˜C₅ alkenyl, C₂˜C₅ alkenyl carbonyl, C₂˜C₅ alkenyloxy, C₂˜C₅ alkynyl, C₂˜C₅ alkynyloxy, amino, amino substituted by C₁˜C₄ alkyl, amino substituted by benzyl, amino substituted by C₁˜C₄ alkanoyl, amino substituted by C₂˜C₅ alkenylacyl, cyano, C₁˜C₄ carboxyl, C₁˜C₄ aldehyde, C₁˜C₄ alkanoyl, C₃˜C₅ cycloalkanoyl, halogenated C₁˜C₄ alkanoyl, sulfonylamino (—SO₂NH₂), sulfonylamino (—SO₂NH₂) substituted by C₁˜C₄ alkyl, carbamoyl (—CONH₂), phenylcarbamoyl, N-methyl-N-methoxyamino, carbamoyl substituted by C₁˜C₄ alkyl, carbamoyl substituted by C₃˜C₅ cycloalkyl, adamantyl carbamoyl, carbamoyl substituted by pyridinyl or pyrimidinyl, carbamoyl substituted by hydroxy C₁˜C₄ alkoxy C₁˜C₄ alkyl, C₁˜C₄ alkoxycarbonyl substituted by hydroxy C₁˜C₄ alkoxy, phenoxycarbonyl, hydroxymethyl substituted by C₃˜C₅ cycloalkyl, carboxyl C₁˜C₄ alkyl, C₁˜C₄ alkylsulfonyl, halogenated C₁˜C₄ alkylsulfonyl, amino C₁˜C₄ alkyl substituted by C₁˜C₄ alkyl, carbamoyloxy substituted by C₁˜C₄ alkyl, amino C₁˜C₄ alkyl substituted by C₁˜C₄ alkanoyl, C₁˜C₄ alkoxycarbonyl, carbamoyl C₁˜C₄ alkyl, carbamoyl C₁˜C₄ alkyl substituted by C₁˜C₄ alkyl, C₂˜C₅ alkenyl acyloxy (C₂˜C₅ alkenyl-CO—O—), C₂-C₆ ester,

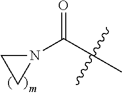

or —O—Z, wherein Z is

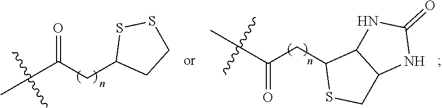

n is 0, 1, 2, 3, or 4; m is 1, 2, 3, or 4;

alternatively, R₁ and R₂, or R₂ and R₃, together with the adjacent benzene ring, may form benzo[5-6 membered monocyclic heterocycle] unsubstituted or substituted with 1 to 2 substituents; wherein the substituent is selected from halogen, hydroxy, mercapto, oxo(═O), thio(=S), C₁-C₆alkyl, C₁-C₆ alkoxy, cyano; wherein the heterocycle contains 1 to 3 heteroatoms selected from N, O, S.

In another preferred embodiment, in the compound, R₁, R₂, R₃ and R₄ are each independently selected from hydrogen, fluorine, chlorine, bromine, nitro, mercapto, methoxy, ethoxy, trifluoromethoxy, —SCH₃, —SCH₂CH₃, propyl, cyclopropyl, isopropyl, tert-butyl, trifluoromethyl, difluoromethoxy, bromomethyl, chloromethyl, vinyl, vinyl methyl, amino, N-methylamino, N-ethylamino, N,N-dimethylamino, N,N-diethylamino, cyano, carboxyl, aldehyde, —CH₂NH₂, —CH₂CH₂NH₂, —CH₂OH, —CH₂CH₂OH, —CH₂CN, —CH₂CH₂CN, formyl, acetyl, propionyl, trifluoroacetyl, sulfonylamino, carbamoyl, N-methylcarbamoyl, N,N-dimethylcarbamoyl, N-ethylcarbamoyl, N,N-diethylcarbamoyl, —CH₂CO₂H, —CH₂CH₂CO₂H, —SO₂CH₃, —SO₂CF₃, —CH₂NHMe, —CH₂NMe₂, —CH₂CONH₂, —NHCOCH₃, —CH₂NHCOCH₃, —CH₂CONHMe, —CH₂CONMe₂, or —O—Z, wherein Z is

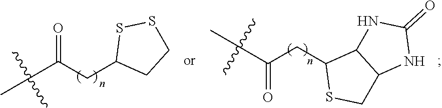

n is any integer from 0 to 4;
alternatively, R₁ and R₂, or R₂ and R₃, together with the adjacent benzene ring, may form benzo[5-6 membered monocyclic heterocycle] unsubstituted or substituted with 1-2 substituents selected from halogen, hydroxy, mercapto, oxo, thio, C₁-C₆ alkyl; the heterocycle contains 1 to 3 heteroatoms selected from N, O, S.

In another preferred embodiment, R₁ is hydrogen, fluorine, chlorine, bromine, mercapto, methoxy, ethoxy, trifluoromethoxy, —SCH₃, —SCH₂CH₃, propyl, cyclopropyl, isopropyl, tert-butyl, trifluoromethyl, difluoromethoxy, vinyl, vinyl methyl, amino, N-methylamino, N,N-dimethylamino, N,N-diethylamino, cyano, carboxyl, N-ethylamino, aldehyde, —CH₂NH₂, —CH₂CH₂NH₂, —CH₂OH, —CH₂CH₂OH, —CH₂CN, —CH₂CH₂CN, formyl, acetyl, propionyl, trifluoroacetyl, sulfonylamino, carbamoyl, N-methylcarbamoyl, N,N-dimethylcarbamoyl, N-ethylcarbamoyl, N,N-diethylcarbamoyl, —CH₂CO₂H, —CH₂CH₂CO₂H, —CH₂NHMe, —CH₂NMe₂, —CH₂CONH₂, —NHCOCH₃, —CH₂NHCOCH₃, —CH₂CONHMe or —CH₂CONMe₂.

In another preferred embodiment, R₂ is hydrogen, fluorine, chlorine, bromine, nitro, amino, N-methylamino, N-ethylamino, N,N-dimethylamino, N,N-diethylamino, sulfonylamino or —NHCOCH₃.

In another preferred embodiment, R₃ is hydrogen, fluorine, chlorine, bromine, nitro, amino, N-methylamino, N-ethylamino, N,N-dimethylamino, N,N-diethylamino, sulfonylamino or —NHCOCH₃.

In another preferred embodiment, R₄ is hydrogen, fluorine, chlorine, bromine, mercapto, methoxy, ethoxy, trifluoromethoxy, —SCH₃, —SCH₂CH₃, propyl, cyclopropyl, isopropyl, tert-butyl, trifluoromethyl, difluoromethoxy, vinyl, vinyl methyl, amino, N-methylamino, N-ethylamino, N,N-dimethylamino, N,N-diethylamino, cyano, carboxyl, aldehyde, —CH₂NH₂, —CH₂CH₂NH₂, —CH₂OH, —CH₂CH₂OH, —CH₂CN, —CH₂CH₂CN, formyl, acetyl, propionyl, trifluoroacetyl, sulfonylamino, carbamoyl, N-methylcarbamoyl, N,N-dimethylcarbamoyl, N-ethylcarbamoyl, N, N-diethylcarbamoyl, —CH₂CO₂H, —CH₂CH₂CO₂H, —CH₂NHMe, —CH₂NMe₂, —CH₂CONH₂, —NHCOCH₃, —CH₂NHCOCH₃, —CH₂CONHMe or —CH₂CONMe₂.

In another preferred embodiment, the compound of formula I is chiral or achiral.

In another preferred embodiment, the compound has the structure shown in formula I-a:

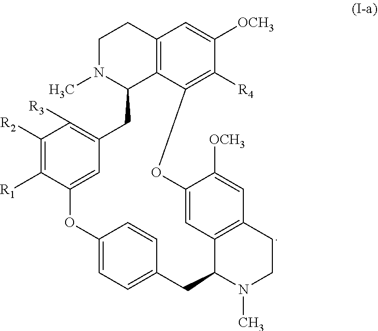

In another preferred embodiment, the compound has the structure shown in formula I-b, I-c or I-d:

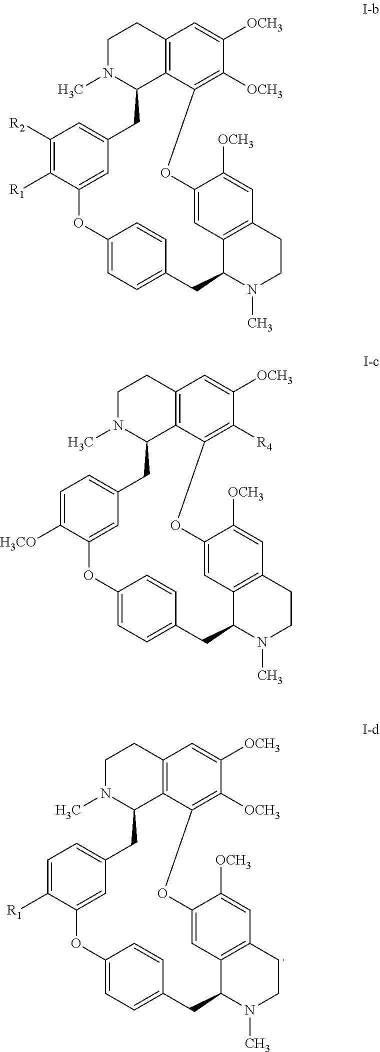

In another preferred embodiment, the compound of formula (I) is selected from the following compounds:

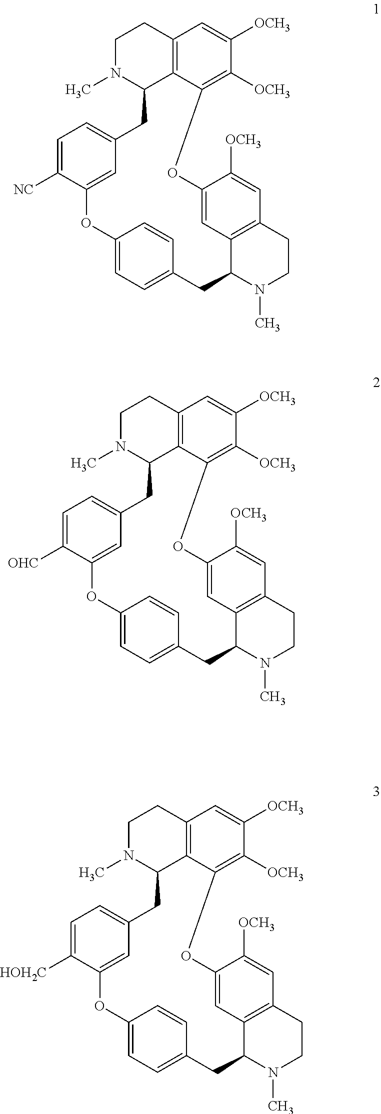 [SMILES: COc1cc2c3cc1Oc1c(OC)c(OC)cc4c1[C@@H](Cc1ccc(C#N)c(c1)Oc1ccc(cc1)C[C@@H]3N(C)CC2)N(C)CC4 ; COc1cc2c3cc1Oc1c(OC)c(OC)cc4c1[C@@H](Cc1ccc(C=O)c(c1)Oc1ccc(cc1)C[C@@H]3N(C)CC2)N(C)CC4 ; COc1cc2c3cc1Oc1c(OC)c(OC)cc4c1[C@@H](Cc1ccc(CO)c(c1)Oc1ccc(cc1)C[C@@H]3N(C)CC2)N(C)CC4]

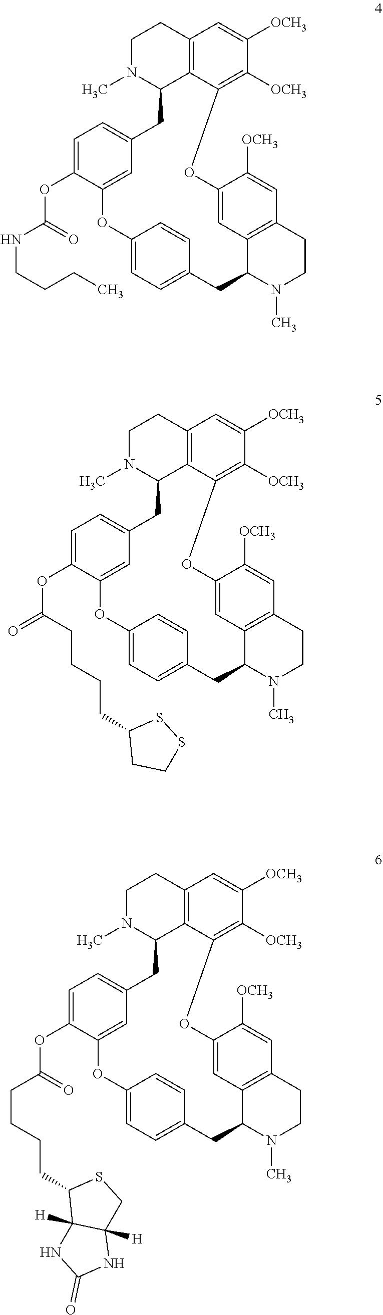 [SMILES: CCCCNC(=O)Oc1ccc2cc1Oc1ccc(cc1)C[C@H]1c3cc(c(OC)cc3CCN1C)Oc1c(OC)c(OC)cc3c1[C@@H](C2)N(C)CC3 ; COc1cc2c3cc1Oc1c(OC)c(OC)cc4c1[C@@H](Cc1ccc(OC(=O)CCCC[C@H]5CCSS5)c(c1)Oc1ccc(cc1)C[C@@H]3N(C)CC2)N(C)CC4 ; COc1cc2c3cc1Oc1c(OC)c(OC)cc4c1[C@@H](Cc1ccc(OC(=O)CCCC[C@@H]5SC[C@@H]6NC(=O)N[C@@H]65)c(c1)Oc1ccc(cc1)C[C@@H]3N(C)CC2)N(C)CC4]

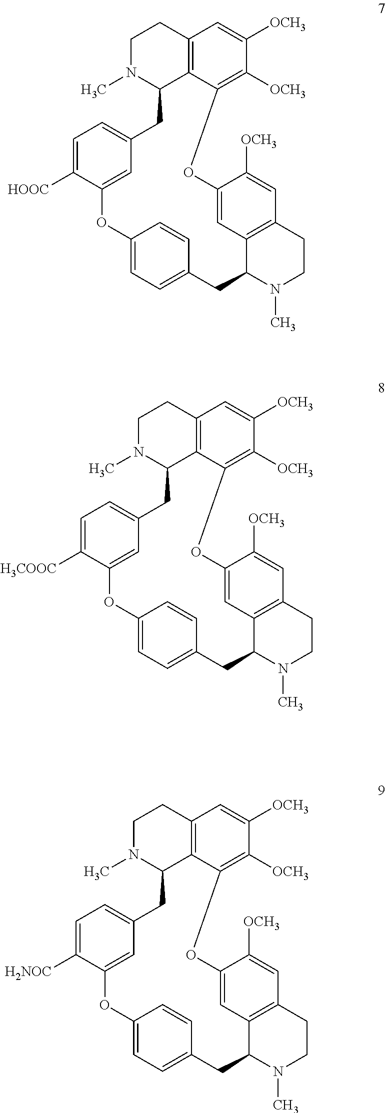

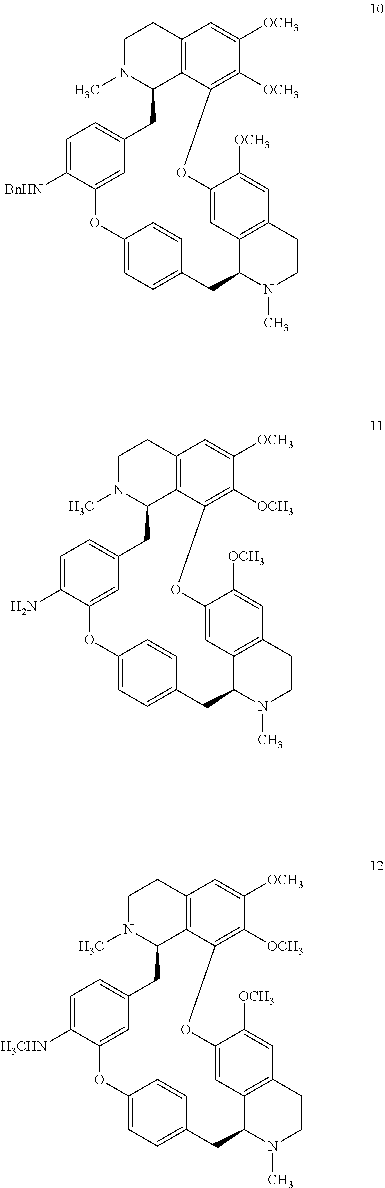

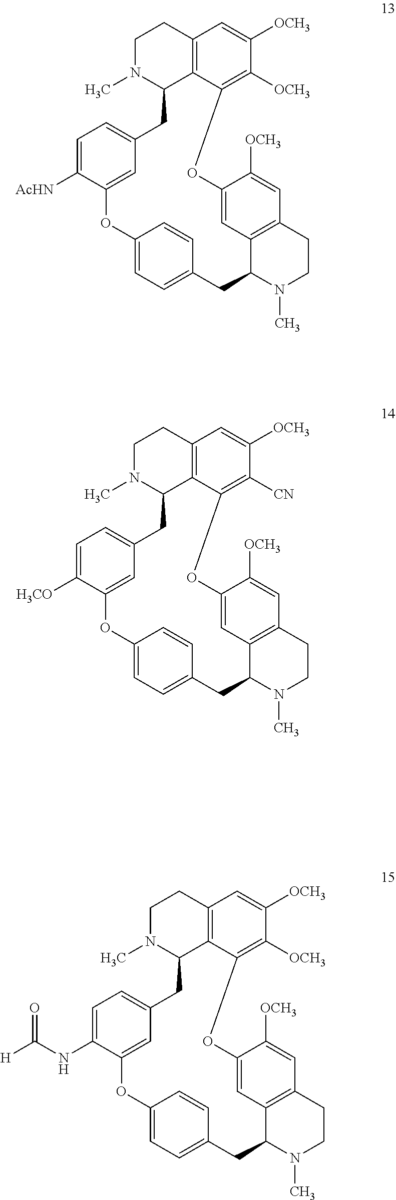 [SMILES: COc1cc2c3cc1Oc1c(OC)c(OC)cc4c1[C@@H](Cc1ccc(NC(C)=O)c(c1)Oc1ccc(cc1)C[C@@H]3N(C)CC2)N(C)CC4 ; COc1ccc2cc1Oc1ccc(cc1)C[C@H]1c3cc(c(OC)cc3CCN1C)Oc1c(C#N)c(OC)cc3c1[C@@H](C2)N(C)CC3 ; COc1cc2c3cc1Oc1c(OC)c(OC)cc4c1[C@@H](Cc1ccc(NC=O)c(c1)Oc1ccc(cc1)C[C@@H]3N(C)CC2)N(C)CC4]

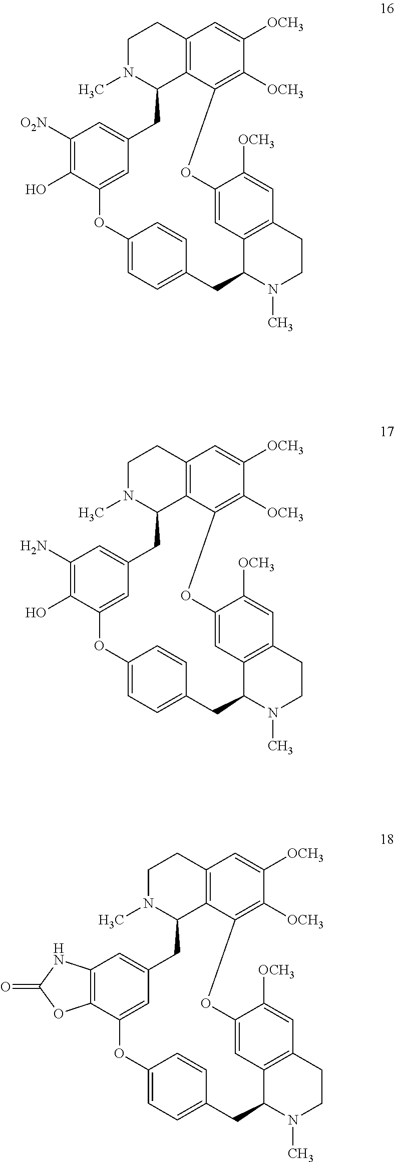 [SMILES: COc1cc2c3cc1Oc1c(OC)c(OC)cc4c1[C@@H](Cc1cc(c(O)c([N+](=O)[O-])c1)Oc1ccc(cc1)C[C@@H]3N(C)CC2)N(C)CC4 ; COc1cc2c3cc1Oc1c(OC)c(OC)cc4c1[C@@H](Cc1cc(N)c(O)c(c1)Oc1ccc(cc1)C[C@@H]3N(C)CC2)N(C)CC4 ; COc1cc2c3cc1Oc1c(OC)c(OC)cc4c1[C@@H](Cc1cc(c5oc(=O)[nH]c5c1)Oc1ccc(cc1)C[C@@H]3N(C)CC2)N(C)CC4]

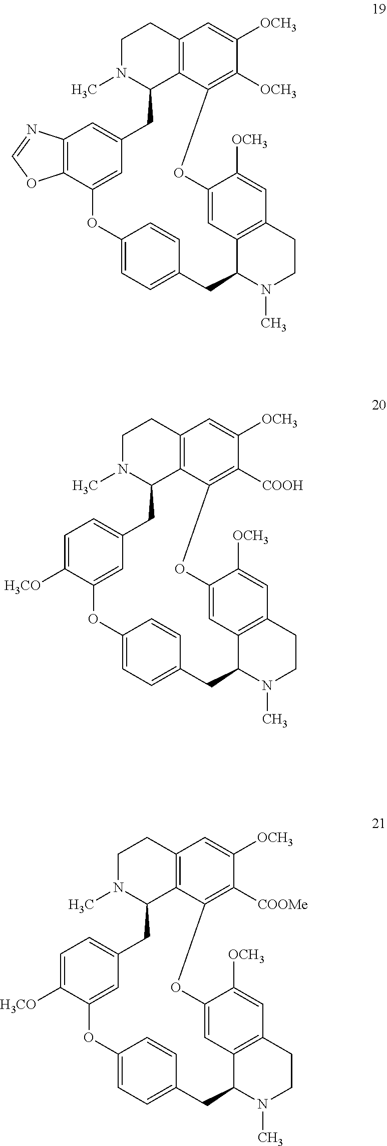 [SMILES: COc1cc2c3cc1Oc1c(OC)c(OC)cc4c1[C@@H](Cc1cc(c5ocnc5c1)Oc1ccc(cc1)C[C@@H]3N(C)CC2)N(C)CC4 ; COc1ccc2cc1Oc1ccc(cc1)C[C@H]1c3cc(c(OC)cc3CCN1C)Oc1c(C(=O)O)c(OC)cc3c1[C@@H](C2)N(C)CC3 ; COC(=O)c1c(OC)cc2c3c1Oc1cc4c(cc1OC)CCN(C)[C@H]4Cc1ccc(cc1)Oc1cc(ccc1OC)C[C@H]3N(C)CC2]

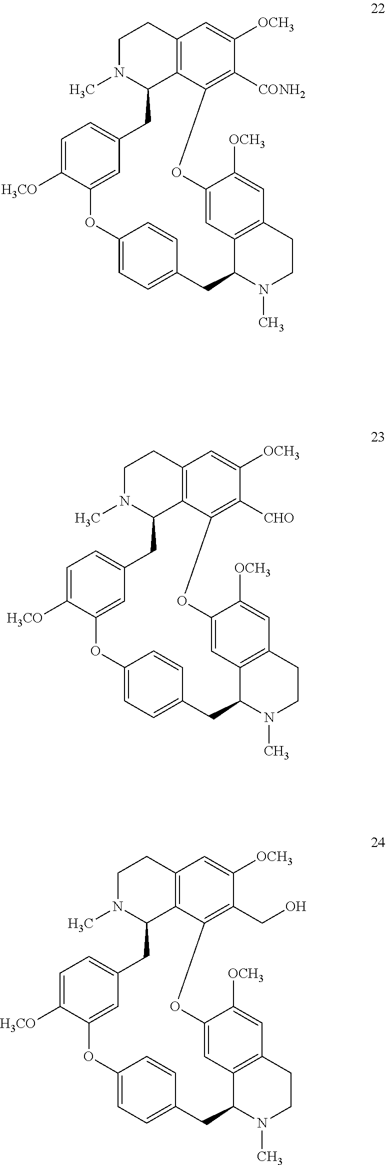 [SMILES: COc1ccc2cc1Oc1ccc(cc1)C[C@H]1c3cc(c(OC)cc3CCN1C)Oc1c(C(N)=O)c(OC)cc3c1[C@@H](C2)N(C)CC3 ; COc1ccc2cc1Oc1ccc(cc1)C[C@H]1c3cc(c(OC)cc3CCN1C)Oc1c(C=O)c(OC)cc3c1[C@@H](C2)N(C)CC3 ; COc1ccc2cc1Oc1ccc(cc1)C[C@H]1c3cc(c(OC)cc3CCN1C)Oc1c(CO)c(OC)cc3c1[C@@H](C2)N(C)CC3]

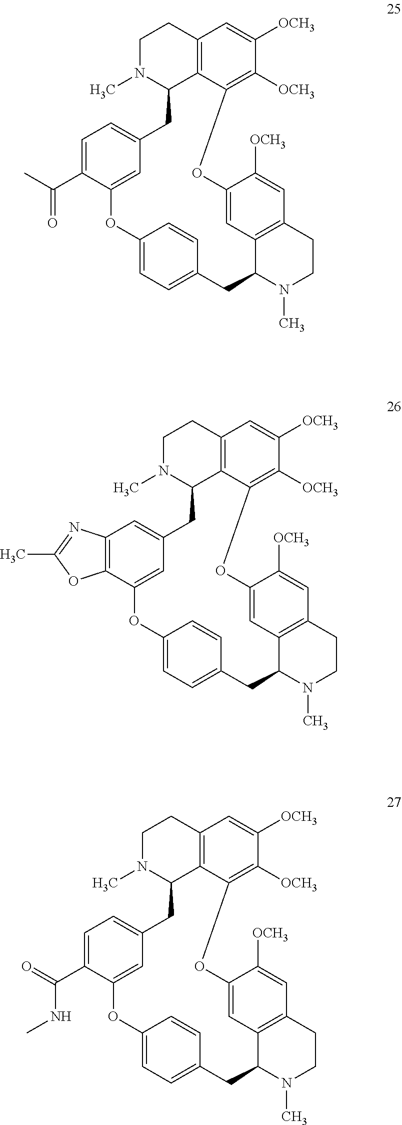 [SMILES: COc1cc2c3cc1Oc1c(OC)c(OC)cc4c1[C@@H](Cc1ccc(C(C)=O)c(c1)Oc1ccc(cc1)C[C@@H]3N(C)CC2)N(C)CC4 ; COc1cc2c3cc1Oc1c(OC)c(OC)cc4c1[C@@H](Cc1cc(c5oc(C)nc5c1)Oc1ccc(cc1)C[C@@H]3N(C)CC2)N(C)CC4 ; CNC(=O)c1ccc2cc1Oc1ccc(cc1)C[C@H]1c3cc(c(OC)cc3CCN1C)Oc1c(OC)c(OC)cc3c1[C@@H](C2)N(C)CC3]

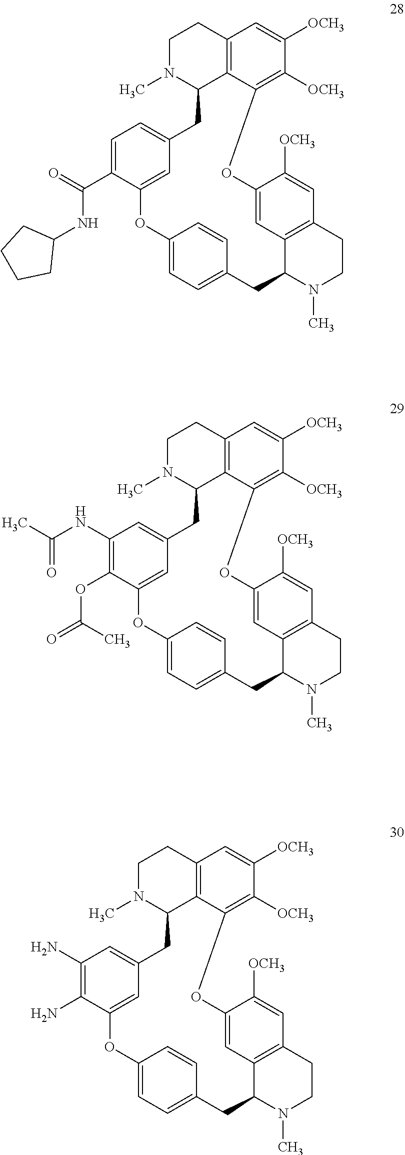 [SMILES: COc1cc2c3cc1Oc1c(OC)c(OC)cc4c1[C@@H](Cc1ccc(C(=O)NC5CCCC5)c(c1)Oc1ccc(cc1)C[C@@H]3N(C)CC2)N(C)CC4 ; COc1cc2c3cc1Oc1c(OC)c(OC)cc4c1[C@@H](Cc1cc(NC(C)=O)c(OC(C)=O)c(c1)Oc1ccc(cc1)C[C@@H]3N(C)CC2)N(C)CC4 ; COc1cc2c3cc1Oc1c(OC)c(OC)cc4c1[C@@H](Cc1cc(N)c(N)c(c1)Oc1ccc(cc1)C[C@@H]3N(C)CC2)N(C)CC4]

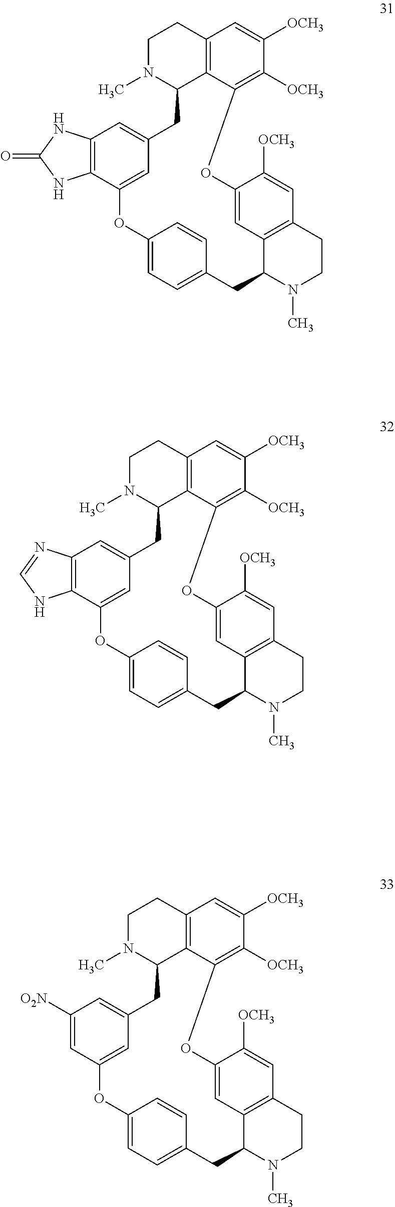 [SMILES: COc1cc2c3cc1Oc1c(OC)c(OC)cc4c1[C@@H](Cc1cc(c5[nH]c(=O)[nH]c5c1)Oc1ccc(cc1)C[C@@H]3N(C)CC2)N(C)CC4 ; COc1cc2c3cc1Oc1c(OC)c(OC)cc4c1[C@@H](Cc1cc(c5[nH]cnc5c1)Oc1ccc(cc1)C[C@@H]3N(C)CC2)N(C)CC4 ; COc1cc2c3cc1Oc1c(OC)c(OC)cc4c1[C@@H](Cc1cc(cc([N+](=O)[O-])c1)Oc1ccc(cc1)C[C@@H]3N(C)CC2)N(C)CC4]

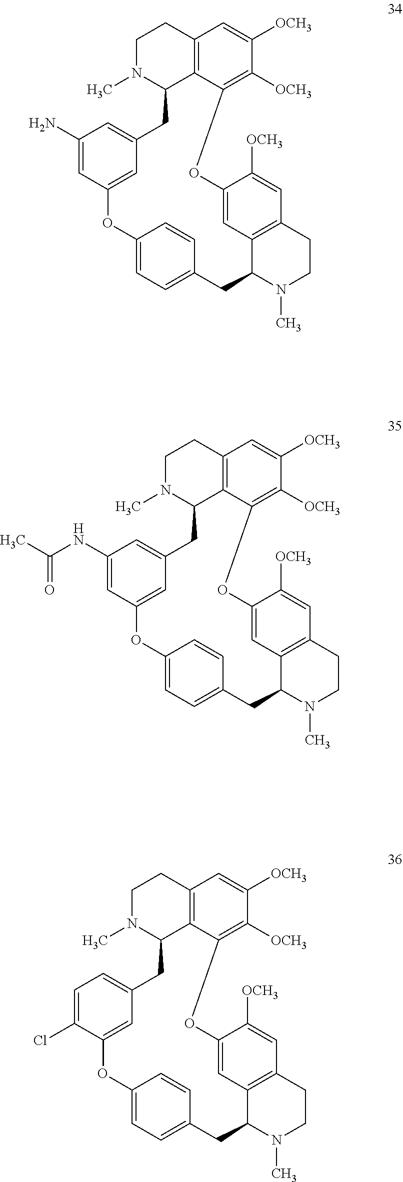 [SMILES: COc1cc2c3cc1Oc1c(OC)c(OC)cc4c1[C@@H](Cc1cc(N)cc(c1)Oc1ccc(cc1)C[C@@H]3N(C)CC2)N(C)CC4 ; COc1cc2c3cc1Oc1c(OC)c(OC)cc4c1[C@@H](Cc1cc(NC(C)=O)cc(c1)Oc1ccc(cc1)C[C@@H]3N(C)CC2)N(C)CC4 ; COc1cc2c3cc1Oc1c(OC)c(OC)cc4c1[C@@H](Cc1ccc(Cl)c(c1)Oc1ccc(cc1)C[C@@H]3N(C)CC2)N(C)CC4]

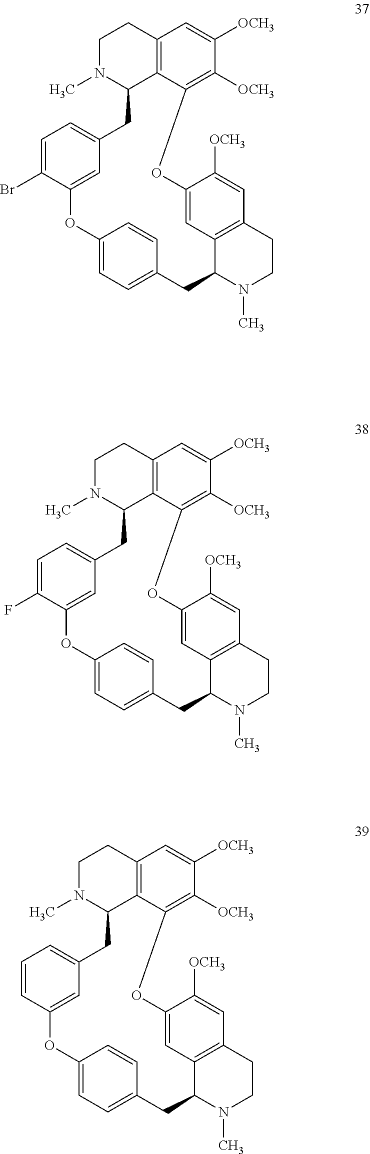 [SMILES: COc1cc2c3cc1Oc1c(OC)c(OC)cc4c1[C@@H](Cc1ccc(Br)c(c1)Oc1ccc(cc1)C[C@@H]3N(C)CC2)N(C)CC4 ; COc1cc2c3cc1Oc1c(OC)c(OC)cc4c1[C@@H](Cc1ccc(F)c(c1)Oc1ccc(cc1)C[C@@H]3N(C)CC2)N(C)CC4 ; COc1cc2c3cc1Oc1c(OC)c(OC)cc4c1[C@@H](Cc1cccc(c1)Oc1ccc(cc1)C[C@@H]3N(C)CC2)N(C)CC4]

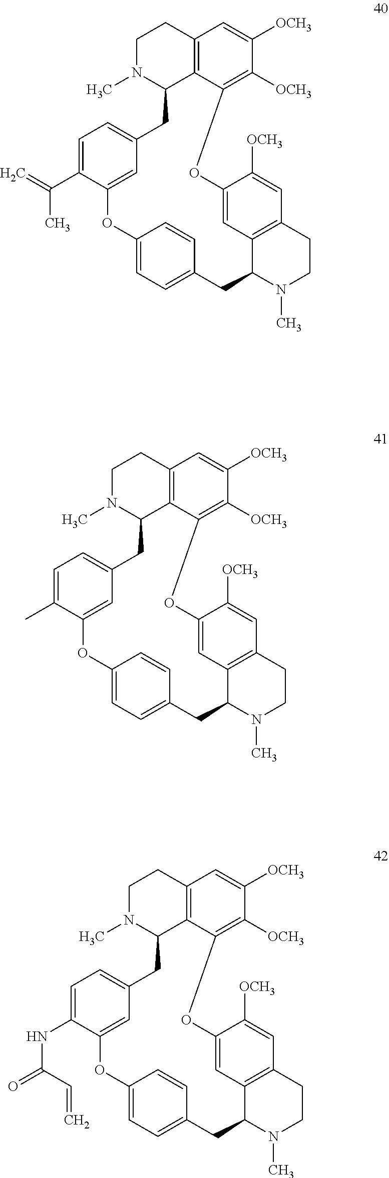 [SMILES: C=C(C)c1ccc2cc1Oc1ccc(cc1)C[C@H]1c3cc(c(OC)cc3CCN1C)Oc1c(OC)c(OC)cc3c1[C@@H](C2)N(C)CC3 ; COc1cc2c3cc1Oc1c(OC)c(OC)cc4c1[C@@H](Cc1ccc(C)c(c1)Oc1ccc(cc1)C[C@@H]3N(C)CC2)N(C)CC4 ; C=CC(=O)Nc1ccc2cc1Oc1ccc(cc1)C[C@H]1c3cc(c(OC)cc3CCN1C)Oc1c(OC)c(OC)cc3c1[C@@H](C2)N(C)CC3]

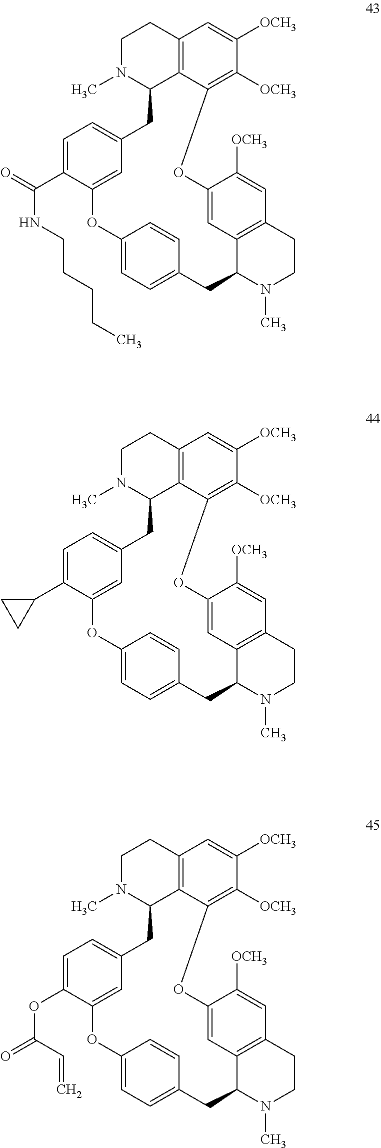 [SMILES: CCCCCNC(=O)c1ccc2cc1Oc1ccc(cc1)C[C@H]1c3cc(c(OC)cc3CCN1C)Oc1c(OC)c(OC)cc3c1[C@@H](C2)N(C)CC3 ; COc1cc2c3cc1Oc1c(OC)c(OC)cc4c1[C@@H](Cc1ccc(C5CC5)c(c1)Oc1ccc(cc1)C[C@@H]3N(C)CC2)N(C)CC4 ; C=CC(=O)Oc1ccc2cc1Oc1ccc(cc1)C[C@H]1c3cc(c(OC)cc3CCN1C)Oc1c(OC)c(OC)cc3c1[C@@H](C2)N(C)CC3]

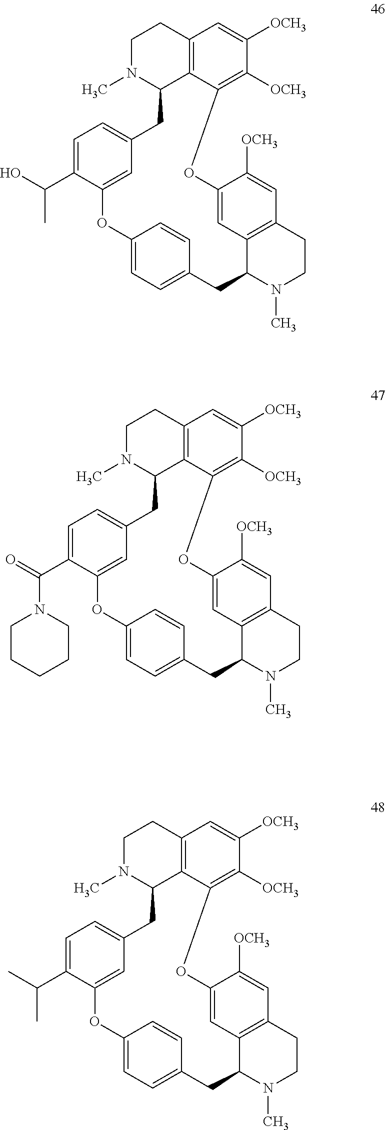 [SMILES: COc1cc2c3cc1Oc1c(OC)c(OC)cc4c1[C@@H](Cc1ccc(C(C)O)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)N5CCCCC5)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(C)C)c(c1)Oc1ccc(cc1)C[C@@H]3N(C)CC2)N(C)CC4]

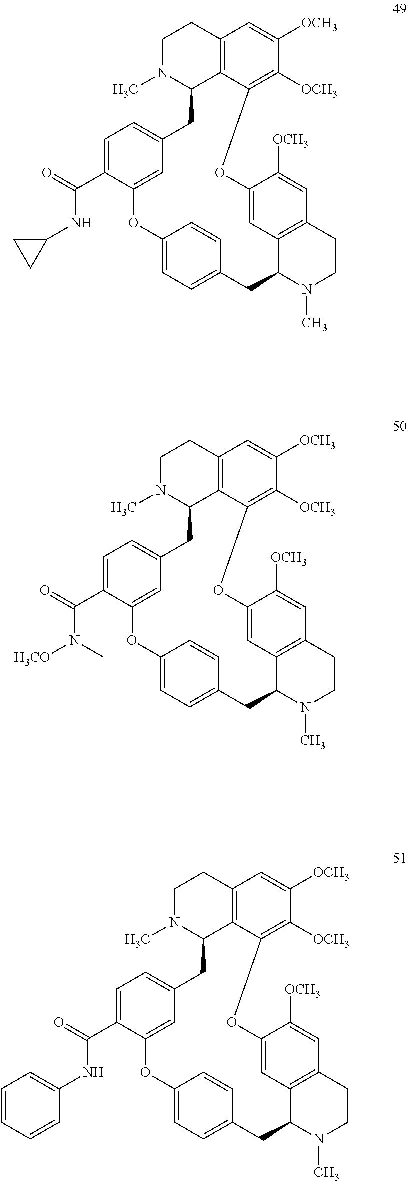 [SMILES: COc1cc2c3cc1Oc1c(OC)c(OC)cc4c1[C@@H](Cc1ccc(C(=O)NC5CC5)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)N(C)OC)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)Nc5ccccc5)c(c1)Oc1ccc(cc1)C[C@@H]3N(C)CC2)N(C)CC4]

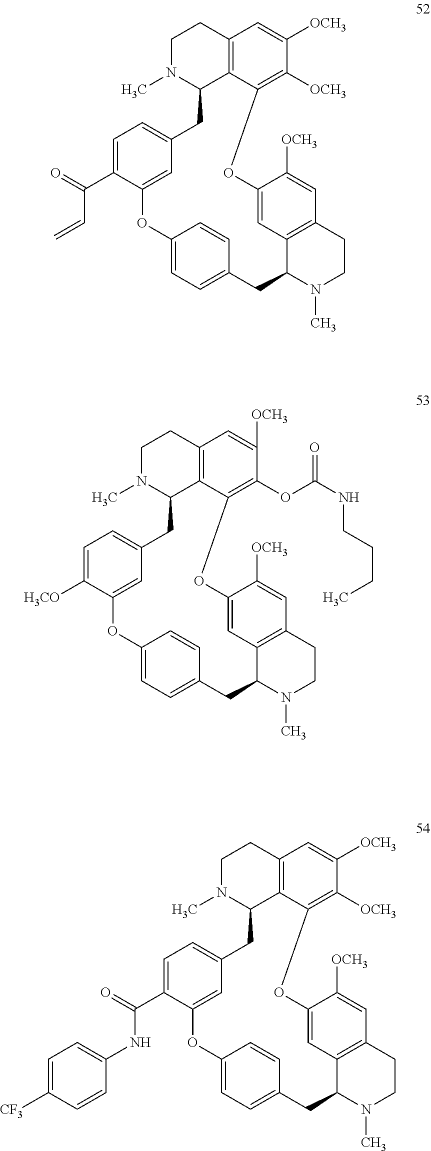 [SMILES: C=CC(=O)c1ccc2cc1Oc1ccc(cc1)C[C@H]1c3cc(c(OC)cc3CCN1C)Oc1c(OC)c(OC)cc3c1[C@@H](C2)N(C)CC3 ; CCCCNC(=O)Oc1c(OC)cc2c3c1Oc1cc4c(cc1OC)CCN(C)[C@H]4Cc1ccc(cc1)Oc1cc(ccc1OC)C[C@H]3N(C)CC2 ; COc1cc2c3cc1Oc1c(OC)c(OC)cc4c1[C@@H](Cc1ccc(C(=O)Nc5ccc(C(F)(F)F)cc5)c(c1)Oc1ccc(cc1)C[C@@H]3N(C)CC2)N(C)CC4]

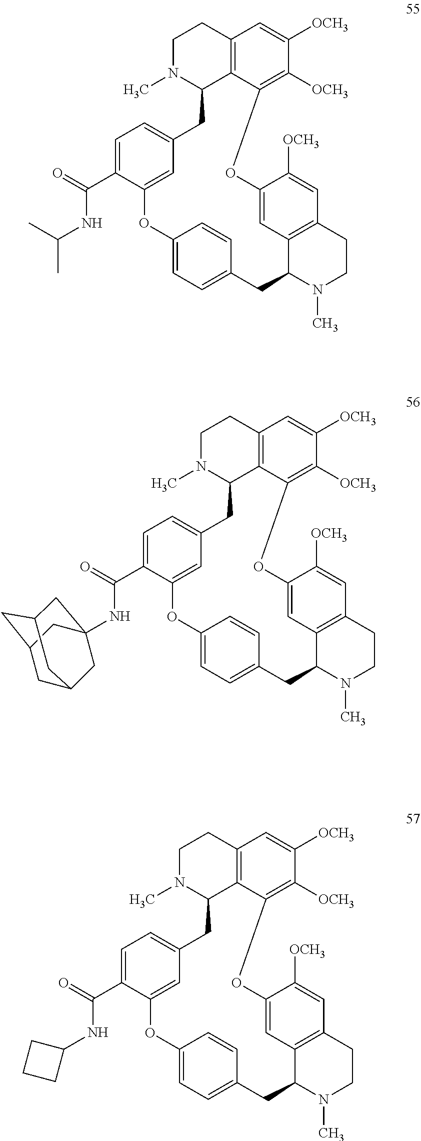 [SMILES: COc1cc2c3cc1Oc1c(OC)c(OC)cc4c1[C@@H](Cc1ccc(C(=O)NC(C)C)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)NC56CC7CC(CC(C7)C5)C6)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)NC5CCC5)c(c1)Oc1ccc(cc1)C[C@@H]3N(C)CC2)N(C)CC4]

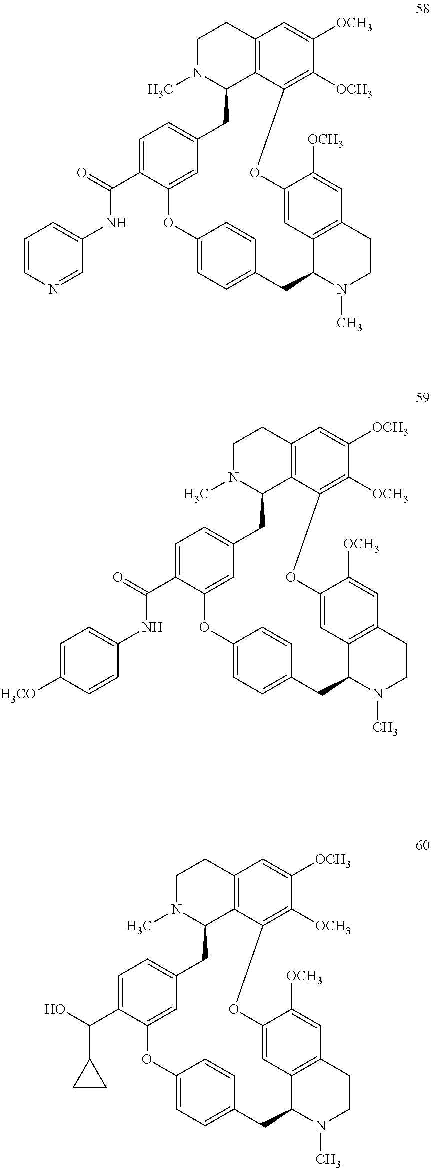 [SMILES: COc1cc2c3cc1Oc1c(OC)c(OC)cc4c1[C@@H](Cc1ccc(C(=O)Nc5cccnc5)c(c1)Oc1ccc(cc1)C[C@@H]3N(C)CC2)N(C)CC4 ; COc1ccc(NC(=O)c2ccc3cc2Oc2ccc(cc2)C[C@H]2c4cc(c(OC)cc4CCN2C)Oc2c(OC)c(OC)cc4c2[C@@H](C3)N(C)CC4)cc1 ; COc1cc2c3cc1Oc1c(OC)c(OC)cc4c1[C@@H](Cc1ccc(C(O)C5CC5)c(c1)Oc1ccc(cc1)C[C@@H]3N(C)CC2)N(C)CC4]

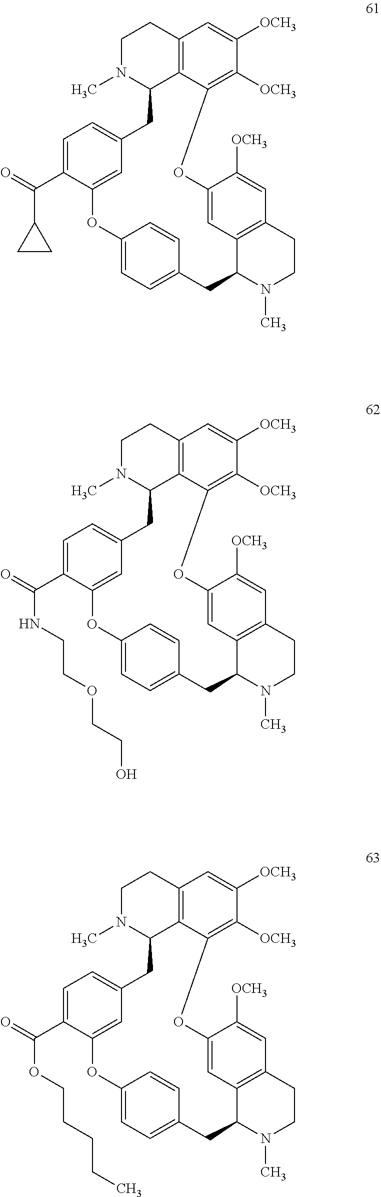 [SMILES: COc1cc2c3cc1Oc1c(OC)c(OC)cc4c1[C@@H](Cc1ccc(C(=O)C5CC5)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)NCCOCCO)c(c1)Oc1ccc(cc1)C[C@@H]3N(C)CC2)N(C)CC4 ; CCCCCOC(=O)c1ccc2cc1Oc1ccc(cc1)C[C@H]1c3cc(c(OC)cc3CCN1C)Oc1c(OC)c(OC)cc3c1[C@@H](C2)N(C)CC3]

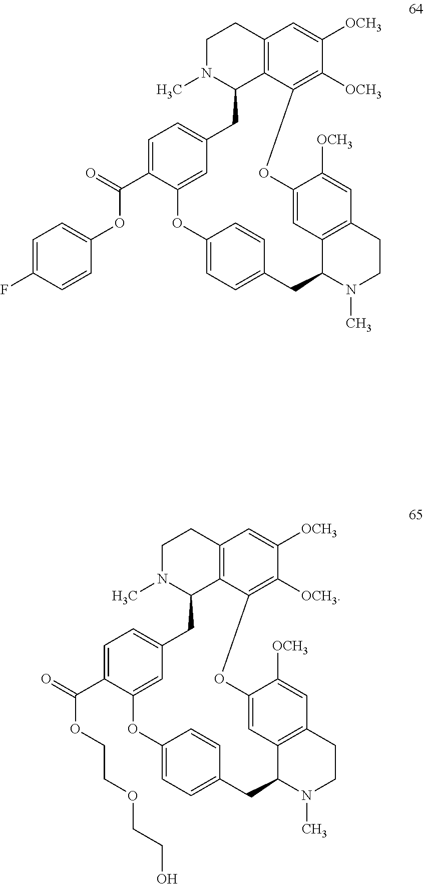 [SMILES: COc1cc2c3cc1Oc1c(OC)c(OC)cc4c1[C@@H](Cc1ccc(C(=O)Oc5ccc(F)cc5)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)OCCOCCO)c(c1)Oc1ccc(cc1)C[C@@H]3N(C)CC2)N(C)CC4]

In another preferred embodiment, the compound of formula (I) is selected from the following compounds:

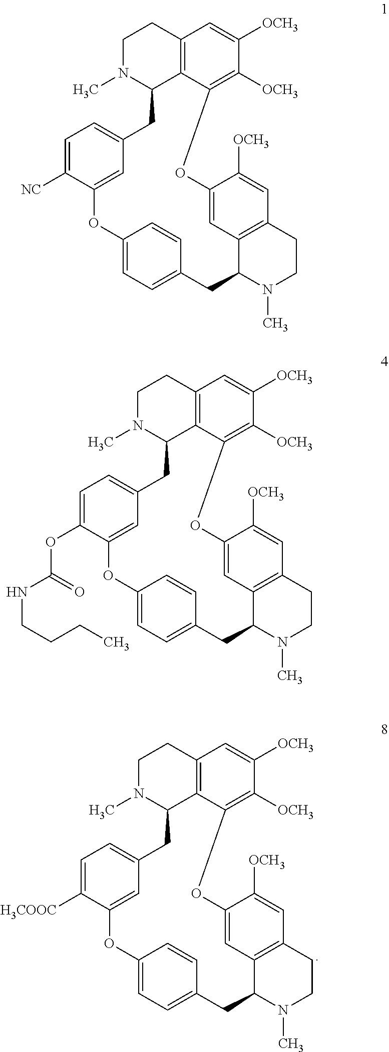

In the second aspect of the present invention, it provides a method for preparing the bis-benzyl isoquinoline compound of the first aspect, and the method is selected from the group consisting of:
a) the bis-benzyl isoquinoline compound is obtained by alkylation reaction between a phenolic hydroxyl-containing bis-benzyl isoquinoline as a raw material and an alkylation reagent;
b) the bis-benzyl isoquinoline compound is obtained by an acylation reaction between a phenolic hydroxy-containing bis-benzyl isoquinoline as a raw material and an acylation reagent;
c) compound (I-1b) is obtained by reacting berbamine as a raw material with sulfonylation reagent in the presence of a base;
the bis-benzyl isoquinoline compound 1-1 is obtained by coupling (I-1b) and coupling reagents, and the reaction is shown in scheme 1:

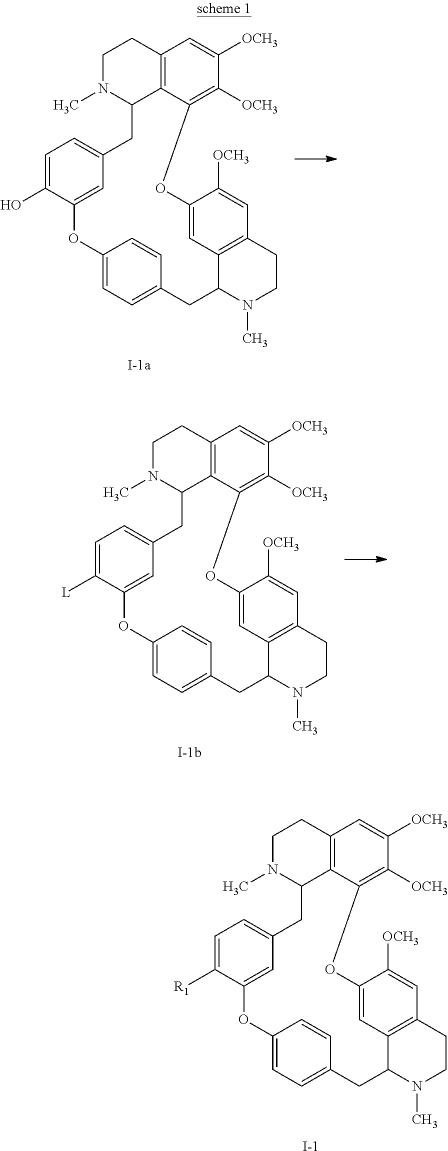

in formula I-1, R₁ is selected from hydrogen, halogen, C₁-C₆ alkylthio, C₁-C₆ alkyl, C₃-C₆ cycloalkyl, C₂-C₆ alkenyl, C₂-C₆ alkynyl, amino substituted by C₁-C₆ alkyl, amino substituted by benzyl, cyano, carboxy, aldehyde, C₁-C₆ alkanoyl;
L in formula 1-1b is selected from a leaving group;
d) using berbamine as a raw material, the compound of formula (I-2) is prepared by nitration, reduction and ring-closing reaction, as shown in the following scheme 2:

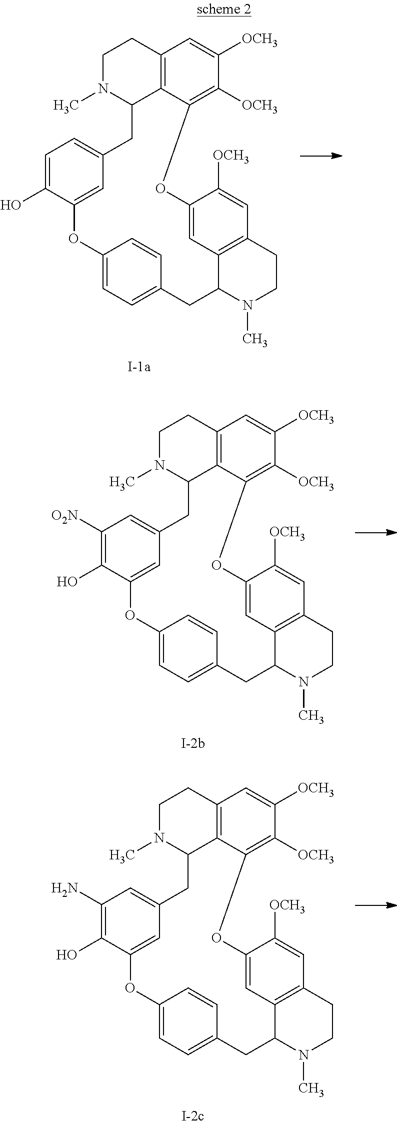

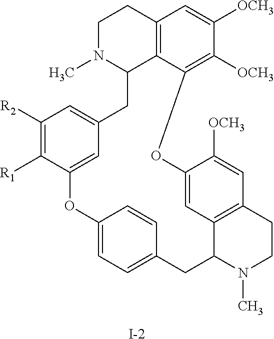

in formula I-2, R₁ and R₂, together with the adjacent benzene ring, form benzo[5-6 membered monocyclic heterocycle] unsubstituted or substituted with 1-4 substituents;
e) using berbamine as a raw material, the compound of formula (I-3) is prepared by multi-step reaction, as shown in scheme 3:

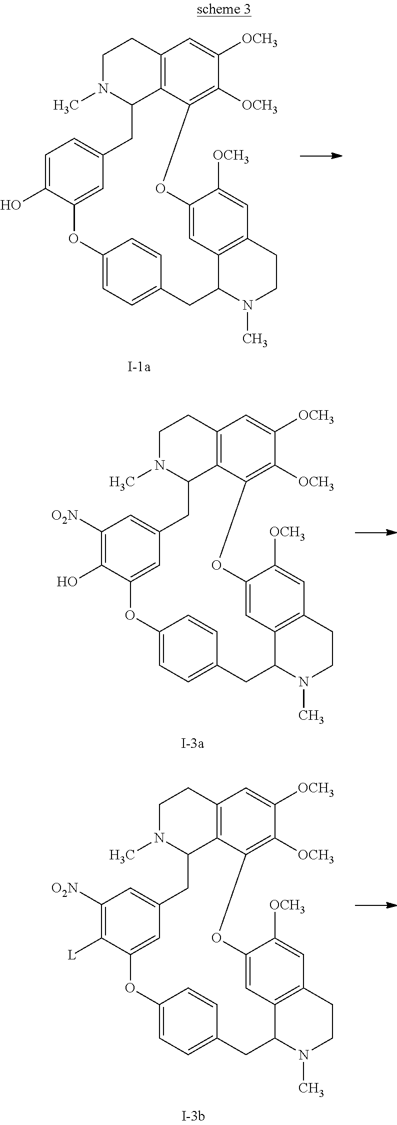

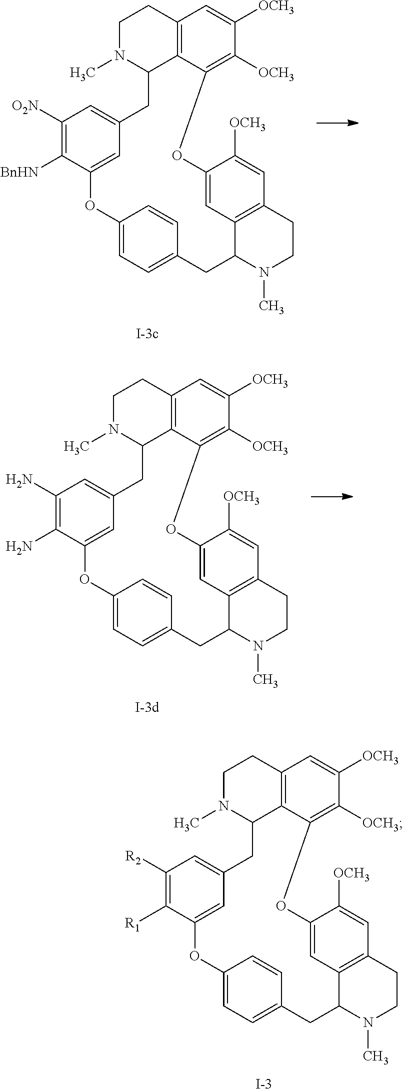

in formula I-3, R₁ and R₂ together with the adjacent benzene ring form benzo[5-6 membered monocyclic heterocycle] unsubstituted or substituted with 1-4 substituents;
f) using Fangchinoline as a raw material, reacting with sulfonylation reagent in the presence of a base to obtain compound (I-4b); and conducting a coupling reaction between compound (I-4b) and coupling reagent to obtain a compound of formula I-4, as shown in scheme 4:

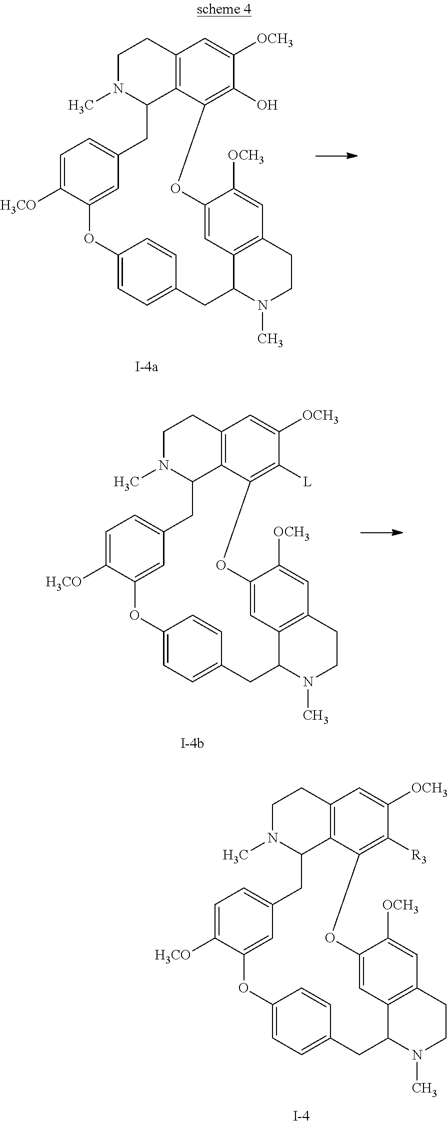

in formula I-4, R₃ is selected from hydrogen, halogen, C₁-C₆ alkylthio, C₁-C₆ alkyl, C₃-C₆ cycloalkyl, C₂-C₆ alkenyl, C₂-C₆ alkynyl, amino substituted by C₁-C₆ alkyl, amino substituted by benzyl, cyano, carboxy, aldehyde, C₁-C₆ alkanoyl;
L in formula I-4b is a leaving group;
g) the compounds obtained by methods a) to f) are subjected to functional group transformation to obtain the bis-benzyl isoquinoline compounds.

In another preferred embodiment, the method is that using berbamine as a raw material, undergoing a condensation acylation reaction with a carboxylic acid to obtain the bis-benzyl isoquinoline compound.

In another preferred embodiment, the condensation acylation reaction is carried out in the presence of a condensing agent selected from the group consisting of N,N′-dicyclohexylcarbodiimide (DCC), 1-ethyl-3-(3-dimethylaminopropyl) carbodiimide (EDCI), O-benzotriazole-N,N,N′,N′-tetramethylurea tetrafluoroborate (TBTU), and a combination thereof.

In another preferred embodiment, the leaving group L is selected from the group consisting of: C₁-C₆ alkyl sulfonyloxy, halogenated C₁-C₆ alkyl sulfonyloxy, benzenesulfonyloxy, naphthalenesulfonyloxy, and a combination thereof, preferably, L is methylsulfonyloxy or trifluoromethylsulfonyloxy.

In another preferred embodiment, the sulfonylation reagent is selected from the group consisting of C₁˜C₆ alkyl sulfonyl chloride, C₁˜C₆ alkyl sulfonic anhydride, benzenesulfonyl chloride, benzenesulfonic anhydride, naphthalene sulfonyl chloride, naphthalene sulfonic anhydride, preferably methylsulfonyl chloride, methanesulfonic anhydride, trifluoromethanesulfonyl chloride and trifluoromethanesulfonic anhydride.

In another preferred embodiment, the solvent is selected from the group consisting of dichloromethane, tetrahydrofuran, N,N-dimethylformamide, methanol, ethanol, acetonitrile, toluene, acetone, dioxane and chloroform.

In another preferred embodiment, the base is selected from inorganic base or organic base.

In another preferred embodiment, the inorganic base is selected from sodium hydroxide, potassium hydroxide, cesium hydroxide, barium hydroxide, potassium hydride, sodium hydride, sodium tert-butoxide, potassium tert-butoxide, potassium carbonate, sodium carbonate or calcium carbonate; and the organic base is selected from pyridine, triethylamine, diisopropylethylamine, N,N-dimethylaniline or N,N-lutidine.

In another preferred embodiment, the coupling reaction is carried out in the presence of a palladium catalyst and a base.

In another preferred embodiment, the palladium catalyst is selected from: palladium acetate (Pd(OAc)₂), bis (triphenylphosphine) palladium dichloride ((Ph₃P)₂PdCl₂), bis(benzonitrile) palladium chloride ((PhCN)₂PdCl₂), tetrakis (triphenylphosphine) palladium (Pd(PPh₃)₄), bis (triphenylphosphine) palladium acetate ((Ph₃P)₂Pd(Oac)₂), 1,2-bis (diphenylphosphino) ethane palladium dichloride ((PdCl₂(dppe)₂)), bis (1, 2-bis (diphenylphosphino) ethane) palladium (Pd(dppe)₂), bis (dibenzylideneacetone) palladium (Pd(dba)₂), tris (dibenzylideneacetone) dipalladium (Pd₂(dba)₃), [1,3-bis (diphenylphosphino) propane] palladium dichloride (PdCl₂(dippp)) and [1,1′-bis (diphenylphosphino) ferrocene] palladium dichloride (Pd(dppf)Cl₂), or a combination thereof.

In another preferred embodiment, scheme 1 may further include other reaction solvents that do not interfere with the reaction.

In another preferred embodiment, a suitable ligand can also be added as a reaction promoter in scheme 1.

In another preferred embodiment, the suitable ligand is selected from: 2,2′-diphenylphosphino-1,1′-binaphthyl (BINAP), tri-tert-butyl (P(t-Bu)₃), 1′-bis-(diphenylphosphino) ferrocene (dppf), 2-dicyclohexylphospho-2,4,6-triisopropylbiphenyl (x-phos), 4,5-bis-diphenylphosphine-9,9-dimethylxanthene (Xantphos), tri-tert-butylphosphine tetrafluoroborate, and tris (2-methylphenyl) phosphine (P(o-tolyl)₃), or a combination thereof. In another preferred embodiment, the coupling reagent is selected from the group consisting of C₁-C₆ alkyl boronic acid, cyclopropyl boronic acid, benzylamine, potassium cyanide, zinc cyanide, tributyl vinyl tin, CO, CO₂, formic acid, sodium formate, lithium formate, C₁-C₆ alkyl thiolate sodium, sodium methanesulfonate, or a combination thereof.

In another preferred embodiment, the benzo[5-6 membered monocyclic heterocycle] is selected from:

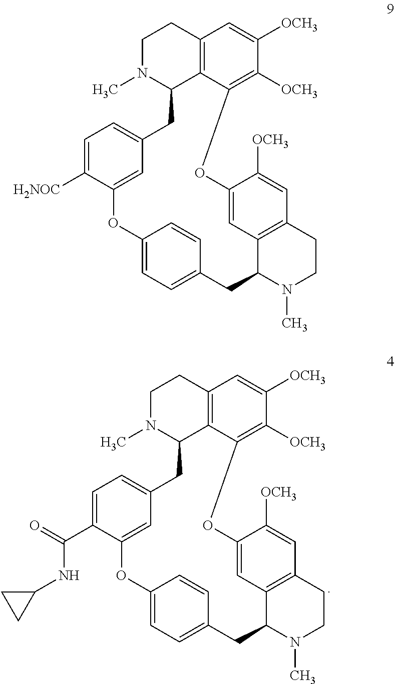 [SMILES: COc1cc2c3cc1Oc1c(OC)c(OC)cc4c1[C@@H](Cc1ccc(C(N)=O)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)NC5CC5)c(c1)Oc1ccc(cc1)C[C@@H]3N(C)CC2)N(C)CC4]

In another preferred embodiment, for benzo[5-6 membered monocyclic heterocycle], the substituent is selected from halogen, hydroxy, mercapto, amino, oxo (═O), thio (═S), C₁-C₆ alkyl; and the heterocycle contains 1 to 3 heteroatoms selected from N, O and S.

In another preferred embodiment, the nitration reagent in the nitration reaction is selected from the group consisting of nitric acid, a mixture of concentrated sulfuric acid and nitric acid, a mixture of nitric acid, sodium nitrate and concentrated sulfuric acid, a mixture of potassium nitrate and concentrated sulfuric acid, a mixture of sodium nitrite and concentrated sulfuric acid, and a mixture of acetic acid and nitric acid.

In another preferred embodiment, the nitration reagent in the nitration reaction is a mixture of acetic acid and nitric acid.

In another preferred embodiment, the mixing ratio of the mixture is not limited.

In another preferred embodiment, the temperature of the nitration reaction is −20° C.˜room temperature.

In another preferred embodiment, the reduction reaction uses Pd/C as a catalyst.

In another preferred embodiment, the reduction reaction uses hydrogen, ammonium formate or formic acid as a reducing agent.

In another preferred embodiment, the reduction reaction is carried out in a lower alcohol or a lower alcohol-water mixed solvent.

In another preferred embodiment, the lower alcohol is selected from: methanol, ethanol, isopropanol or a combination thereof.

In another preferred embodiment, the reduction reaction is carried out in the range of 0-100° C., preferably 0-40° C.

In another preferred embodiment, the reduction reaction is carried out under normal pressure.

In another preferred embodiment, the reduction reaction hydrogenates for 1-10h.

In another preferred embodiment, the ring-closing reaction is carried out in the presence of a ring-closing reagent.

In another preferred embodiment, the ring-closing reagent includes: phosgene, triphosgene, 1,1′-carbonyldiimidazole (CDI), urea, carbon tetrabromide, formic acid, trimethyl orthoformate, trimethyl orthoacetate, triethyl orthoacetate, acetyl chloride, chloroacetyl chloride, bromoacetyl bromide, bromoacetyl chloride, or a combination thereof.

In another preferred embodiment, the ring-closing reaction is carried out in the presence or absence of a base.

In another preferred embodiment, the base is selected from: inorganic base or organic base; wherein the inorganic base is selected from sodium hydroxide, potassium hydroxide, potassium hydride, sodium hydride, sodium tert-butoxide, potassium tert-butoxide, potassium carbonate, sodium carbonate, cesium carbonate and sodium bicarbonate; wherein the organic base is selected from pyridine, triethylamine, diisopropylethylamine, N,N-dimethylaniline and N,N-dimethylpyridin.

In another preferred embodiment, scheme 2 may further include other reaction solvents that do not interfere with the reaction.

In another preferred embodiment, for benzo[5-6 membered monocyclic heterocycle], the substituent is selected from halogen, hydroxy, mercapto, amino, oxo (═O), thio (═S), C₁-C₆ alkyl; and the heterocycle contains 1 to 3 heteroatoms selected from N, O and S.

In another preferred embodiment, the benzo[5-6 membered monocyclic heterocycle] is selected from:

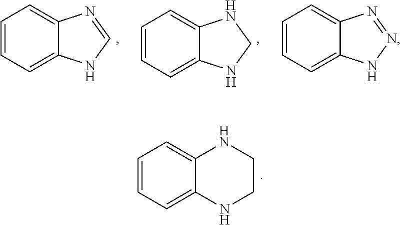

In the formula I-3b, L is a leaving group.

In another preferred embodiment, the leaving group L is selected from the group consisting of: C₁-C₆ alkylsulfonyloxy, halogenated C₁-C₆ alkylsulfonyloxy, benzenesulfonyloxy, naphthalenesulfonyloxy; preferably methylsulfonyloxy and trifluoromethylsulfonyloxy.

In another preferred embodiment, the method e) includes the following steps:
e1) the compound of formula (I-1a) and the nitration reagent undergo an ortho-nitration reaction to generate the compound of formula (I-3a);
e2) the compound of formula (I-3a) is reacted with a sulfonylation reagent in the presence of a base in a suitable solvent to obtain the compound of formula (1-3b);
e3) the compound of formula (I-3b) and benzylamine undergo a Buchwald-Hartig reaction to obtain the compound of formula (I-3c);
e4) the compound of formula (I-3c) undergoes a reduction reaction to obtain the compound of formula (I-3d);
e5) the compound of formula (1-3d) undergoes a ring-closing reaction to obtain the compound of formula (I-3).

In another preferred embodiment, the nitration reagent in step e1) is a mixture of concentrated sulfuric acid and nitric acid, a mixture of nitric acid, sodium nitrate and concentrated sulfuric acid, a mixture of potassium nitrate and concentrated sulfuric acid, a mixture of sodium nitrite and concentrated sulfuric acid, a mixture of acetic acid and nitric acid, preferably a mixture of acetic acid and nitric acid.

In another preferred embodiment, the mixing ratio of the mixture is not limited.

In another preferred embodiment, the temperature of the nitration reaction is −20° C.˜room temperature.

In another preferred embodiment, the time of the nitration reaction is 10 minutes to 12 hours.

In another preferred embodiment, the sulfonylation reagent in the step e2) is selected from: C₁˜C₆ alkyl sulfonyl chloride, C₁˜C₆ alkyl sulfonic anhydride, benzenesulfonyl chloride, benzenesulfonic anhydride, naphthalene sulfonyl chloride, naphthalene sulfonic anhydride, preferably methylsulfonyl chloride, methanesulfonic anhydride, trifluoromethanesulfonyl chloride and trifluoromethanesulfonic anhydride.

In another preferred embodiment, the solvent in step e2) is selected from the group consisting of dichloromethane, tetrahydrofuran, N,N-dimethylformamide, methanol, ethanol, acetonitrile, toluene, acetone, dioxane and chloroform.

In another preferred embodiment, the base in step e2) is selected from inorganic base or organic base; wherein the inorganic base is selected from sodium hydroxide, potassium hydroxide, cesium hydroxide, barium hydroxide, potassium hydride, sodium hydride, sodium tert-butoxide, potassium tert-butoxide, potassium carbonate, sodium carbonate or calcium carbonate; wherein the organic base is selected 1 pyridine, triethylamine, diisopropylethylamine, N,N-dimethylaniline and N,N-dimethylpyridine.

In another preferred embodiment, the coupling reaction in step e3) is carried out in the presence of a palladium catalyst and a base.

In another preferred embodiment, the palladium catalyst is selected from: palladium acetate (Pd(OAc)₂), bis (triphenylphosphine) palladium dichloride ((Ph₃P)₂PdCl₂), bis(benzonitrile) palladium chloride ((PhCN)₂PdCl₂), tetrakis (triphenylphosphine) palladium (Pd(PPh₃)₄), bis (triphenylphosphine) palladium acetate ((Ph₃P)₂Pd(OAc)₂), 1,2-bis (diphenylphosphino) ethane palladium dichloride ((PdCl₂(dppe)₂)), bis (1, 2-bis (diphenylphosphino) ethane) palladium (Pd(dppe)₂), bis (dibenzylideneacetone) palladium (Pd(dba)₂), tris (dibenzylideneacetone) dipalladium (Pd₂(dba)₃), [1,3-bis (diphenylphosphino) propane] palladium dichloride (PdCl₂(dippp)) and [1,1′-bis (diphenylphosphino) ferrocene] palladium dichloride (Pd(dppf)C₁₂), or a combination thereof.

In another preferred embodiment, the base is selected from sodium bis (trimethylsilyl) amide, potassium tert-butoxide, sodium tert-butoxide, cesium carbonate, potassium phosphate, sodium phosphate, sodium methoxide, sodium ethoxide, potassium hydroxide, sodium hydroxide, potassium fluoride, sodium fluoride, tetrabutylammonium fluoride (TBAF), sodium acetate, potassium acetate, cesium carbonate, potassium carbonate and sodium carbonate, or a combination thereof.

In another preferred embodiment, the reaction may further comprise other reaction solvents that do not interfere with the reaction.

In another preferred embodiment, the reaction in step e3) may further include a suitable ligand as a reaction promoter to carry out the above reaction.

In another preferred embodiment, the suitable ligand is selected from the group consisting of 2,2′-diphenylphosphino-1,1′-binaphthyl (BINAP), tri-tert-butyl (P(t-Bu)₃), 1,1′-bis-(diphenylphosphino) ferrocene (dppf), 2-dicyclohexylphospho-2,4,6-triisopropylbiphenyl (x-phos), 4,5-bis-diphenylphosphine-9,9-dimethylxanthene (Xantphos), tri-tert-butylphosphine tetrafluoroborate or tris (2-methylphenyl) phosphine (P(o-tolyl)₃).

In another preferred embodiment, the reduction reaction in step e4) uses Pd/C, palladium hydroxide or palladium hydroxide on carbon as a catalyst.

In another preferred embodiment, the reduction reaction in step e4) uses ammonium formate or hydrogen as a reducing agent.

In another preferred embodiment, the reduction reaction is carried out in a lower alcohol or a lower alcohol-water mixed solvent.

In another preferred embodiment, the lower alcohol is selected from: methanol, ethanol, isopropanol or a combination thereof.

In another preferred embodiment, the reduction reaction is carried out in 20˜100° C. range.

In another preferred embodiment, the reduction reaction is carried out under normal pressure.

In another preferred embodiment, the ring-closing reaction in step e5) is carried out in the presence of a ring-closing reagent.

In another preferred embodiment, the ring-closing reagent is selected from the group consisting of phosgene, triphosgene, 1,1′-carbonyldiimidazole (CDI), urea, formic acid, sodium nitrite, carbon disulfide, thiophosgene, triethyl orthoacetate, chloroacetic acid, bromoacetic acid, ethyl bromoacetate, methyl bromoacetate, chloroacetamide, or a combination thereof.

In another preferred embodiment, the ring-closing reaction is carried out in the presence or absence of a base.

In another preferred embodiment, the base is selected from: inorganic base or organic base.

In another preferred embodiment, the inorganic base is selected from sodium hydroxide, potassium hydroxide, potassium hydride, sodium hydride, sodium tert-butoxide, potassium tert-butoxide, potassium carbonate, sodium carbonate, cesium carbonate or sodium bicarbonate; wherein the organic base is selected from pyridine, triethylamine, diisopropylethylamine, N,N-dimethylaniline and N,N-dimethylpyridine.

In another preferred embodiment, step e5) may further comprise other reaction solvents that do not interfere with the reaction.

In another preferred embodiment, the leaving group L is selected from the group consisting of C₁-C₆ alkylsulfonyloxy, halogenated C₁-C₆ alkylsulfonyloxy, benzenesulfonyloxy, and naphthalenesulfonyloxy; preferably methanesulfonyloxy and trifluoromethanesulfonyloxy.

In another preferred embodiment, the sulfonylation reagent is selected from the group consisting of C₁˜C₆ alkyl sulfonyl chloride, C₁˜C₆ alkyl sulfonic anhydride, benzenesulfonyl chloride, benzenesulfonic anhydride, naphthalene sulfonyl chloride, naphthalene sulfonic anhydride, preferably methylsulfonyl chloride, methanesulfonic anhydride, trifluoromethanesulfonyl chloride and trifluoromethanesulfonic anhydride. The solvent is selected from the group of dichloromethane, consisting tetrahydrofuran, N,N-dimethylformamide, methanol, ethanol, acetonitrile, toluene, acetone, dioxane and chloroform.

In another preferred embodiment, the base is selected from inorganic base or organic base.

In another preferred embodiment, the inorganic base is selected from sodium hydroxide, potassium hydroxide, cesium hydroxide, barium hydroxide, potassium hydride, sodium hydride, sodium tert-butoxide, potassium tert-butoxide, potassium carbonate, sodium carbonate and calcium carbonate; and the organic base is selected from pyridine, triethylamine, diisopropylethylamine, N,N-dimethylaniline and N, N-lutidine.

In another preferred embodiment, the coupling reaction is carried out in the presence of a palladium catalyst and a base.

In another preferred embodiment, the palladium catalyst is selected from the group consisting of: palladium acetate (Pd(OAc)₂), bis (triphenylphosphine) palladium dichloride ((Ph₃P)₂PdCl₂), bis(benzonitrile) palladium chloride ((PhCN)₂PdCl₂), tetrakis (triphenylphosphine) palladium (Pd(PPh₃)₄), bis (triphenylphosphine) palladium acetate ((Ph₃P)₂Pd(OAc)₂), 1,2-bis (diphenylphosphino) ethane palladium dichloride ((PdCl₂(dppe)₂)), bis (1, 2-bis (diphenylphosphino) ethane) palladium (Pd(dppe)₂), bis (dibenzylideneacetone) palladium (Pd(dba)₂), tris (dibenzylideneacetone) dipalladium (Pd₂(dba)₃), [1,3-bis (diphenylphosphino) propane] palladium (PdCl₂(dippp)), dichloride [1,1′-bis (diphenylphosphino) ferrocene] palladium dichloride (Pd(dppf)C₁₂), or a combination thereof.

In another preferred embodiment, the base is selected from sodium bis (trimethylsilyl) amide, potassium tert-butoxide, sodium tert-butoxide, cesium carbonate, potassium phosphate, sodium phosphate, sodium methoxide, sodium ethoxide, potassium hydroxide, sodium hydroxide, potassium fluoride, sodium fluoride, tetrabutylammonium fluoride (TBAF), sodium acetate, potassium acetate, cesium carbonate, potassium carbonate, sodium carbonate, or a combination thereof.

In another preferred embodiment, the scheme 4 may further include other reaction solvents that do not interfere with the reaction.

In another preferred embodiment, in the scheme 4, a suitable ligand can also be added as a reaction promoter to carry out the above reaction.

In another preferred embodiment, the suitable ligand is selected from: tri-tert-butyl (P(t-Bu)₃), 2,2′-diphenylphosphino-1,1′-binaphthyl (BINAP), (dppf), ferrocene 1,1′-bis-(diphenylphosphino) (x-phos), 2-dicyclohexylphospho-2,4,6-triisopropylbiphenyl 4,5-bis-diphenylphosphine-9,9-dimethylxanthene (Xantphos), tri-tert-butylphosphine tetrafluoroborate, and tris (2-methylphenyl) phosphine (P(o-tolyl)₃). The coupling reagents include, but are not limited to, C₁-C₆ alkyl boronic acid, cyclopropyl boronic acid, benzylamine, potassium cyanide, zinc cyanide, tributylvinyl tin, CO, CO₂, formic acid, sodium formate, lithium formate, C₁-C₆ alkyl thiolate sodium, sodium methanesulfonate.

In another preferred embodiment, the functional group conversion reaction includes: oxidative hydrolysis reaction, hydroboration-oxidation reaction, condensation acylation reaction, reduction reaction, acylation reaction, esterification reaction, Grignard reaction, chlorination reaction or bromination reaction.

In another preferred embodiment, the oxidative hydrolysis reaction is carried out in the presence of an oxidizing agent and a base.

In another preferred embodiment, the oxidative hydrolysis system includes: hydrogen peroxide/sodium hydroxide, hydrogen peroxide/potassium hydroxide, hydrogen peroxide/potassium carbonate, and hydrogen peroxide/sodium carbonate.

In another preferred embodiment, the hydroboration-oxidation reaction is that the olefin group is first added with a boron reagent, and then it was oxidized and hydrolyzed to an alcohol.

In another preferred embodiment, the boron reagent includes: borane, 9-BBN.

In another preferred embodiment, the condensation acylation reaction is carried out in the presence of a condensing agent.

In another preferred embodiment, the condensing agent includes: N, N′-dicyclohexylcarbodiimide (DCC), 1-ethyl-3-(3-dimethylaminopropyl) carbodiimide (EDCI), O-benzotriazole-N,N, N′,N′-tetramethylurea tetrafluoroborate (TBTU).

In another preferred embodiment, the reduction reaction is carried out in the presence of a reducing agent.

In another preferred embodiment, the reducing agent includes: hydrogen, ammonium formate, sodium borohydride, potassium borohydride, diisobutylaluminum hydride (DIBAL), borane.

In another preferred embodiment, the acylation reaction is carried out in the presence of an acylating reagent.

In another preferred embodiment, the acylating reagent includes: acetyl chloride, acetic anhydride, propionyl chloride, propionic anhydride, methanesulfonyl chloride and the like.

In another preferred embodiment, the esterification reaction system includes but is not limited to: thionyl chloride/methanol, thionyl chloride/ethanol and the like.

In another preferred embodiment, the chlorination reaction is carried out in the presence of a chlorination reagent.

In another preferred embodiment, the chlorination reagent includes but is not limited to: thionyl chloride, phosphorus pentachloride and N-chlorosuccinimide (NCS), etc.

In another preferred embodiment, the Grignard reaction is carried out in the presence of a Grignard reagent.

In another preferred embodiment, the Grignard reagent includes but is not limited to: methyl magnesium bromide, methyl magnesium chloride, methyl magnesium iodide and the like.

In another preferred embodiment, the bromination reaction is carried out in the presence of a bromination reagent.

In another preferred embodiment, the bromination reagent includes but is not limited to: elemental bromine, N-bromosuccinimide (NBS) and the like.

In the third aspect of the present invention, it provides a pharmaceutical composition comprising:
(A1) a first active ingredient, the first active ingredient comprises a therapeutically effective amount of one or more of the bis-benzyl isoquinoline compound of formula I of the first aspect, an enantiomer, a diastereomer, a racemate, and a pharmaceutically acceptable salt, a crystalline hydrate and a solvate thereof, and
(B) a pharmaceutically acceptable carrier.

In another preferred embodiment, the pharmaceutical composition further comprises (A2) a second active ingredient.

In another preferred embodiment, the second active ingredient is selected from the group consisting of: (Y1) an RNA replicase inhibitor (such as Remdesivir or GS-5734); (Y2) Lopinavir; (Y3) Ritonavir; and (Y4) Favipiravir; (Y5) Chloroquine, hydroxychloroquine, or a pharmaceutically acceptable salt thereof (such as chloroquine phosphate), (Y6) an antibody, (Y7) any combination of Y1 to Y6 described above.

In another preferred embodiment, the antibody includes anti-coronavirus antibody.

In the fourth aspect of the present invention, it provides a use of the bis-benzyl isoquinoline compound of the first aspect or the pharmaceutical composition of the third aspect, that is, for preparing an inhibitor for inhibiting virus replication; and/or a medicament for preventing and/or treating a related disease caused by a virus infection.

In another preferred embodiment, the virus is selected from: filamentous virus, flaviviridae virus, paramyxoviridae virus, arenaviridae virus, coronaviridae virus, or its combination.

In another preferred embodiment, the filamentous virus is selected from: Marburg virus, Ebola virus.

In another preferred embodiment, for the use, the coronavirus is selected from the group consisting of a coronavirus infecting a human, a severe acute respiratory syndrome coronavirus (SARS-CoV), a 2019 novel coronavirus (2019-nCoV or SARS-CoV-2), a Middle East respiratory syndrome coronavirus (MERS-CoV), a coronavirus causing common cold, and a combination thereof.

In another preferred embodiment, the coronavirus causing common cold is selected from: Human coronavirus OC₄₃, Human coronavirus 229E, Human coronavirus NL63, Human coronavirus HKUI.

In another preferred embodiment, the related disease caused by the coronavirus is selected from the group consisting of common cold caused by human coronavirus, infection with high-risk symptoms, respiratory tract infection, pneumonia and its complications, novel coronavirus pneumonia caused by SARS-CoV-2 (Corona Virus Disease 2019, COVID-19) and a combination thereof.

In another preferred embodiment, the related disease caused by 2019 novel coronavirus infection is selected from the group consisting of respiratory tract infection, pneumonia and complications thereof, or a combination thereof.

In another preferred embodiment, the use is for preparing (a) an inhibitor for inhibiting the replication of the 2019 novel coronavirus (SARS-CoV-2); and/or (b) a medicament for treating and/or preventing or alleviating a related disease caused by the infection of the 2019 novel coronavirus (SARS-CoV-2).

It should be understood that in the present invention, any of the technical features specifically described above and below (such as in the Examples) can be combined with each other, so as to constitute new or preferred technical solutions which will not redundantly be described one by one herein.

### DESCRIPTION OF FIGURES

No

### DETAILED DESCRIPTION OF THE INVENTION

After extensive and in-depth research and a large number of screens, the present inventors have for the first time unexpectedly developed a class of active ingredients that can effectively inhibit the replication of coronaviruses such as the 2019 novel coronavirus (SARS-CoV-2). Experiments show that the active ingredient (the bis-benzyl isoquinoline compound represented by formula I or a pharmaceutically acceptable salt thereof, or an enantiomer, a diastereomer, a racemate, or a crystaline hydrate, a solvate, or a mixture thereof) of the present invention can efficiently inhibit the replication and activity of a coronavirus such as a 2019 novel coronavirus (SARS-CoV-2). On above basis, the present invention has been completed.

Unexpectedly, compared with natural compounds such as berbamine, the bis-benzyl isoquinoline compound of formula I has significantly improved anti-coronavirus activity (the antiviral activity can be increased by about 10 times or more), therefore, it has a better therapeutic index SI and can be used for the prevention and treatment of coronaviruses such as SARS-CoV-2 viruses, and has a good clinical application prospect.

### TERMS

As used herein, “compounds of the present invention”, “bis-benzyl isoquinoline compounds”, “compounds of the present invention that inhibit coronavirus activity”, and “compounds of the present invention that inhibit coronavirus replication” are used interchangeably, and refer to bis-benzyl isoquinoline compounds that have excellent inhibition of coronavirus replication, including compounds shown in formula I, or a pharmaceutically acceptable salt thereof, or an enantiomer, diastereomer, racemate thereof, or a crystalline hydrate, solvate thereof, or a mixture thereof. The compounds of the present invention do not include natural compounds (e. g., berbamine, tetrandrine, Fangchinoline).

As used herein, “formulation of the present invention” refers to a formulation containing a compound of the present invention.

As used herein, the term “comprise” or variations thereof such as “comprising” or “comprises” and the like are understood to include stated elements or components, but not to exclude other elements or other components.

As used herein, the terms “novel coronavirus”, “2019-nCov” or “SARS-CoV-2” are used interchangeably, and the 2019 novel coronavirus is the 7th coronavirus known to infect humans and cause COVID-19, one of the serious infectious diseases threatening human health worldwide.

Halogen as used herein generally refers to fluorine, chlorine, bromine and iodine; preferably fluorine, chlorine or bromine; more preferably fluorine or chlorine.

C₁˜C₆ alkyl means a straight or branched chain saturated hydrocarbon group containing 1 to 6 carbon atoms, for example, methyl, ethyl, n-propyl, isopropyl, n-butyl, isobutyl, tert-butyl, sec-butyl, n-pentyl, 1-ethylpropyl, isopentyl, neopentyl, isohexyl, 3-methylpentyl or n-hexyl, etc., preferably methyl, ethyl, n-propyl, isopropyl, butyl, isobutyl or tert-butyl. In addition, C₁˜C₆ alkyl also includes C₃˜C₆ cycloalkyl, such as cyclopropyl, cyclobutyl, cycloalkyl, and cyclohexyl.

Halogenated C₁˜C₆ alkyl means a straight or branched chain saturated hydrocarbon group having 1 to 6 carbon atoms whose hydrogen atoms are substituted by one or more of the same or different halogen atoms, for example, trifluoromethyl, fluoromethyl, difluoromethyl, chloromethyl, bromomethyl, dichlorofluoromethyl, chloroethyl, bromopropyl, 2-chlorobutyl or pentafluoroethyl.

C₁˜C₆ alkoxy refers to a straight or branched chain alkoxy group containing 1 to 6 carbon atoms, for example, methoxy, ethoxy, n-propoxy, isopropoxy, n-butoxy, isobutoxy, tert-butoxy, sec-butoxy, n-pentyloxy, isopentyloxy, neopentyloxy, isohexyloxy, 3-methylpentyloxy or n-hexyloxy, preferably methoxy, ethoxy, n-propoxy, isopropoxy, n-butoxy, isobutoxy or tert-butoxy.

Halogenated C₁˜C₆ alkoxy means a straight or branched chain alkoxy having 1 to 6 carbon atoms whose hydrogen atoms are substituted by one or more of the same or different halogen atoms, for example, —OCF₃, —OCH₂CH₂Cl, —OCHBrCH₂Cl or —OCF₂CF₃.

C₁˜C₆ alkylthio refers to a straight or branched chain alkylthio group containing 1 to 6 carbon atoms, for example, methylthio, ethylthio, n-propylthio, isopropylthio, n-butylthio, isobutylthio, tert-butylthio, sec-butylthio, n-pentylthio, isopentylthio, neopentylthio or n-hexylthio, preferably methylthio, ethylthio, n-propylthio, isopropylthio, n-butylthio, isobutylthio or tert-butylthio.

C₂˜C₆ alkenyl refers to a straight or branched chain unsaturated hydrocarbon group containing 1-3 double bonds and 2-6 carbon atoms, including both cis and trans configurations, for example, vinyl, 1-propenyl, 2-propenyl, 1-methyl-1-propenyl, 2-methyl-1-propenyl, 2-methyl-2-propenyl, 1-butenyl, 2-butenyl, 3-butenyl, 1-pentenyl, 2-pentenyl, 3-pentenyl, 4-pentenyl, 1,3-butadienyl, 1,3-pentadienyl, 1-hexenyl, 2-hexenyl, 3-hexenyl, 4-hexenyl, 5-hexenyl, 3,3-dimethyl-1-propenyl, 2-ethyl-1-propenyl, or the like.

C₂-C₆ alkynyl refers to a straight or branched chain alkynyl containing 2-6 carbon atoms, for example, ethynyl, 2-propynyl, 2-butynyl, 3-butynyl, 1-methyl-2-propynyl, 2-pentynyl, 2-pentynyl or 2-hexynyl, etc.

C₂˜C₆ alkenyloxy refers to a straight or branched chain alkenyloxy containing 1-3 double bonds and 2-6 carbon atoms, such as vinyloxy, 1-propenyloxy, 1-methyl-1-propenyloxy, 2-methyl-1-propenyloxy, 1-pentenyloxy, 1,3-pentadienyloxy or 2-pentenyloxy, etc.

C₂˜C₆ alkynyloxy refers to a straight or branched chain alkynyloxy containing 2 to 6 carbon atoms, for example, ethynyloxy, 2-propynyloxy, 2-butynyloxy, 3-butynyloxy, 1-methyl-2-propynyloxy, 2-pentynyloxy or 2-hexynyloxy, etc.

C₁˜C₆ alkanoyl refers to a straight or branched chain alkanoyl having 1 to 6 carbon atoms, such as formyl, acetyl, propionyl, butyryl, isobutyryl, valeryl, t-butyryl or hexanoyl, etc.

Halogenated C₁˜C₆ alkanoyl refers to a straight or branched chain alkanoyl having 1 to 6 carbon atoms in which the hydrogen atom is substituted by one or more of the same or different halogen atoms, for example, trifluoroacetyl and the like.

Carbamoyl substituted by C₁˜C₆ alkyl means that the hydrogen atom on the carbamoyl is substituted by one or two of the same or different C₁˜C₆ alkyl (including C₃˜C₆ cycloalkyl), for example, —CONHMe, —CONHEt, —CON(Me)Et, —CONEt₂, —CONMe₂, or —CONH(C₃˜C₆ cycloalkyl).

Carbamoyl substituted by hydroxy C₁˜C₆ alkoxy C₁˜C₆ alkyl refers to that the hydrogen atom on the carbamoyl is substituted by one or two of the same or different hydroxy C₁˜C₆ alkoxy C₁˜C₆ alkyl, such as —CONHCH₂OCH₂OH, —CONHCH₂CH₂OCH₂CH₂OH, etc.

C₁˜C₆ alkoxycarbonyl substituted by a hydroxy C₁˜C₆ alkoxy refers to that one alkyl carbon atom of a C₁˜C₆ alkoxycarbonyl is bonded to the oxygen atom of a hydroxy C₁˜C₆ alkoxy, for example —COOCH₂OCH₂OH, —COOCH₂CH₂OCH₂CH₂OH, etc.

Hydroxy C₁˜C₆ alkyl refers to a linear or branched alkyl containing 1-6 carbon atoms in which a carbon atom is connected to a hydroxy, such as —CH₂OH, —CH₂CH₂OH, —CH(OH)CH₃, —CH₂CH₂CH₂OH, —CH₂CH₂CH₂CH₂OH or —CH₂CH(CH₃)CH₂OH, etc.

Amino C₁-C₆ alkyl refers to that a straight or branched alkyl containing 1 to 6 carbon atoms in which one carbon atom is bonded to an amino, such as —CH₂NH₂, —CH₂CH₂NH₂, —CH(NH₂)CH₃, —CH₂CH₂CH₂NH₂ or —CH₂CH₂CH₂CH₂NH₂, etc.

Amino C₁˜C₆ alkyl substituted by C₁˜C₆ alkyl refers to that the hydrogen atom on the amino is substituted by one or two of the same or different C₁˜C₆ alkyl, such as —CH₂NHMe or —CH₂CH₂NEt₂, etc.

Carbamoyl C₁˜C₆ alkyl refers to a straight or branched chain alkyl having 1 to 6 carbon atoms in which one carbon atom is attached to the carbonyl carbon of the carbamoyl, for example, —CH₂CONH₂, —CH₂CH₂CONH₂, —CH(CONH₂)CH₃ or —CH₂CH₂CH₂CONH₂ and the like.

Carbamoyl C₁-C₆ alkyl substituted by C₁-C₆ alkyl refers to that the amino hydrogen atom on carbamoyl C₁-C₆ alkyl is substituted by one or two same or different C₁-C₆ alkyl, such as —CH₂CONHMe, —CH₂CH₂CONHEt, —CH₂CH₂CONMe₂ or —CH₂CONEt₂, etc.

Cyano C₁˜C₆ alkyl refers to a straight or branched chain alkyl having 1 to 6 carbon atoms in which one carbon atom is attached to a cyano, such as cyanomethyl, 2-cyanoethyl, 1-cyanoethyl, 3-cyanopropyl, 4-cyanobutyl or 5-cyanopentyl, etc.

Carboxyl C₁˜C₆ alkyl refers to a straight or branched chain alkyl containing 1 to 6 carbon atoms in which one carbon atom is bonded to a carboxyl, such as carboxymethyl, 2-carboxyethyl, 1-carboxyethyl, 3-carboxypropyl, 4-carboxybutyl or 5-carboxypentyl and the like, for example

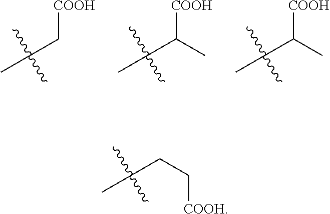

C₁˜C₆ alkylsulfonyl refers to a straight or branched chain alkylsulfonyl having 1 to 6 carbon atoms, such as methanesulfonyl, ethanesulfonyl or propanesulfonyl and the like.

Halogenated C₁˜C₆ alkanesulfonyl refers to a straight or branched chain alkanesulfonyl having 1 to 6 carbon atoms in which the hydrogen atom is substituted by one or more of the same or different halogen atoms, for example, trifluoromethanesulfonyl and the like.

Amino substituted by C₁˜C₆ alkyl refers to that the hydrogen atom on the amino is substituted by one or two same or different C₁˜C₆ alkyl or C₁˜C₆ alkanoyl, such as —NHMe or —NEt₂.

C₃-C₆ cycloalkyl refers to a saturated cyclic hydrocarbon containing 3 to 6 carbon atoms, such as cyclopropyl, cyclobutyl, cyclopentyl, cyclohexyl and the like.

C₃-C₆ cycloalkoxy refers to a saturated cyclic hydrocarbyloxy containing 3 to 6 carbon atoms, such as cyclopropoxy, cyclobutoxy, cyclopentyloxy, cyclohexyloxy and the like. C₂-C₁₀ ester group refers to a saturated or unsaturated straight or branched chain ester group containing 2-10 carbon atoms, such as —COO—(C₁-C₉ alkyl), —COO—(C₂-C₅ alkenyl) or —COO—(C₃-C₉ cycloalkyl), or —O—CO—(C₁-C₉ alkyl), —O—CO—(C₂-C₈ alkenyl) or —O—CO—(C₃-C₉ cycloalkyl), etc., and the alkyl, alkenyl or cycloalkyl may be substituted or unsubstituted (e. g., halogenated).

### Bis-Benzyl Isoquinoline Compounds

Bis-benzyl isoquinoline alkaloids are a very important class of natural products, and many monomers with clear structure have been used as drugs in clinic, such as berbamine, tetrandrine, and so on.

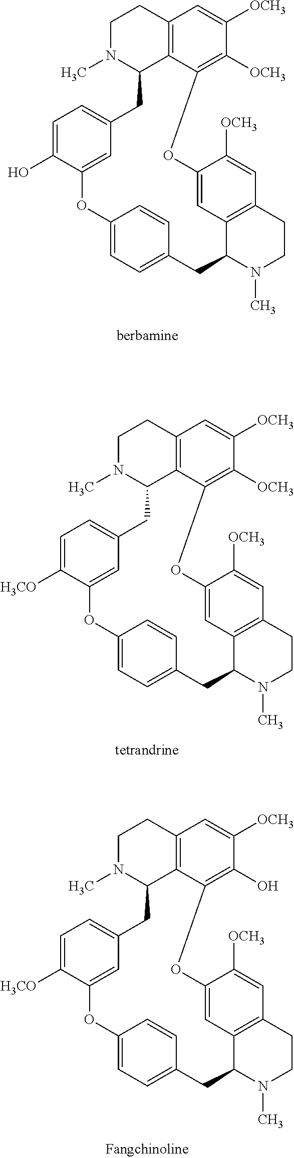 [SMILES: COc1cc2c3cc1Oc1c(OC)c(OC)cc4c1[C@@H](Cc1ccc(O)c(c1)Oc1ccc(cc1)C[C@@H]3N(C)CC2)N(C)CC4 ; COc1ccc2cc1Oc1ccc(cc1)C[C@H]1c3cc(c(OC)cc3CCN1C)Oc1c(OC)c(OC)cc3c1[C@H](C2)N(C)CC3 ; COc1ccc2cc1Oc1ccc(cc1)C[C@H]1c3cc(c(OC)cc3CCN1C)Oc1c(O)c(OC)cc3c1[C@@H](C2)N(C)CC3]

Berbamine is a bisbenzyl isoquinoline alkaloid extracted from the root of Berberis amurensis Rupr., a Chinese herbal medicine for clearing heat and drying dampness, purging fire for removing toxin. Berbamine has a variety of pharmacological effects, such as anti-inflammatory, immune regulation, increase myocardial contractility, prevent myocardial ischemia-reperfusion injury, anti-arrhythmia, antihypertensive, anti-tumor, prevention/treatment of cataract, its hydrochloride as a leukocyte proliferation drug has been used in clinical for a long time, oral medication 3 times a day, 4 tablets each time, 28 mg each tablet.

Tetrandrine, also known as tetrandrine, is a bisbenzyl isoquinoline alkaloid extracted from the root of the Chinese herbal medicine Fangji. Tetrandrine is an anti-hypertensive drug, which is mainly used for the treatment of mild to moderate hypertension, and can also be used for hypertensive crisis.

### Compounds of the Invention

The compound of the present invention is a bisbenzyl isoquinoline compound of formula (I) or a pharmaceutically acceptable salt thereof, or an enantiomer, a diastereomer, a racemate, a crystalline hydrate, a solvate, or a mixture thereof, wherein the bisbenzyl isoquinoline compound is a compound of formula (I):

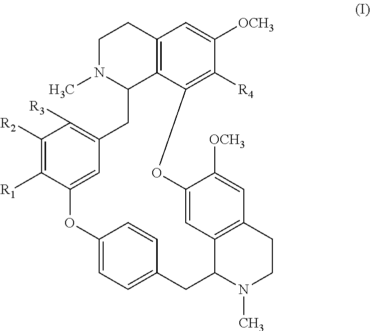

wherein R₁, R₂, R₃ and R₄ are as defined above.

Preferably, the compounds of formula I of the present invention are non-natural compounds.

Preferably, the compounds of the invention do not contain phenolic hydroxy groups (e. g., R₁, R₂ and R₃ are not phenolic hydroxy groups, and/or R₄ are not phenolic hydroxy groups), or R₁ and/or R₂ are not phenolic hydroxy groups.

In the present invention, the inventors unexpectedly found that when the compound does not contain a phenolic hydroxy group, its inhibitory activity against coronavirus SARS-CoV-2 is actually significantly improved, and its EC₅₀ value is about 10 times lower than the EC₅₀ value of berbamine hydrochloride.

Preferably, the compound of the invention is selected from Table A:

TABLE A

| | |
|---|---|
| 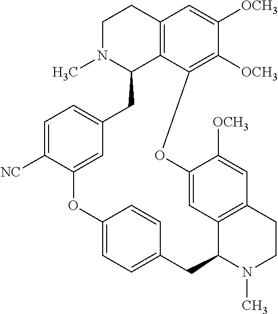 [SMILES: COc1cc2c3cc1Oc1c(OC)c(OC)cc4c1[C@@H](Cc1ccc(C#N)c(c1)Oc1ccc(cc1)C[C@@H]3N(C)CC2)N(C)CC4] | 1 |
| 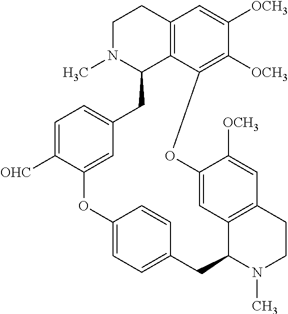 [SMILES: COc1cc2c3cc1Oc1c(OC)c(OC)cc4c1[C@@H](Cc1ccc(C=O)c(c1)Oc1ccc(cc1)C[C@@H]3N(C)CC2)N(C)CC4] | 2 |
| 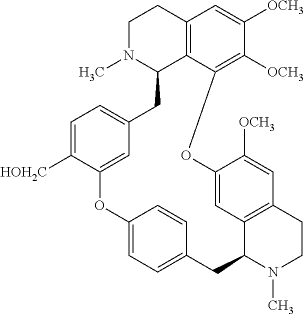 [SMILES: COc1cc2c3cc1Oc1c(OC)c(OC)cc4c1[C@@H](Cc1ccc(CO)c(c1)Oc1ccc(cc1)C[C@@H]3N(C)CC2)N(C)CC4] | 3 |
| 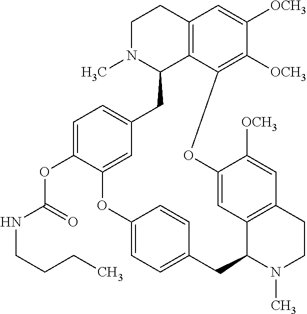 [SMILES: CCCCNC(=O)Oc1ccc2cc1Oc1ccc(cc1)C[C@H]1c3cc(c(OC)cc3CCN1C)Oc1c(OC)c(OC)cc3c1[C@@H](C2)N(C)CC3] | 4 |
| 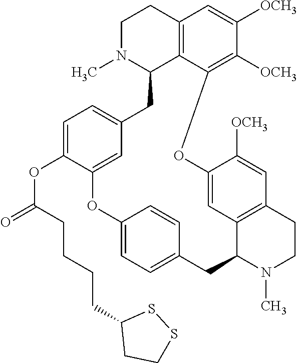 [SMILES: COc1cc2c3cc1Oc1c(OC)c(OC)cc4c1[C@@H](Cc1ccc(OC(=O)CCCC[C@H]5CCSS5)c(c1)Oc1ccc(cc1)C[C@@H]3N(C)CC2)N(C)CC4] | 5 |
| 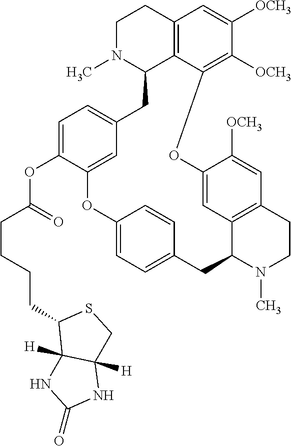 [SMILES: COc1cc2c3cc1Oc1c(OC)c(OC)cc4c1[C@@H](Cc1ccc(OC(=O)CCCC[C@@H]5SC[C@@H]6NC(=O)N[C@@H]65)c(c1)Oc1ccc(cc1)C[C@@H]3N(C)CC2)N(C)CC4] | 6 |
| 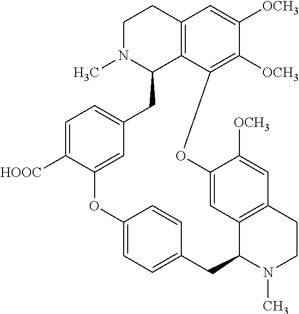 [SMILES: COc1cc2c3cc1Oc1c(OC)c(OC)cc4c1[C@@H](Cc1ccc(C(=O)O)c(c1)Oc1ccc(cc1)C[C@@H]3N(C)CC2)N(C)CC4] | 7 |
| 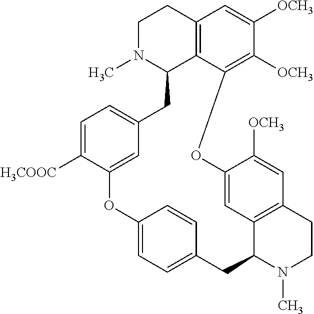 | 8 |
| 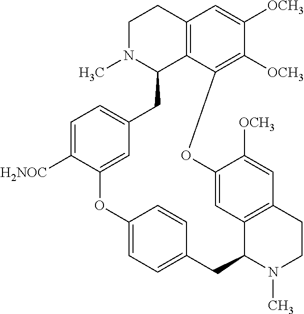 [SMILES: COc1cc2c3cc1Oc1c(OC)c(OC)cc4c1[C@@H](Cc1ccc(C(N)=O)c(c1)Oc1ccc(cc1)C[C@@H]3N(C)CC2)N(C)CC4] | 9 |
| 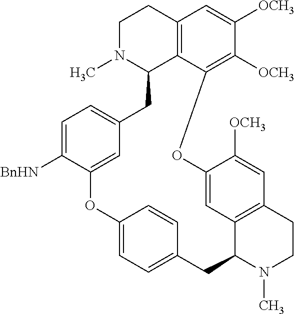 | 10 |
| 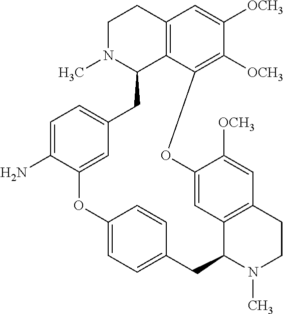 [SMILES: COc1cc2c3cc1Oc1c(OC)c(OC)cc4c1[C@@H](Cc1ccc(N)c(c1)Oc1ccc(cc1)C[C@@H]3N(C)CC2)N(C)CC4] | 11 |
| 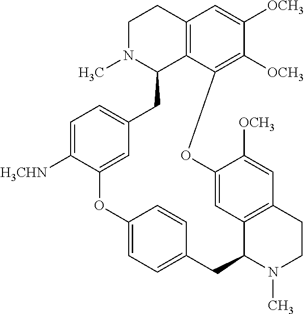 | 12 |
| 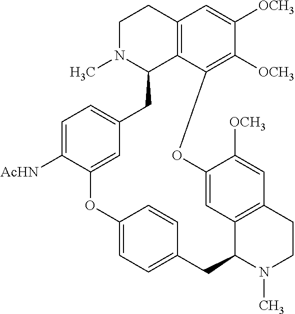 [SMILES: COc1cc2c3cc1Oc1c(OC)c(OC)cc4c1[C@@H](Cc1ccc(NC(C)=O)c(c1)Oc1ccc(cc1)C[C@@H]3N(C)CC2)N(C)CC4] | 13 |
| 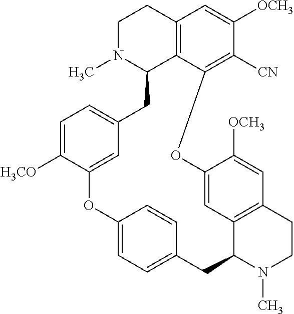 [SMILES: COc1ccc2cc1Oc1ccc(cc1)C[C@H]1c3cc(c(OC)cc3CCN1C)Oc1c(C#N)c(OC)cc3c1[C@@H](C2)N(C)CC3] | 14 |
| 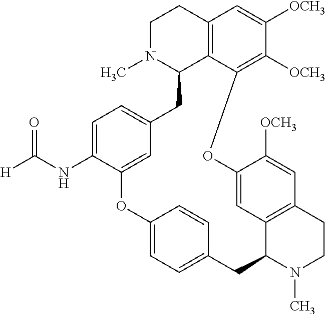 [SMILES: COc1cc2c3cc1Oc1c(OC)c(OC)cc4c1[C@@H](Cc1ccc(NC=O)c(c1)Oc1ccc(cc1)C[C@@H]3N(C)CC2)N(C)CC4] | 15 |
| 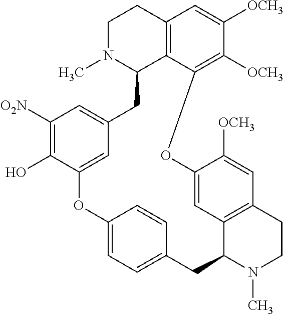 [SMILES: COc1cc2c3cc1Oc1c(OC)c(OC)cc4c1[C@@H](Cc1cc(c(O)c([N+](=O)[O-])c1)Oc1ccc(cc1)C[C@@H]3N(C)CC2)N(C)CC4] | 16 |
| 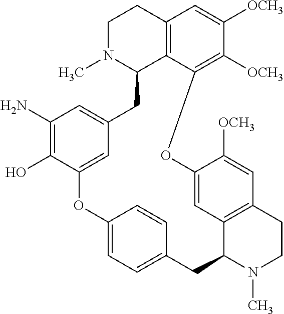 [SMILES: COc1cc2c3cc1Oc1c(OC)c(OC)cc4c1[C@@H](Cc1cc(N)c(O)c(c1)Oc1ccc(cc1)C[C@@H]3N(C)CC2)N(C)CC4] | 17 |
| 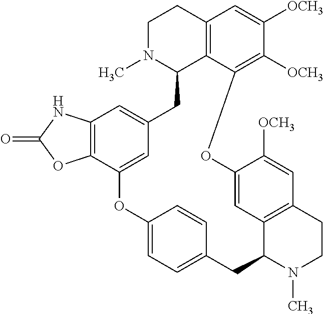 [SMILES: COc1cc2c3cc1Oc1c(OC)c(OC)cc4c1[C@@H](Cc1cc(c5oc(=O)[nH]c5c1)Oc1ccc(cc1)C[C@@H]3N(C)CC2)N(C)CC4] | 18 |
| 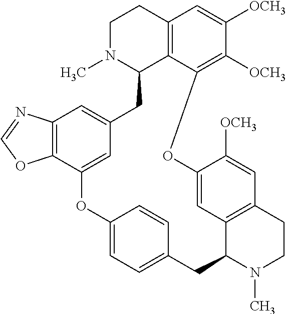 [SMILES: COc1cc2c3cc1Oc1c(OC)c(OC)cc4c1[C@@H](Cc1cc(c5ocnc5c1)Oc1ccc(cc1)C[C@@H]3N(C)CC2)N(C)CC4] | 19 |
| 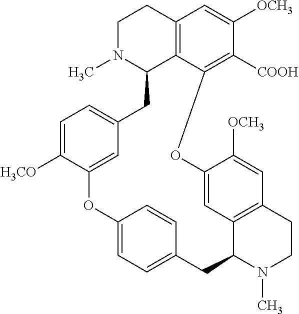 [SMILES: COc1ccc2cc1Oc1ccc(cc1)C[C@H]1c3cc(c(OC)cc3CCN1C)Oc1c(C(=O)O)c(OC)cc3c1[C@@H](C2)N(C)CC3] | 20 |
| 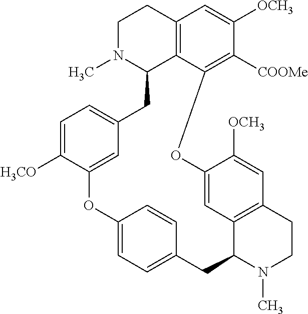 [SMILES: COC(=O)c1c(OC)cc2c3c1Oc1cc4c(cc1OC)CCN(C)[C@H]4Cc1ccc(cc1)Oc1cc(ccc1OC)C[C@H]3N(C)CC2] | 21 |
| 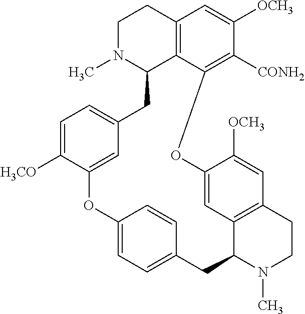 [SMILES: COc1ccc2cc1Oc1ccc(cc1)C[C@H]1c3cc(c(OC)cc3CCN1C)Oc1c(C(N)=O)c(OC)cc3c1[C@@H](C2)N(C)CC3] | 23 |
| 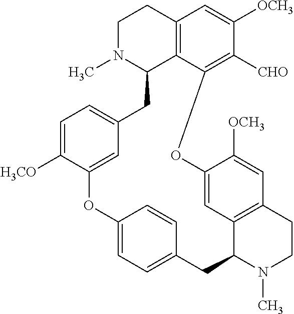 [SMILES: COc1ccc2cc1Oc1ccc(cc1)C[C@H]1c3cc(c(OC)cc3CCN1C)Oc1c(C=O)c(OC)cc3c1[C@@H](C2)N(C)CC3] | 23 |
| 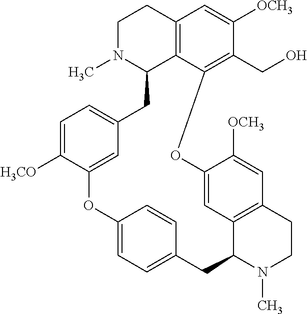 [SMILES: COc1ccc2cc1Oc1ccc(cc1)C[C@H]1c3cc(c(OC)cc3CCN1C)Oc1c(CO)c(OC)cc3c1[C@@H](C2)N(C)CC3] | 24 |
| 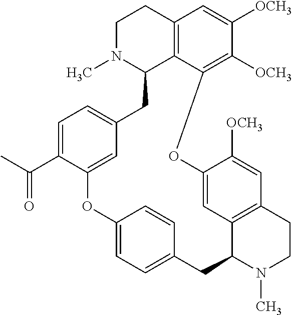 [SMILES: COc1cc2c3cc1Oc1c(OC)c(OC)cc4c1[C@@H](Cc1ccc(C(C)=O)c(c1)Oc1ccc(cc1)C[C@@H]3N(C)CC2)N(C)CC4] | 25 |
| 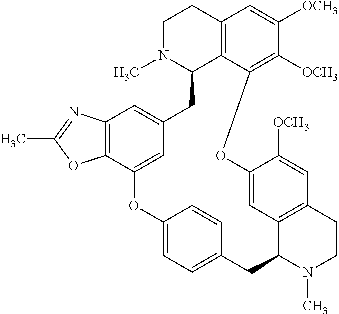 [SMILES: COc1cc2c3cc1Oc1c(OC)c(OC)cc4c1[C@@H](Cc1cc(c5oc(C)nc5c1)Oc1ccc(cc1)C[C@@H]3N(C)CC2)N(C)CC4] | 26 |
| 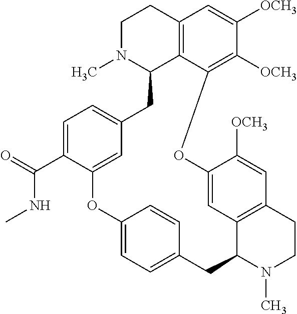 [SMILES: CNC(=O)c1ccc2cc1Oc1ccc(cc1)C[C@H]1c3cc(c(OC)cc3CCN1C)Oc1c(OC)c(OC)cc3c1[C@@H](C2)N(C)CC3] | 27 |
| 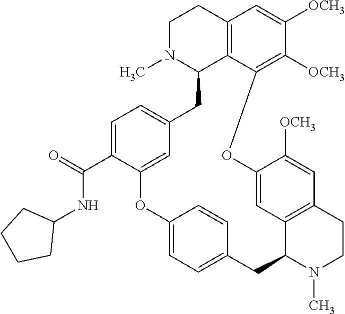 [SMILES: COc1cc2c3cc1Oc1c(OC)c(OC)cc4c1[C@@H](Cc1ccc(C(=O)NC5CCCC5)c(c1)Oc1ccc(cc1)C[C@@H]3N(C)CC2)N(C)CC4] | 28 |
| 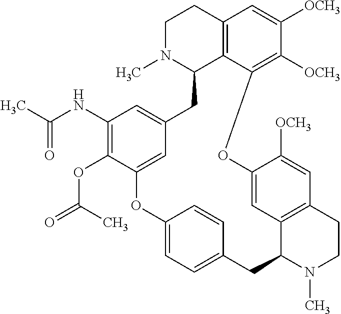 [SMILES: COc1cc2c3cc1Oc1c(OC)c(OC)cc4c1[C@@H](Cc1cc(NC(C)=O)c(OC(C)=O)c(c1)Oc1ccc(cc1)C[C@@H]3N(C)CC2)N(C)CC4] | 29 |
| 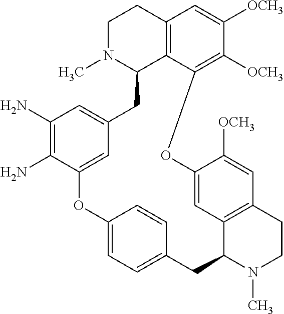 [SMILES: COc1cc2c3cc1Oc1c(OC)c(OC)cc4c1[C@@H](Cc1cc(N)c(N)c(c1)Oc1ccc(cc1)C[C@@H]3N(C)CC2)N(C)CC4] | 30 |
| 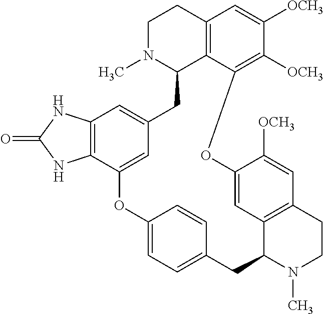 [SMILES: COc1cc2c3cc1Oc1c(OC)c(OC)cc4c1[C@@H](Cc1cc(c5[nH]c(=O)[nH]c5c1)Oc1ccc(cc1)C[C@@H]3N(C)CC2)N(C)CC4] | 31 |
| 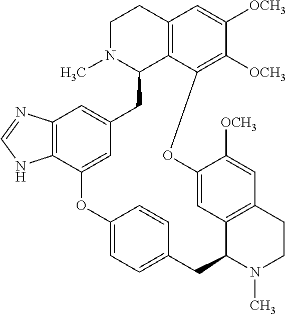 [SMILES: COc1cc2c3cc1Oc1c(OC)c(OC)cc4c1[C@@H](Cc1cc(c5[nH]cnc5c1)Oc1ccc(cc1)C[C@@H]3N(C)CC2)N(C)CC4] | 32 |
| 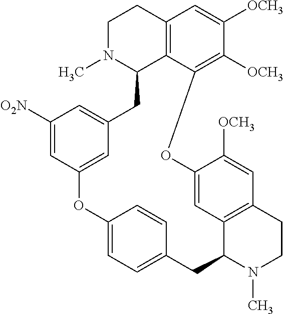 [SMILES: COc1cc2c3cc1Oc1c(OC)c(OC)cc4c1[C@@H](Cc1cc(cc([N+](=O)[O-])c1)Oc1ccc(cc1)C[C@@H]3N(C)CC2)N(C)CC4] | 33 |
| 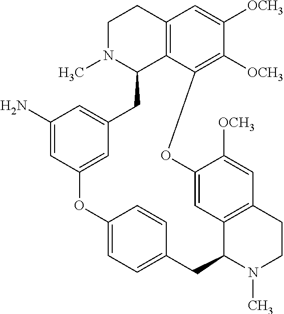 [SMILES: COc1cc2c3cc1Oc1c(OC)c(OC)cc4c1[C@@H](Cc1cc(N)cc(c1)Oc1ccc(cc1)C[C@@H]3N(C)CC2)N(C)CC4] | 34 |
| 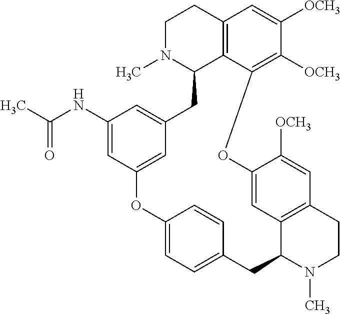 [SMILES: COc1cc2c3cc1Oc1c(OC)c(OC)cc4c1[C@@H](Cc1cc(NC(C)=O)cc(c1)Oc1ccc(cc1)C[C@@H]3N(C)CC2)N(C)CC4] | 35 |
| 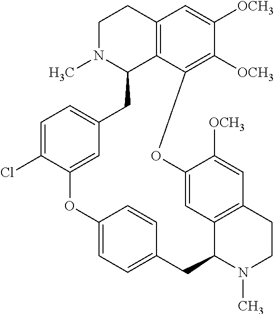 [SMILES: COc1cc2c3cc1Oc1c(OC)c(OC)cc4c1[C@@H](Cc1ccc(Cl)c(c1)Oc1ccc(cc1)C[C@@H]3N(C)CC2)N(C)CC4] | 36 |
| 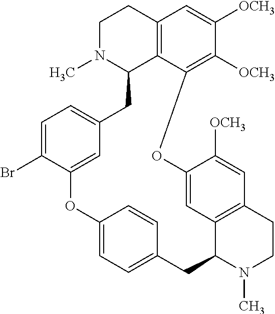 [SMILES: COc1cc2c3cc1Oc1c(OC)c(OC)cc4c1[C@@H](Cc1ccc(Br)c(c1)Oc1ccc(cc1)C[C@@H]3N(C)CC2)N(C)CC4] | 37 |
| 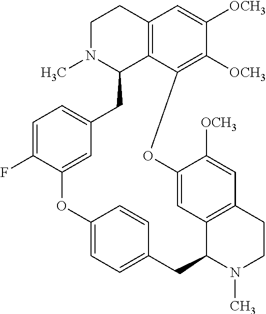 [SMILES: COc1cc2c3cc1Oc1c(OC)c(OC)cc4c1[C@@H](Cc1ccc(F)c(c1)Oc1ccc(cc1)C[C@@H]3N(C)CC2)N(C)CC4] | 38 |
| 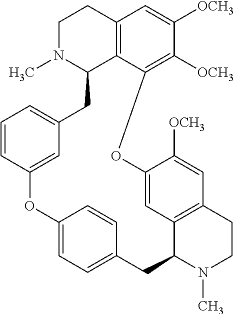 [SMILES: COc1cc2c3cc1Oc1c(OC)c(OC)cc4c1[C@@H](Cc1cccc(c1)Oc1ccc(cc1)C[C@@H]3N(C)CC2)N(C)CC4] | 39 |
| 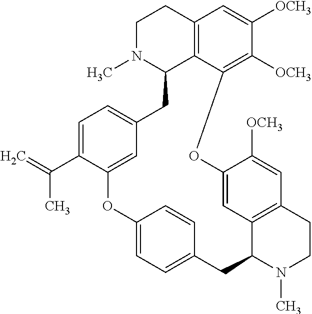 [SMILES: C=C(C)c1ccc2cc1Oc1ccc(cc1)C[C@H]1c3cc(c(OC)cc3CCN1C)Oc1c(OC)c(OC)cc3c1[C@@H](C2)N(C)CC3] | 40 |
| 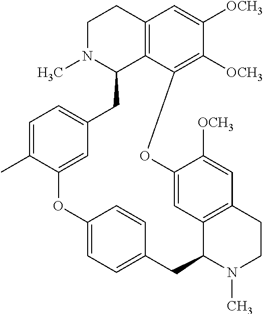 [SMILES: COc1cc2c3cc1Oc1c(OC)c(OC)cc4c1[C@@H](Cc1ccc(C)c(c1)Oc1ccc(cc1)C[C@@H]3N(C)CC2)N(C)CC4] | 41 |
| 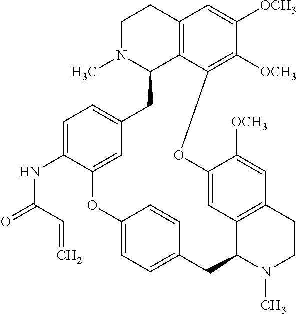 [SMILES: C=CC(=O)Nc1ccc2cc1Oc1ccc(cc1)C[C@H]1c3cc(c(OC)cc3CCN1C)Oc1c(OC)c(OC)cc3c1[C@@H](C2)N(C)CC3] | 42 |
| 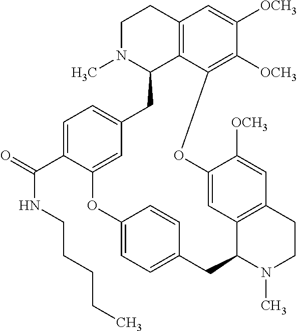 [SMILES: CCCCCNC(=O)c1ccc2cc1Oc1ccc(cc1)C[C@H]1c3cc(c(OC)cc3CCN1C)Oc1c(OC)c(OC)cc3c1[C@@H](C2)N(C)CC3] | 43 |
| 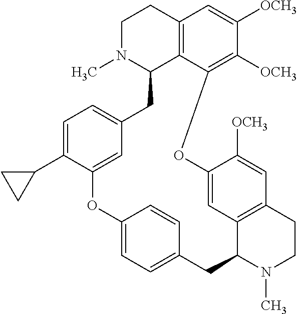 [SMILES: COc1cc2c3cc1Oc1c(OC)c(OC)cc4c1[C@@H](Cc1ccc(C5CC5)c(c1)Oc1ccc(cc1)C[C@@H]3N(C)CC2)N(C)CC4] | 44 |
| 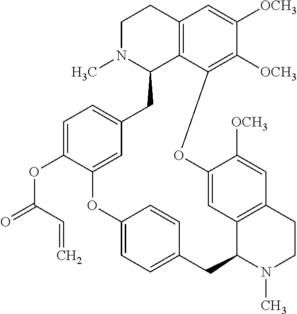 [SMILES: C=CC(=O)Oc1ccc2cc1Oc1ccc(cc1)C[C@H]1c3cc(c(OC)cc3CCN1C)Oc1c(OC)c(OC)cc3c1[C@@H](C2)N(C)CC3] | 45 |
| 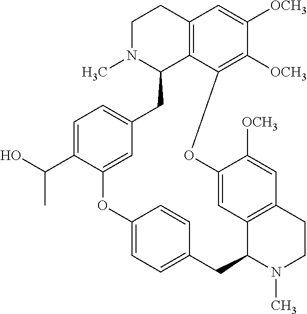 [SMILES: COc1cc2c3cc1Oc1c(OC)c(OC)cc4c1[C@@H](Cc1ccc(C(C)O)c(c1)Oc1ccc(cc1)C[C@@H]3N(C)CC2)N(C)CC4] | 46 |
| 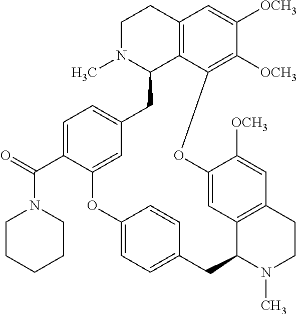 [SMILES: COc1cc2c3cc1Oc1c(OC)c(OC)cc4c1[C@@H](Cc1ccc(C(=O)N5CCCCC5)c(c1)Oc1ccc(cc1)C[C@@H]3N(C)CC2)N(C)CC4] | 47 |
| 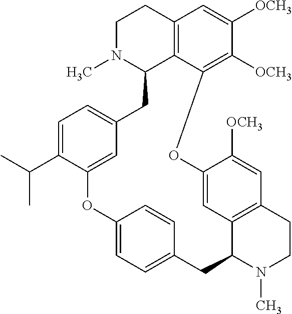 [SMILES: COc1cc2c3cc1Oc1c(OC)c(OC)cc4c1[C@@H](Cc1ccc(C(C)C)c(c1)Oc1ccc(cc1)C[C@@H]3N(C)CC2)N(C)CC4] | 48 |
| 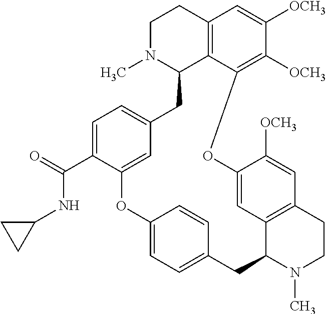 [SMILES: COc1cc2c3cc1Oc1c(OC)c(OC)cc4c1[C@@H](Cc1ccc(C(=O)NC5CC5)c(c1)Oc1ccc(cc1)C[C@@H]3N(C)CC2)N(C)CC4] | 49 |
| 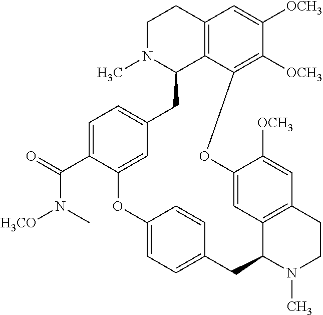 [SMILES: COc1cc2c3cc1Oc1c(OC)c(OC)cc4c1[C@@H](Cc1ccc(C(=O)N(C)OC)c(c1)Oc1ccc(cc1)C[C@@H]3N(C)CC2)N(C)CC4] | 50 |
| 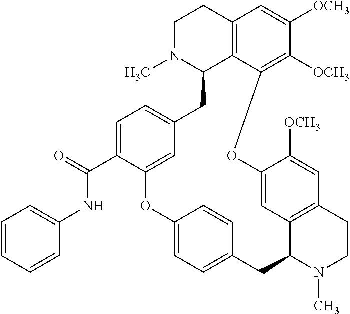 [SMILES: COc1cc2c3cc1Oc1c(OC)c(OC)cc4c1[C@@H](Cc1ccc(C(=O)Nc5ccccc5)c(c1)Oc1ccc(cc1)C[C@@H]3N(C)CC2)N(C)CC4] | 51 |
| 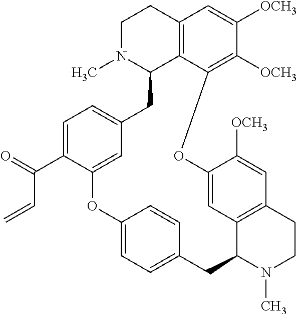 [SMILES: C=CC(=O)c1ccc2cc1Oc1ccc(cc1)C[C@H]1c3cc(c(OC)cc3CCN1C)Oc1c(OC)c(OC)cc3c1[C@@H](C2)N(C)CC3] | 52 |
| 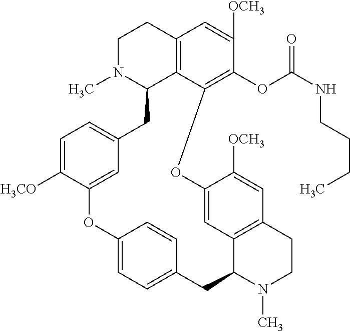 [SMILES: CCCCNC(=O)Oc1c(OC)cc2c3c1Oc1cc4c(cc1OC)CCN(C)[C@H]4Cc1ccc(cc1)Oc1cc(ccc1OC)C[C@H]3N(C)CC2] | 53 |
| 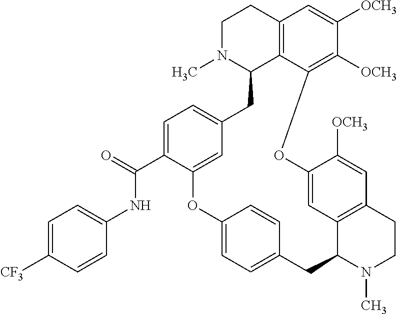 [SMILES: COc1cc2c3cc1Oc1c(OC)c(OC)cc4c1[C@@H](Cc1ccc(C(=O)Nc5ccc(C(F)(F)F)cc5)c(c1)Oc1ccc(cc1)C[C@@H]3N(C)CC2)N(C)CC4] | 54 |
| 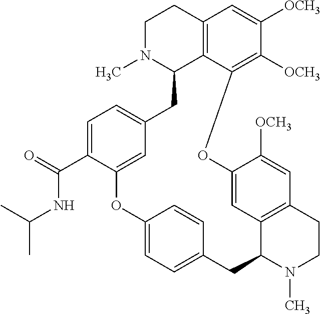 [SMILES: COc1cc2c3cc1Oc1c(OC)c(OC)cc4c1[C@@H](Cc1ccc(C(=O)NC(C)C)c(c1)Oc1ccc(cc1)C[C@@H]3N(C)CC2)N(C)CC4] | 55 |
| 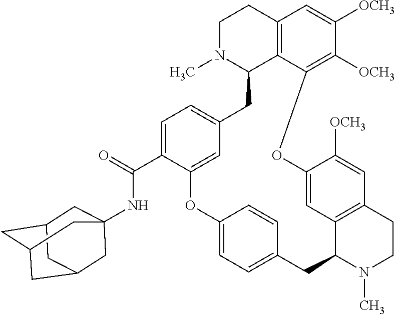 [SMILES: COc1cc2c3cc1Oc1c(OC)c(OC)cc4c1[C@@H](Cc1ccc(C(=O)NC56CC7CC(CC(C7)C5)C6)c(c1)Oc1ccc(cc1)C[C@@H]3N(C)CC2)N(C)CC4] | 56 |
| 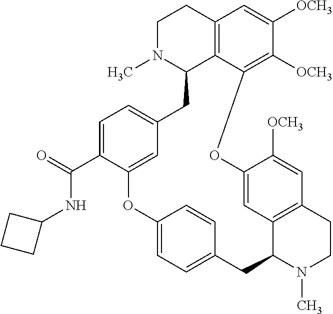 [SMILES: COc1cc2c3cc1Oc1c(OC)c(OC)cc4c1[C@@H](Cc1ccc(C(=O)NC5CCC5)c(c1)Oc1ccc(cc1)C[C@@H]3N(C)CC2)N(C)CC4] | 57 |
| 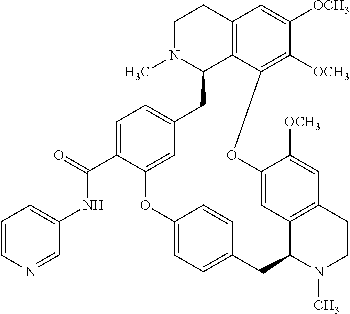 [SMILES: COc1cc2c3cc1Oc1c(OC)c(OC)cc4c1[C@@H](Cc1ccc(C(=O)Nc5cccnc5)c(c1)Oc1ccc(cc1)C[C@@H]3N(C)CC2)N(C)CC4] | 58 |
| 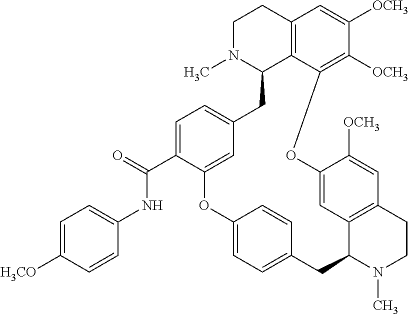 [SMILES: COc1ccc(NC(=O)c2ccc3cc2Oc2ccc(cc2)C[C@H]2c4cc(c(OC)cc4CCN2C)Oc2c(OC)c(OC)cc4c2[C@@H](C3)N(C)CC4)cc1] | 59 |
| 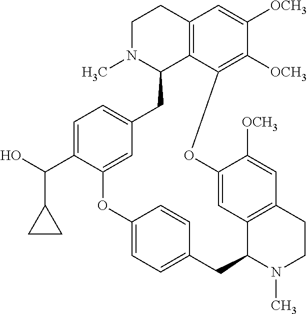 [SMILES: COc1cc2c3cc1Oc1c(OC)c(OC)cc4c1[C@@H](Cc1ccc(C(O)C5CC5)c(c1)Oc1ccc(cc1)C[C@@H]3N(C)CC2)N(C)CC4] | 60 |
| 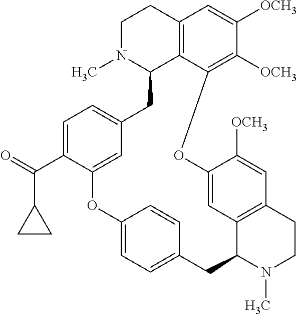 [SMILES: COc1cc2c3cc1Oc1c(OC)c(OC)cc4c1[C@@H](Cc1ccc(C(=O)C5CC5)c(c1)Oc1ccc(cc1)C[C@@H]3N(C)CC2)N(C)CC4] | 61 |
| 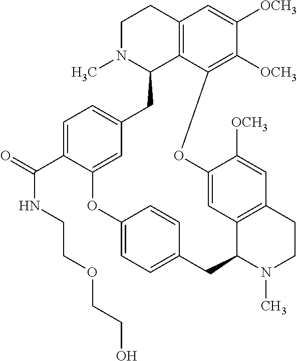 [SMILES: COc1cc2c3cc1Oc1c(OC)c(OC)cc4c1[C@@H](Cc1ccc(C(=O)NCCOCCO)c(c1)Oc1ccc(cc1)C[C@@H]3N(C)CC2)N(C)CC4] | 62 |
| 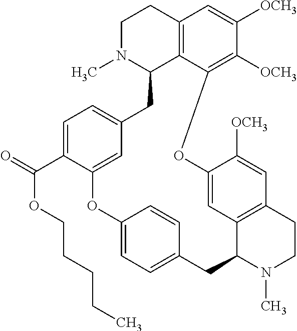 [SMILES: CCCCCOC(=O)c1ccc2cc1Oc1ccc(cc1)C[C@H]1c3cc(c(OC)cc3CCN1C)Oc1c(OC)c(OC)cc3c1[C@@H](C2)N(C)CC3] | 63 |
| 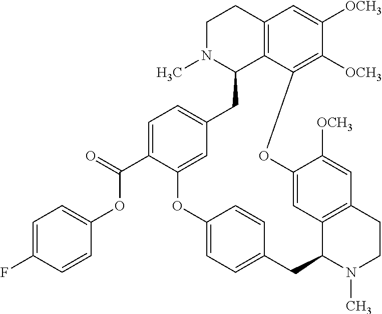 [SMILES: COc1cc2c3cc1Oc1c(OC)c(OC)cc4c1[C@@H](Cc1ccc(C(=O)Oc5ccc(F)cc5)c(c1)Oc1ccc(cc1)C[C@@H]3N(C)CC2)N(C)CC4] | 64 |
| 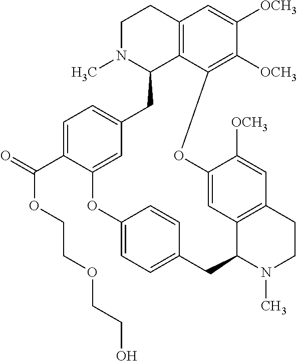 [SMILES: COc1cc2c3cc1Oc1c(OC)c(OC)cc4c1[C@@H](Cc1ccc(C(=O)OCCOCCO)c(c1)Oc1ccc(cc1)C[C@@H]3N(C)CC2)N(C)CC4] | 65 |

The “pharmaceutically acceptable salt” is a conventional non-toxic salt formed by reacting the active compound of the present invention with an inorganic acid or an organic acid. For example, conventional non-toxic salts can be prepared by reacting the active compound of the present invention with an inorganic or organic acid, the inorganic acid includes hydrochloric acid, hydrobromic acid, sulfuric acid, nitric acid, aminosulfonic acid, phosphoric acid, and the like, and the organic acid includes citric acid, tartaric acid, lactic acid, pyruvic acid, acetic acid, benzenesulfonic acid, p-toluenesulfonic acid, methanesulfonic acid, naphthalene sulfonic acid, ethanesulfonic acid, naphthalene disulfonic acid, maleic acid, malic acid, malonic acid, fumaric acid, succinic acid, propionic acid, oxalic acid, trifluoroacetic acid, stearic acid, pamoic acid, hydroxymaleic acid, phenylacetic acid, benzoic acid, salicylic acid, glutamic acid, ascorbic acid, p-aminobenzenesulfonic acid, 2-acetoxybenzoic acid and hydroxyethanesulfonic acid, etc.; alternatively, the sodium, zinc, potassium, calcium, aluminium or ammonium salts can be formed by reacting inorganic bases with the esters formed by reacting the active compounds of the invention with propionic acid, oxalic acid, malonic acid, succinic acid, fumaric acid, maleic acid, lactic acid, malic acid, tartaric acid, citric acid, aspartic acid or glutamic acid; or the corresponding inorganic acid salt formed with hydrochloric acid, hydrobromic acid, hydrofluoric acid, sulfuric acid, nitric acid or phosphoric acid or the corresponding organic acid salt formed with formic acid, acetic acid, picric acid, methanesulfonic acid or ethanesulfonic acid after the active compound of the present invention forms an ester with lysine, arginine and ornithine; or the sodium salt, zinc salt, potassium salt, calcium salt, aluminum salt or ammonium salt formed by the carboxyl/phenolic hydroxy group in the active compound molecule of the present invention and inorganic base.

In addition, the active ingredients of the present invention are also particularly suitable for use in combination with other drugs against coronaviruses. Representative other anti-coronavirus drugs include (but are not limited to): interferon, RNA-dependent RNA polymerase inhibitors (such as Remdesivir (GS-5734), favipiravir, Galidesivir, GS-441524, NHC, EIDD-2801); 3CL protease inhibitors (such as GC-376), Lopinavir, Ritonavir, Nelfinavir; Chloroquine, hydroxychloroquine, cyclosporine, Carrimycin, baicalin, baicalein, Naphthoquine, Ciclesonide, Ribavirin, Penciclovir, Leflunomide, Teriflunomide, nafamostat, nitazoxanide, Darunavir, Arbidol, Camostat, Niclosamide, Ivermectin, baricitinib, Ruxolitinib, Dasatinib, Saquinavir, Beclabuvir, Simeprevir, or a pharmaceutically acceptable salt thereof, or a combination thereof. The interferon includes one or more of interferon alpha-2a, interferon alpha-2b, interferon alpha-n1, interferon alpha-n3, interferon beta-1a, and interferon beta-1b.

In addition, since SARS-CoV-2 infection can cause acute lung injury, inflammatory reaction and even cytokine storm, the active ingredient of the present invention is also particularly suitable for use in combination with drugs having ameliorating acute lung injury, anti-inflammatory action or immunomodulatory action. Representative drugs include but are not limited to Zinc, Fingolimod, Vitamin C, Olmesartan Medoxomil, valsartan, Losartan, Thalidomide, glycyrrhizic acid, Artemisinin, dihydroartemisinin, Artesunate, Artemisone, Azithromycin, Escin, Naproxen.

Preferably, the active ingredient of the present invention is used in combination with an artemisinin-based drug (one or more of artemisinin, dihydroartemisinin, artesunate, artemisone). A large number of studies have shown that artemisinin drugs have multiple anti-inflammatory and immunomodulatory mechanisms, which can achieve anti-inflammatory and immunomodulatory functions by inhibiting T cell proliferation and activation, inhibiting B cell activation and antibody production, increasing regulatory T cells and reducing the release of proinflammatory cytokines. It is expected to alleviate the immune injury symptoms caused by novel coronavirus (SARS-CoV-2) infection.

Preferably, the active ingredients of the present invention are combined with artemisinin drugs (one or more of artemisinin, dihydroartemisinin, artesunate, artemisone) and azithromycin.

The active ingredient of the present invention can inhibit the infection activity of a novel coronavirus such as SARS-CoV-2. Therefore, when the active ingredient of the present invention is administered or given therapeutically, infection with the 2019 novel coronavirus (SARS-CoV-2) can be inhibited, thereby achieving an antiviral effect.

### Preparation Method

A method for preparing the bisbenzyl isoquinoline compound of the present invention is selected from one or more of the following methods:
a) the bis-benzyl isoquinoline compound is obtained by alkylation reaction between a phenolic hydroxy-containing bis-benzyl isoquinoline as a raw material and an alkylation reagent;
b) the bis-benzyl isoquinoline compound is obtained by an acylation reaction between a phenolic hydroxy-containing bis-benzyl isoquinoline as a raw material and an acylation reagent;

In another preferred embodiment, the method is that using berbamine as a raw material, undergoing a condensation acylation reaction with a carboxylic acid to obtain the bis-benzyl isoquinoline compound.

In another preferred embodiment, the condensation acylation reaction is carried out in the presence of a condensing agent, and the condensing agent includes but is not limited to: N, N′-dicyclohexylcarbodiimide (DCC), 1-ethyl-3-(3-dimethylaminopropyl) carbodiimide (EDCI), O-benzotriazole-N,N,N′,N′-tetramethylurea tetrafluoroborate (TBTU), etc.
c) compound (I-1b) is obtained by reacting berbamine as a raw material with sulfonylation reagent in the presence of a base;
bis-benzyl isoquinoline compound I-1 is obtained by coupling (I-1b) with the coupling reagent, and the reaction is shown in scheme I:

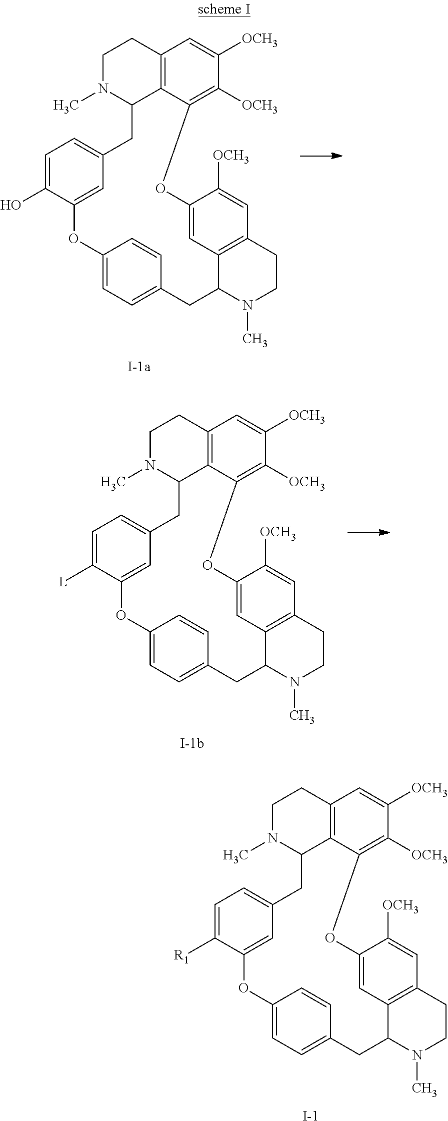

in formula I-1, R₁ is selected from hydrogen, halogen, C₁-C₆ alkylthio, C₁-C₆ alkyl, C₃-C₆ cycloalkyl, C₂-C₆ alkenyl, C₂-C₆ alkynyl, amino substituted by C₁-C₆ alkyl, cyano, carboxy, aldehyde, C₁-C₆ alkanoyl;
L in formula 1-1b is selected from leaving group, such as C₁-C₆ alkylsulfonyloxy, halogenated C₁-C₆ alkylsulfonyloxy, benzenesulfonyloxy, naphthalenesulfonyloxy, preferably methanesulfonyloxy and trifluoromethanesulfonyloxy;
d) using berbamine as a raw material, the compound of formula (I-2) is prepared by nitration, reduction and ring-closing reaction, as shown in the following scheme 2:

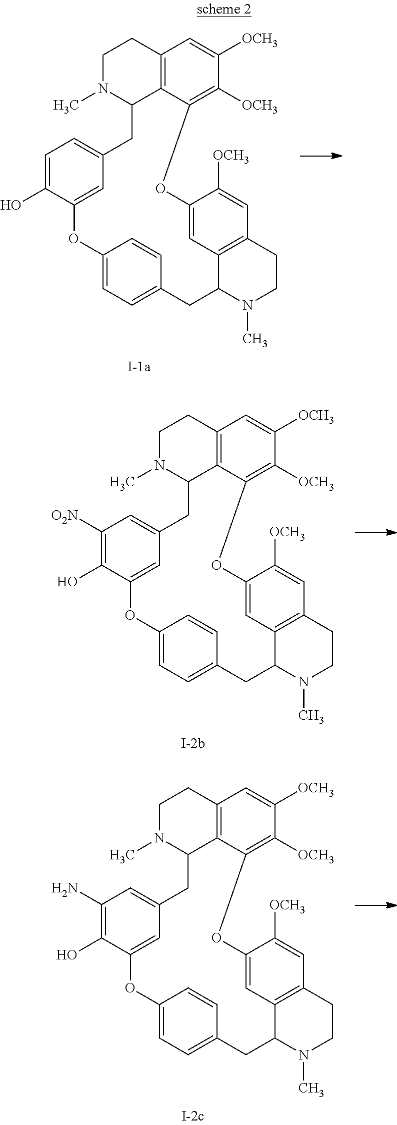

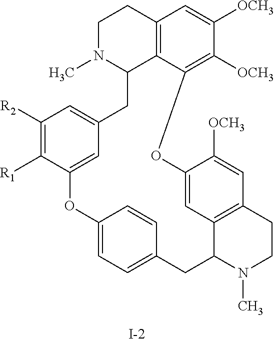

in formula I-2, R₁ and R₂, together with the adjacent benzene ring, form benzo[5-6 membered monocyclic heterocycle] unsubstituted or substituted with 1-4 substituents; In another preferred embodiment, the benzo[5-6 membered monocyclic heterocycle] is selected from:

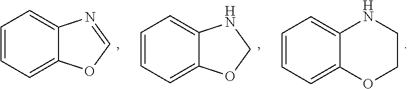

In another preferred embodiment, the substituent is selected from halogen, hydroxy, mercapto, amino, oxo (═O), thio (═S), C₁˜C₆ alkyl; the heterocycle contains 1 to 3 heteroatoms selected from N, O, S;
e) using berbamine as a raw material, the compound of formula (I-3) is prepared by multi-step reaction, as shown in scheme 3:

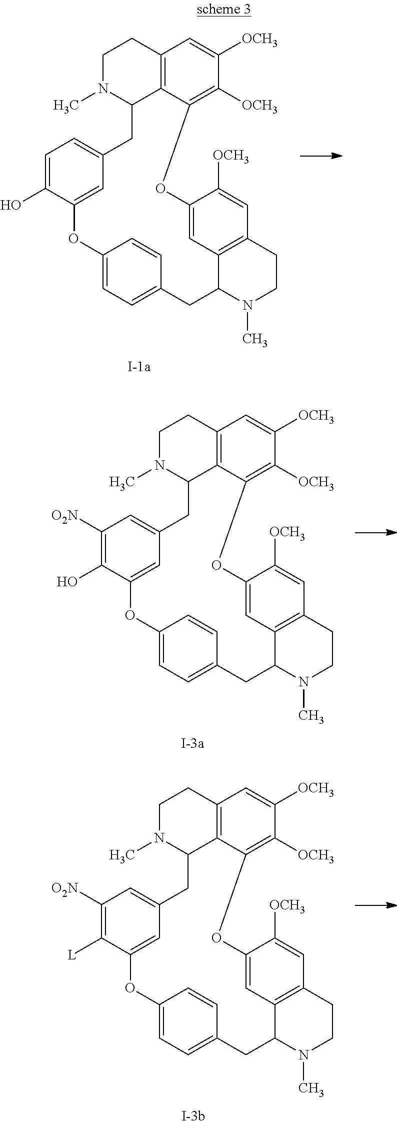

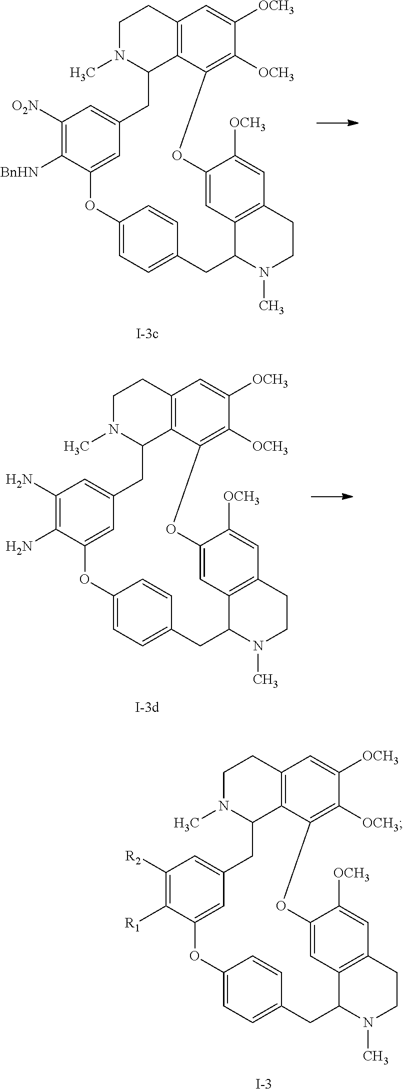

in formula I-3, R₁ and R₂ together with the adjacent benzene ring form benzo[5-6 membered monocyclic heterocycle] unsubstituted or substituted with 1-4 substituents;
the substituent is selected from halogen, hydroxy, mercapto, amino, oxo (═O), thio (═S), C₁˜C₆ alkyl;
the heterocycle contains 1 to 3 heteroatoms selected from N, O, S.

In another preferred embodiment, the benzo[5-6 membered monocyclic heterocycle] is selected from:

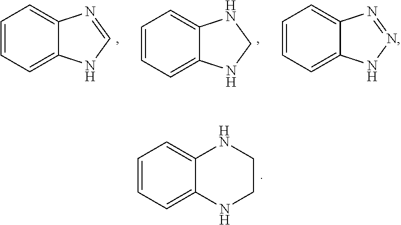

In formula I-3b, L is a leaving group, such as C₁-C₆ alkylsulfonyloxy, halogenated C₁-C₆ alkylsulfonyloxy, benzenesulfonyloxy, naphthalenesulfonyloxy, preferably methanesulfonyloxy and trifluoromethanesulfonyloxy.

In another preferred embodiment, the method e) includes the following steps:
e1) the compound of formula (I-1a) and the nitration reagent undergo an ortho-nitration reaction to generate the compound of formula (I-3a);
e2) the compound of formula (I-3a) is reacted with a sulfonylation reagent in the presence of a base in a suitable solvent to obtain the compound of formula (I-3b);
e3) the compound of formula (I-3b) and benzylamine undergo a Buchwald-Hartig reaction to obtain the compound of formula (I-3c);
e4) the compound of formula (I-3c) undergoes a reduction reaction to obtain the compound of formula (I-3d);
e5) the compound of formula (1-3d) undergoes a ring-closing reaction to obtain the compound of formula (I-3);
f) compound (I-4b) is obtained by reacting Fangchinoline as a raw material with sulfonylation reagent in the presence of a base;
the compound of formula I-4 is obtained by coupling (I-4b) and coupling reagents, as shown in scheme 4:

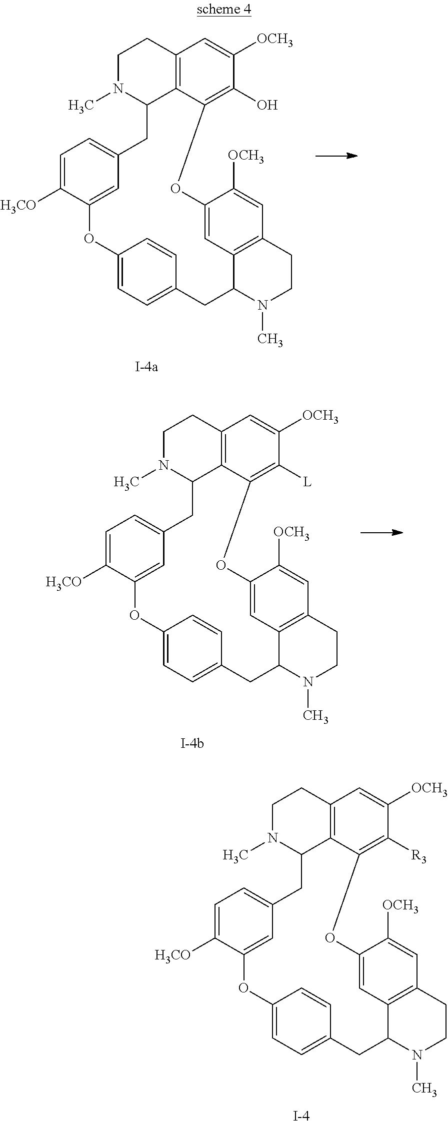

in formula I-4, R₃ is selected from hydrogen, halogen, C₁-C₆ alkylthio, C₁-C₆ alkyl, C₃-C₆ cycloalkyl, C₂-C₆ alkenyl, C₂-C₆ alkynyl, amino substituted by C₁-C₆ alkyl, cyano, carboxy, aldehyde, C₁-C₆ alkanoyl;
g) the compound obtained by any one of methods a) to f) is subjected to functional group transformation to obtain the bisbenzyl isoquinoline compound.

### Pharmaceutical Composition and Application

The present invention also provides a use of one or a mixture of the active compound of formula I for inhibiting coronavirus replication of the present invention, or a pharmaceutically acceptable salt thereof, or a prodrug thereof as an active ingredient in the preparation of a medicament for treating and/or preventing or alleviating respiratory tract infection, pneumonia and other related diseases caused by coronavirus infection such as 2019 novel coronavirus.

The pharmaceutical composition provided by the present invention preferably contains the active ingredient in a weight ratio of 0.001-99 wt %, preferably the active compound of the present invention as the active ingredient accounts for 0.1-90 wt % or 1-50 wt % of the total weight, and the rest is a pharmaceutically acceptable carrier, diluent or solution or salt solution.

When desired, one or more pharmaceutically acceptable carriers may also be added to the medicaments of the present invention. The carrier includes conventional diluents, excipients, fillers, binders, wetting agents, disintegrants, absorption enhancers, surfactants, adsorption carriers, lubricants and the like in the pharmaceutical field.

The compounds and pharmaceutical compositions provided by the present invention can be in various forms, such as tablets, capsules, powders, syrups, solutions, suspensions and aerosols, etc., and can be present in suitable solid or liquid carriers or diluents and sterilization equipment suitable for injection or drip.

Various dosage forms of the pharmaceutical composition of the present invention can be prepared according to conventional preparation methods in the pharmaceutical field. The unit dose of the preparation formula usually contains 0.05-400 mg of the active compound of the present invention, preferably, the unit dose of the preparation formula contains 1 mg-500 mg of the active compound of the present invention.

The compounds and pharmaceutical compositions of the present invention can be used clinically in mammals, including humans and animals, by oral, nasal, dermal, pulmonary or gastrointestinal administration, most preferably by oral. Most preferably, the daily dose is 0.01 to 400 mg/kg body weight in one dose or 0.01 to 200 mg/kg body weight in divided doses. Regardless of the method of administration, the individual's optimal dosage should be determined based on the specific treatment. Normally, start with a small dose and gradually increase the dose until the most suitable dose is found.

The drug or inhibitor of the present invention can be administered in various ways, for example, it can be introduced into the body such as muscle, intradermal, subcutaneous, intravenous, mucosal tissue by injection, spray, intranasal, ocular, osmotic, absorption, physically or chemically mediated methods; or it can be introduced into the body by mixing or wrapping with other substances.

Typically, the active ingredient of the present invention or the pharmaceutical composition containing it can be administered in a unit dosage form, and the administration route can be intestinal or parenteral, such as oral administration, intravenous injection, intramuscular injection, subcutaneous injection, nasal cavity, oral mucosa, eye, lung and respiratory tract, skin, vagina, rectum, etc.

The dosage form may be a liquid dosage form, a solid dosage form, or a semi-solid dosage form. Liquid dosage forms can be solutions (including true solutions and colloidal solutions), emulsions (including o/w, w/o and multiple emulsions), suspensions, injections (including water injections, powder injections and infusions), eye drops, nose drops, lotions and liniments, etc; solid dosage forms can be tablets (including ordinary tablets, enteric-coated tablets, lozenges, dispersible tablets, chewable tablets, effervescent tablets, orally disintegrating tablets), capsules (including hard capsules, soft capsules, enteric capsules), granules, powders, pellets, dropping pills, suppositories, films, patches, aerosol (powder), sprays, etc.; semi-solid dosage forms can be ointments, gels, pastes, etc.

The active ingredient of the present invention can be formulated into a general formulation, and can also be formulated into a sustained-release formulation, a controlled-release formulation, a targeted formulation, and various microparticle drug delivery systems.

In order to form the active ingredient of the present invention into a tablet, various excipients known in the art can be widely used, including diluents, binders, wetting agents, disintegrants, lubricants, glidants. The diluent can be starch, dextrin, sucrose, glucose, lactose, mannitol, sorbitol, xylitol, microcrystalline cellulose, calcium sulfate, calcium hydrogen phosphate, calcium carbonate, etc.; the wetting agent can be water, ethanol, isopropanol, etc; the binder can be starch slurry, dextrin, syrup, honey, glucose solution, microcrystalline cellulose, arabic mucilage, gelatin pulp, sodium carboxymethyl cellulose, methyl cellulose, hydroxypropyl methyl cellulose, ethyl cellulose, acrylic resin, carbomer, polyvinylpyrrolidone, polyethylene glycol, etc.; the disintegrants can be dry starch, microcrystalline cellulose, low-substituted hydroxypropyl cellulose, cross-linked polyvinylpyrrolidone, cross-linked sodium carboxymethyl cellulose, sodium carboxymethyl starch, sodium bicarbonate and citric acid, polyoxyethylene sorbitol fatty acid ester, sodium dodecyl sulfonate etc.; the lubricants and glidants may be talc, silica, stearates, tartaric acid, liquid paraffin, polyethylene glycol, and the like.

The tablet may be further formed into a coated tablet, such as a sugar-coated tablet, a film-coated tablet, an enteric-coated tablet, or a double-layered tablet and a multi-layered tablet.

In order to form the administration unit into a capsule, the active ingredient of the present invention can be mixed with a diluent and a glidant, and the mixture can be directly placed in a hard capsule or a soft capsule. The active ingredient can also be made into granules or pellets with diluents, binders and disintegrating agents, and then placed in hard or soft capsules. The types of diluents, binders, wetting agents, disintegrants, and glidants used to prepare the tablets of the present invention can also be used to prepare the capsules of the present invention.

In order to prepare the active ingredient of the present invention into an injection, water, ethanol, isopropanol, propylene glycol or a mixture thereof can be used as a solvent, and an appropriate amount of solubilizer, cosolvent, pH regulator and osmotic pressure regulator commonly used in the art can be added. The solubilizer or cosolvent can be poloxamer, lecithin, hydroxypropyl-β-cyclodextrin, etc.; the pH regulator can be phosphate, acetate, hydrochloric acid, sodium hydroxide, etc.; the osmotic pressure regulator can be sodium chloride, mannitol, glucose, phosphate, acetate, etc. If freeze-dried powder injection is prepared, mannitol, glucose and so on can also be added as proppant.

In addition, coloring agents, preservatives, perfumes, flavoring agents, or other additives may also be added to the pharmaceutical preparation, if desired.

The active ingredient or composition of the present invention may be administered alone or in combination with other therapeutic or symptomatic drugs.

When there is a synergistic effect between the active ingredient of the present invention and other therapeutic drugs, its dosage should be adjusted according to the actual situation.

### The Main Advantages of the Invention Include

1) The compounds of the present invention have good inhibitory activity on coronavirus replication; the EC₅₀ of some compounds inhibiting the RNA replication of the new coronavirus even reaches the level of <1 μM.

2) The compound of the present invention does not contain phenolic hydroxy group, which not only significantly improves the anti-coronavirus SARS-CoV-2, but also has good physical and chemical properties, good metabolic properties and high oral bioavailability.

3) The compound of the present invention not only has strong activity and high therapeutic index SI, so it is suitable for effective oral administration with the characteristics of low efficacy dose, small toxic side effects, etc., can be used to prevent and/or treat related diseases caused by coronavirus infection, and has good clinical application prospects.

The present invention will be further illustrated below with reference to the specific examples. It should be understood that these examples are only to illustrate the invention but not to limit the scope of the invention. In the following examples, the experimental methods without specific conditions are usually in accordance with conventional conditions, such as Sambrook et al., Molecular Cloning: Laboratory Manual (New York: Cold Spring Harbor Laboratory Press, 1989), or in accordance with the conditions recommended by the manufacturer. Unless otherwise stated, percentages and parts are percentages by weight and parts by weight.

### Example 1 Preparation of Hydrochloride Salt of Compound 1

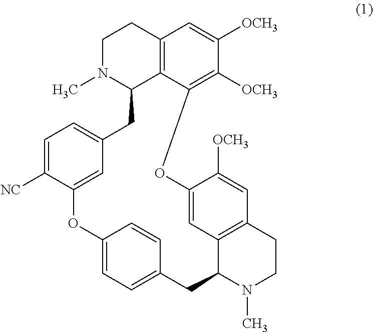 [SMILES: COc1cc2c3cc1Oc1c(OC)c(OC)cc4c1[C@@H](Cc1ccc(C#N)c(c1)Oc1ccc(cc1)C[C@@H]3N(C)CC2)N(C)CC4]

The steps were as follows:

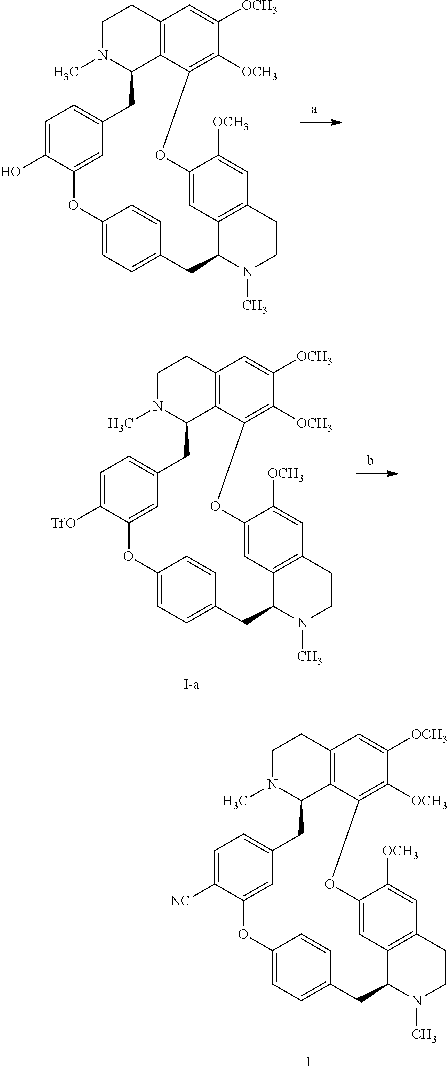 [SMILES: COc1cc2c3cc1Oc1c(OC)c(OC)cc4c1[C@@H](Cc1ccc(O)c(c1)Oc1ccc(cc1)C[C@@H]3N(C)CC2)N(C)CC4 ; COc1cc2c3cc1Oc1c(OC)c(OC)cc4c1[C@@H](Cc1ccc(OS(=O)(=O)C(F)(F)F)c(c1)Oc1ccc(cc1)C[C@@H]3N(C)CC2)N(C)CC4 ; COc1cc2c3cc1Oc1c(OC)c(OC)cc4c1[C@@H](Cc1ccc(C#N)c(c1)Oc1ccc(cc1)C[C@@H]3N(C)CC2)N(C)CC4]

### Step 1

Berbamine (185 mg, 0.304 mmol), DCM(2 mL) and pyridine (0.122 mL, 1.52 mmol, 5 eq) were added into a 10 mL single-necked flask, stirred in ice bath for 5 min, Tf₂O (0.1 mL, 0.61 mmol, 2 eq) was added dropwise, and stirred at room temperature for 30 min after the addition. TLC detection showed that the reaction was completed. 1 N hydrochloric acid (1.5 mL) was added, and stirred, and the organic layer was separated. The organic layer was washed with sodium bicarbonate aqueous solution once again, dried over anhydrous sodium sulfate, concentrated, and purified by silica gel column chromatography to give 1-a (180 mg, 0.243 mmol) as a pale yellow solid, yield 80%.

¹H NMR (500 MHZ, Chloroform-d) δ 7.47 (dd, J=8.5, 2.2 Hz, 1H), 7.07 (d, J=8.3 Hz, 1H), 6.97 (td, J=8.8, 2.4 Hz, 2H), 6.83 (dd, J=8.4, 2.0 Hz, 1H), 6.61 (s, 1H), 6.37 (s, 1H), 6.34-6.32 (m, 1H), 6.31 (s, 1H), 5.60 (d, J=2.0 Hz, 1H), 4.20 (d, J=5.9 Hz, 1H), 3.79 (s, 3H), 3.66 (d, J=3.4 Hz, 1H), 3.63 (s, 3H), 3.38 (d, J=14.4 Hz, 1H), 3.24-3.14 (m, 5H), 3.05 (m, 1H), 2.94 (m, 1H), 2.83 (m, 2H), 2.73 (m, 2H), 2.67 (s, 3H), 2.56 (s, 3H), 2.41-2.29 (m, 3H). ESI-MS (m/z): 741.4 [M+H]⁺.

### Step 2

1-a (170 mg, 0.230 mmol), zinc cyanide (40 mg, 0.345 mmol, 1.5 eq), Pd₂(dba)₃ (11 mg, 0.012 mmol, 0.05 eq), DPPF (13 mg, 0.023 mmol, 0.1 eq) and zinc powder (2 mg, 0.03 mmol, 0.13 eq) were mixed in DMAC (2 mL) and replaced with nitrogen for three times, and the mixture was heated to 150° C. and reacted for 3 h. After the reaction solution was cooled to room temperature, EA was added to dilute, the insoluble substance was filtered out, the filter residue was washed with EA, and the filtrate was washed with water twice, dried over anhydrous sodium sulfate, concentrated, and purified by silica gel column chromatography to obtain basic compound 1 (110 mg, 0.178 mmol) as a pale yellow oil, yield 78%.

¹H NMR (500 MHZ, Chloroform-d) δ 7.49 (dd, J=8.4, 2.3 Hz, 1H), 7.39 (d, J=7.9 Hz, 1H), 6.99 (dd, J=8.2, 2.3 Hz, 1H), 6.95 (dd, J=8.3, 2.6 Hz, 1H), 6.87 (dd, J=7.9, 1.4 Hz, 1H), 6.60 (s, 1H), 6.37 (s, 1H), 6.33 (dd, J=8.3, 2.6 Hz, 1H), 6.31 (s, 1H), 5.55 (d, J=1.4 Hz, 1H), 4.22 (d, J=6.0 Hz, 1H), 3.79 (s, 3H), 3.69 (d, J=3.8 Hz, 1H), 3.64 (s, 3H), 3.44-3.39 (m, 1H), 3.28-3.14 (m, 5H), 3.06 (m, 1H), 2.96 (m, 1H), 2.88-2.79 (m, 2H), 2.78-2.70 (m, 2H), 2.68 (s, 3H), 2.56 (s, 3H), 2.40-2.27 (m, 3H).

105 mg of basic compound 1 was dissolved in a small amount of methanol, 0.051 mL of concentrated hydrochloric acid was added, stirred at room temperature for 5 min, concentrated to remove the solvent, acetonitrile was added to the residual liquid, spin dried again to obtain some solids, appropriate amount of methyl tertiary ether was added to slurry, filtered, and the filter cake was dried to obtain dihydrochloride of the compound 1 (87 mg) as a yellow solid.

ESI-MS (m/z): 618.4 [M+H]⁺. HPLC purity>99%.

### Example 2 Preparation of Compound 2

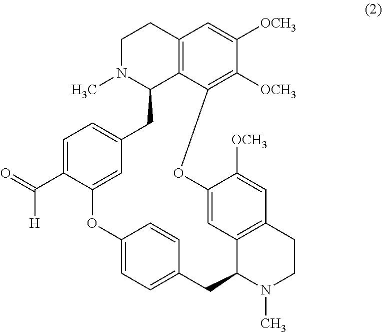 [SMILES: COc1cc2c3cc1Oc1c(OC)c(OC)cc4c1[C@@H](Cc1ccc(C=O)c(c1)Oc1ccc(cc1)C[C@@H]3N(C)CC2)N(C)CC4]

The product of Example 1 (75 mg, 0.108 mmol) was taken and dissolved in formic acid (3 mL), Raney Ni (200 mg) was added, replaced with nitrogen, reacted at 90° C. for 2 h, insoluble substance was filtered out, the filter residue was washed with methanol, and the filtrate was concentrated to dryness, diluted with DCM, washed with sodium bicarbonate, dried over anhydrous sodium sulfate, concentrated, and purified by silica gel column chromatography to obtain title Compound 2 (38 mg, 0.061 mmol) as a yellow oil, yield 57%.

ESI-MS (m/z): 621.4 [M+H]⁺.

### Example 3 Preparation of Hydrochloride Salt of Compound 3

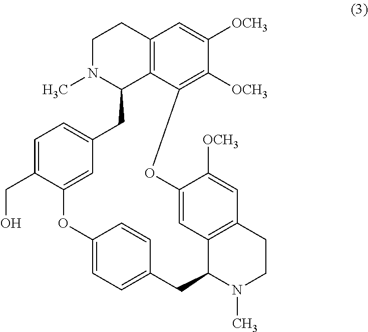 [SMILES: COc1cc2c3cc1Oc1c(OC)c(OC)cc4c1[C@@H](Cc1ccc(CO)c(c1)Oc1ccc(cc1)C[C@@H]3N(C)CC2)N(C)CC4]

The product of Example 2 (36 mg, 0.058 mmol) was taken and dissolved in 1 mL methanol, sodium borohydride (4 mg, 0.106 mmol, 1.8 eq) was added under ice bath, slowly warmed to room temperature, saturated aqueous ammonium chloride solution was added to quench the reaction after 2 h, DCM and water were added, and the organic layer was separated, dried over anhydrous sodium sulfate, concentrated, and purified by silica gel column chromatography to obtain basic title compound (20 mg, 0.032 mmol) as a light yellow solid, yield 55%. Due to the presence of atropisomerism, the nuclear magnetic spectrum showed two groups of peaks, and the ratio thereof is about 3:2.

Main peak ¹H NMR (600 MHz, Chloroform-d) δ 7.80 (brs, 1H), 7.37 (d J=8.4 Hz, 1H), 7.21 (d J=7.3 Hz, 1H), 6.92 (m, 2H), 6.68 (s, 1H), 6.44 (s, 1H), 6.37 (s, 1H), 6.32 (dd J=8.1, 2.6 Hz, 1H), 5.32 (s, 1H), 4.78 (m, 2H), 4.59 (m, 1H), 4.14-3.85 (m, 2H), 3.82 (s, 3H), 3.78-3.46 (m, 2H), 3.65 (s, 3H), 3.36-3.13 (m, 3H), 3.22 (s, 3H), 3.08-2.91 (m, 3H), 2.90-2.67 (m, 3H), 2.82 (s, 3H), 2.73 (s, 3H); secondary peak ¹H NMR (600 MHz, Chloroform-d) δ 7.47 (d J=7.8 Hz, 1H), 7.43 (brs, 1H), 7.16 (brs, 1H), 6.92 (m, 2H), 6.61 (dd J=8.5, 2.7 Hz, 1H), 6.48 (s, 1H), 6.47 (s, 1H), 6.44 (s, 1H), 5.32 (s, 1H), 4.78 (m, 2H), 4.75 (m, 1H), 4.14-3.85 (m, 2H), 3.83 (s, 3H), 3.78-3.46 (m, 2H), 3.43 (s, 3H), 3.36-3.13 (m, 3H), 3.18 (s, 3H), 3.08-2.91 (m, 3H), 2.90-2.67 (m, 3H), 2.79 (s, 3H), 2.69 (s, 3H).

12 mg of basic compound 3 was taken and dissolved in a small amount of methanol, 3.5 μL of concentrated hydrochloric acid was added, stirred at room temperature for 5 min, concentrated to remove the solvent, acetonitrile was added to the residual liquid, and solid was precipitated, slurried, filtered and dried to obtain dihydrochloride of the title compound 3 (13 mg) as an off-white solid.

ESI-MS (m/z): 623.4 [M+H]⁺. HPLC purity>99%.

### Example 4 Preparation of Compound 4

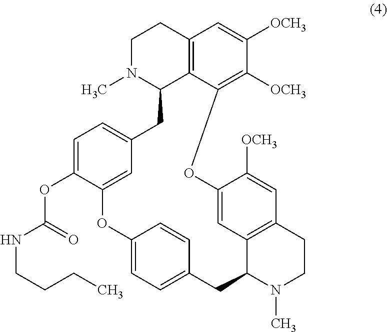 [SMILES: CCCCNC(=O)Oc1ccc2cc1Oc1ccc(cc1)C[C@H]1c3cc(c(OC)cc3CCN1C)Oc1c(OC)c(OC)cc3c1[C@@H](C2)N(C)CC3]

100 mg of berbamine hydrochloride, 53 mg of triethylamine, 22 mg of n-butyl isocyanate and 3 mL of acetonitrile were added into a 10 mL three-necked flask, after stirring at 25° C. for 2 hours, the reaction solution was concentrated to dryness, purified by silica gel column chromatography (SiO₂, dichloromethane:methanol=30:1) to afford 72 mg of the product as a pale yellow solid, yield 67.8%. Due to the presence of atropisomerism, the nuclear magnetic spectrum showed two groups of peaks, and the ratio thereof is about 2:1.

¹H NMR (500 MHZ, Methanol-d₄, major rotamer) δ 7.45 (dd, J=8.4, 2.3 Hz, 1H), 7.13 (m, 1H), 6.98 (dd, J=7.9, 2.1 Hz, 1H), 6.94 (d, J=8.1 Hz, 1H), 6.89 (m, 1H), 6.76 (s, 1H), 6.53 (s, 1H), 6.52 (s, 1H), 6.37 (dd, J=8.2, 2.6 Hz, 1H), 5.53 (d, J=1.9 Hz, 1H), 4.34 (d, J=5.9 Hz, 1H), 3.92 (m, 1H), 3.80 (s, 3H), 3.64 (s, 3H), 3.37 (m, 1H), 3.24 (m, 1H), 3.19 (s, 3H), 3.17 (m, 3H), 3.06 (m, 2H), 2.92-2.76 (m, 4H), 2.72-2.42 (m, 3H), 2.67 (s, 3H), 2.65 (s, 3H), 1.49 (m, 2H), 1.33 (m, 2H), 0.89 (t, J=7.3 Hz, 3H); ¹H NMR (500 MHz, Methanol-d₄, minor rotamer) δ 7.35 (dd, J=8.5, 2.3 Hz, 1H), 7.19 (d, J=8.2 Hz, 1H), 6.89 (m, 2H), 6.83 (dd, J=8.4, 2.7 Hz, 1H), 6.69 (s, 1H), 6.59 (s, 1H), 6.56 (s, 1H), 6.39 (m, 1H), 4.50 (dd, J=11.5, 4.2 Hz, 1H), 3.80 (m, 4H), 3.67 (m, 1H), 3.49 (d, J=7.3 Hz, 1H), 3.40 (s, 3H), 3.24 (m, 1H), 3.17 (m, 3H), 3.10 (s, 3H), 3.06 (m, 2H), 2.92-2.76 (m, 4H), 2.72-2.42 (m, 3H), 2.66 (s, 3H), 2.61 (s, 3H), 1.49 (m, 2H), 1.33 (m, 2H), 0.86 (t, J=7.3 Hz, 3H).

### Example 5 Preparation of Compound 5

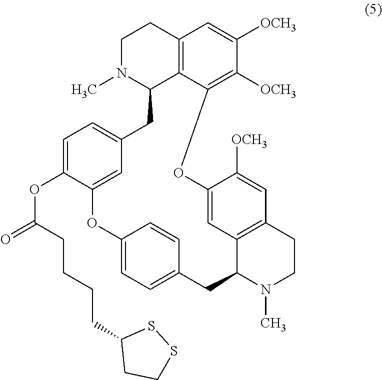 [SMILES: COc1cc2c3cc1Oc1c(OC)c(OC)cc4c1[C@@H](Cc1ccc(OC(=O)CCCC[C@H]5CCSS5)c(c1)Oc1ccc(cc1)C[C@@H]3N(C)CC2)N(C)CC4]

100 mg of berbamine hydrochloride, 31 mg of S-lipoic acid, 43 mg of EDCI, 4 mg of DMAP and 3 mL of dichloromethane were added to a 10 mL three-necked flask and stirred at 25° C. for 5 hours. The reaction solution was added with 2 mL of water to layer, the organic phase was added with 2 mL of saturated brine to layer, and the organic phase was dried over anhydrous sodium sulfate. The solvent was distilled off under reduced pressure to obtain the crude product, which was finally purified by column chromatography (SiO₂, dichloromethane:methanol=50:1) to obtain 63 mg of the title compound as a pale yellow solid product, yield 52.5%. Due to the presence of atropisomerism, the nuclear magnetic spectrum showed two groups of peaks, and the ratio thereof is about 5:2.

¹H NMR (500 MHZ, Methanol-d₄, major rotamer) δ 7.45 (dd, J=8.5, 2.2 Hz, 1H), 7.16 (m, 1H), 6.99 (dd, J=8.3, 2.2 Hz, 1H), 6.90 (m, 2H), 6.74 (s, 1H), 6.51 (s, 1H×2), 6.34 (dd, J=8.2, 2.6 Hz, 1H), 5.59 (d, J=1.7 Hz, 1H), 4.25 (d, J=5.7 Hz, 1H), 3.80 (s, 3H), 3.75 (d, J=3.7 Hz, 1H), 3.64 (s, 3H), 3.46 (m, 1H), 3.24-3.16 (m, 2H), 3.20 (s, 3H), 3.10-3.04 (m, 2H), 3.03-2.96 (m, 2H), 2.93 (m, 1H), 2.82-2.70 (m, 3H), 2.63-2.37 (m, 5H), 2.60 (s, 3H), 2.57 (s, 3H), 2.32 (m, 1H), 1.80-1.48 (m, 8H); ¹H NMR (500 MHZ, Methanol-d₄, minor rotamer) δ 7.34 (dd, J=8.5, 2.2 Hz, 1H), 7.16 (m, 1H), 6.90 (m, 2H), 6.79 (dd, J=8.4, 2.7 Hz, 1H), 6.69 (s, 1H), 6.55 (s, 1H), 6.54 (s, 1H), 6.37 (m, 1H), 4.31 (dd, J=11.4, 4.2 Hz, 1H), 3.80 (m, 4H), 3.46 (m, 2H), 3.40 (s, 3H), 3.24-3.16 (m, 2H), 3.13 (s, 3H), 3.10-3.04 (m, 2H), 3.03-2.96 (m, 2H), 2.93 (m, 1H), 2.82-2.70 (m, 3H), 2.63-2.37 (m, 5H), 2.59 (s, 3H), 2.52 (s, 3H), 2.32 (m, 1H), 1.80-1.48 (m, 8H). LRMS: m/z 797.5 [M+H]⁺, m/z 399.5 [M/2+H]⁺.

### Example 6 Preparation of Compound 6 Hydrochloride

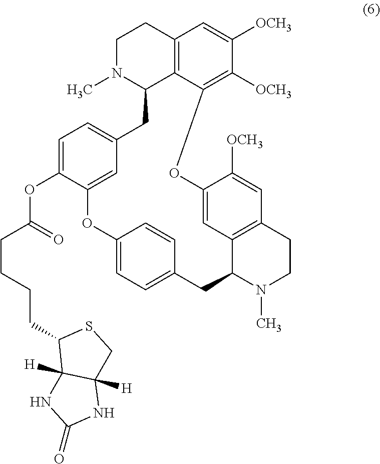 [SMILES: COc1cc2c3cc1Oc1c(OC)c(OC)cc4c1[C@@H](Cc1ccc(OC(=O)CCCC[C@@H]5SC[C@@H]6NC(=O)N[C@@H]65)c(c1)Oc1ccc(cc1)C[C@@H]3N(C)CC2)N(C)CC4]

Berbamine (60 mg, 0.099 mmol) and D-Biotin (29 mg, 0.119 mmol, 1.2 eq) were suspended in DCM (1 mL). EDCI hydrochloride (29 mg, 0.151 mmol, 1.5 eq) and DMAP (4 mg, 0.033 mmol, 0.3 eq) were added successively at room temperature. After the addition, the mixture was reacted overnight at room temperature. The mixture was diluted with DCM, washed with saturated aqueous ammonium chloride solution, washed with brine, and the organic layer was separated, dried over anhydrous sodium sulfate, concentrated, and separated by silica gel column chromatography to obtain basic title compound as an off-white solid, 58 mg, yield 70%. Due to the presence of atropisomerism, the nuclear magnetic spectrum showed two groups of peaks, and the ratio thereof is about 2:1.

Peak of major rotamer ¹H NMR (600 MHZ, Chloroform-d) δ 7.63 (brs, 1H), 7.17 (m, 1H), 6.97 (dd J=8.4, 2.6 Hz, 1H), 6.91 (m, 2H), 6.63 (s, 1H), 6.43 (s, 1H), 6.41 (s, 1H), 6.39 (s, 1H), 6.34 (s, 1H), 6.31 (dd J=8.2, 2.6 Hz, 1H), 5.54 (d J=1.6 Hz, 1H), 4.85 (s, 1H), 4.67 (brs, 1H), 4.35 (m, 2H), 3.96 (m, 1H), 3.80 (s, 3H), 3.62 (s, 3H), 3.62 (m, 1H), 3.53-3.23 (m, 2H), 3.20 (s, 3H), 3.12-3.02 (m, 2H), 3.02-2.76 (m, 6H), 2.76-2.36 (m, 5H), 2.75 (s, 3H), 2.60 (s, 3H), 1.76 (m, 2H), 1.61 (m, 2H), 1.47 (m, 2H).

50 mg of basic compound 6 was taken and dissolved in a small amount of methanol, 12.5 μL concentrated hydrochloric acid was added, stirred at room temperature for 5 min, concentrated to remove the solvent, acetonitrile was added to the residue, concentrated to remove the solvent again, appropriate amount of acetone was added to slurry, filtered and dried to obtain title compound 38 mg (dihydrochloride form) as an off-white solid. ESI-MS (m/z): 835.5 [M+H]⁺.

### Example 7 Preparation of Compound 7

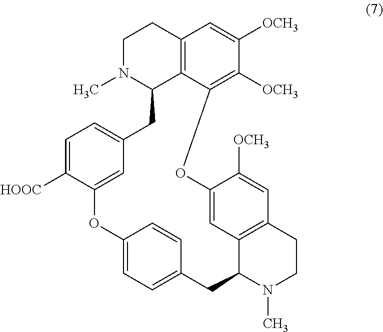 [SMILES: COc1cc2c3cc1Oc1c(OC)c(OC)cc4c1[C@@H](Cc1ccc(C(=O)O)c(c1)Oc1ccc(cc1)C[C@@H]3N(C)CC2)N(C)CC4]

Example 7 was prepared using a method similar to Example 9 (see below).

Compound 1 (470 mg, 0.681 mmol) was suspended in ethylene glycol (5 mL), potassium hydroxide (213 mg, 3.80 mmol, 5.6 eq) and water (0.32 mL) were added, replaced with nitrogen, and heated to 150° C. and reacted for 6 h. After the reaction solution was cooled to room temperature, water (10 mL) was added to dilute, the pH was adjusted to weak alkalinity with dilute hydrochloric acid, DCM was added to extract, and the organic phase was washed with water once, brine once, dried over anhydrous sodium sulfate, concentrated, and purified by silica gel column chromatography to obtain basic compound 7 (120 mg) as a light yellow solid, yield 28%. Due to the presence of atropisomerism, the nuclear magnetic spectrum showed two groups of peaks, and the ratio thereof is about 5:4.

Peak of major rotamer ¹H NMR (600 MHZ, Chloroform-d) δ 7.67 (d J=7.9 Hz, 1H), 7.48 (d J=8.4 Hz, 1H), 6.92 (m, 1H), 6.80 (m, 1H), 6.60 (s, 1H), 6.57 (m, 1H), 6.38 (s, 1H), 6.22 (s, 1H), 6.06 (d J=8.3 Hz, 1H), 5.52 (s, 1H), 4.45 (d J=5.8 Hz, 1H), 3.98 (m, 1H), 3.79 (s, 3H), 3.59-3.39 (m, 2H), 3.52 (s, 3H), 3.36-3.20 (m, 2H), 3.19 (s, 3H), 3.14-3.00 (m, 2H), 2.90-2.62 (m, 4H), 2.77 (s, 3H), 2.58 (s, 3H), 2.43-2.25 (m, 2H). Peak of minor rotamer ¹H NMR (600 MHz, Chloroform-d) δ 7.78 (d J=8.0 Hz, 1H), 7.20 (d J=8.0 Hz, 1H), 6.92 (m, 1H), 6.87 (m, 1H), 6.72 (dd J=8.2, 2.7 Hz, 1H), 6.62 (s, 1H), 6.38 (s, 1H), 6.35 (s, 1H), 6.30 (m, 1H), 6.12 (m, 1H), 4.59 (dd J=12.0, 3.3 Hz, 1H), 3.77 (s, 3H), 3.70 (m, 1H), 3.59-3.39 (m, 2H), 3.36-3.20 (m, 2H), 3.34 (s, 3H), 3.14-3.00 (m, 2H), 3.12 (s, 3H), 2.90-2.62 (m, 4H), 2.76 (s, 3H), 2.59 (s, 3H), 2.43-2.25 (m, 2H). ESI-MS (m/z): 637.4 [M+H]⁺, 635.3 [M−H]⁻.

40 mg of basic compound 7 was taken into methanol to prepare hydrochloride salt (concentrated hydrochloric acid 6 μL), spin dried, co-boiled with acetonitrile, slurried in MTBE, filtered and dried to obtain 26 mg of hydrochloride of the compound 7 as an off-white solid. ESI-MS (m/z): 637.4 [M+H]⁺, 635.3 [M−H]⁻.

### Example 8 Preparation of Compound 8

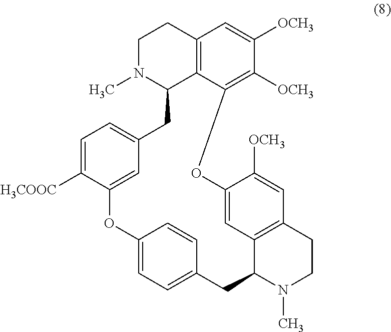

Compound 8 was prepared by esterification using compound 7 prepared in Example 7. Compound 7 (120 mg, 0.189 mmol) was dissolved in methanol (2 mL), concentrated sulfuric acid (78 mg, 0.796 mmol, 4.2 eq) was added at room temperature, and reacted for 5 h at reflux after the addition. TLC detection showed that raw materials were converted completely, sodium bicarbonate (200 mg, 2.388 mmol, 12.6 eq) was added, stirred for 5 min, DCM and water were added, and the organic phase was separated, washed with brine, dried over anhydrous sodium sulfate, concentrated, and purified by silica gel column chromatography to obtain basic compound 8 (90 mg) as an off-white solid, yield 73%. Due to the presence of atropisomerism, the nuclear magnetic spectrum showed two groups of peaks, and the ratio thereof is about 4:1.

Peak of major rotamer ¹H NMR (600 MHZ, Chloroform-d) δ 7.70 (d, J=8.0 Hz, 1H), 7.48 (brs, 1H), 6.96 (m, 2H), 6.89 (d, J=7.9 Hz, 1H), 6.61 (s, 1H), 6.37 (s, 1H), 6.32 (dd, J=8.2, 2.6 Hz, 1H), 6.30 (s, 1H), 5.59 (m, 1H), 4.25 (br s, 1H), 3.87 (s, 3H), 3.79 (s, 3H), 3.70 (m, 1H), 3.63 (s, 3H), 3.47-3.20 (m, 3H), 3.18 (s, 3H), 3.10-2.95 (m, 2H), 2.88-2.65 (m, 4H), 2.69 (s, 3H), 2.58 (s, 3H), 2.46-2.22 (m, 3H). Peak of minor rotamer ¹H NMR (600 MHz, Chloroform-d) δ 7.91 (d, J=8.1 Hz, 1H), 7.33 (d, J=8.4 Hz, 1H), 7.16 (m, 1H), 7.12 (s, 1H), 6.96 (m, 1H), 6.66 (dd, J=8.4 Hz, 2.7 Hz, 1H), 6.64 (s, 1H), 6.40 (m, 3H), 4.43 (br s, 1H), 3.89 (s, 3H), 3.81 (s, 3H), 3.47-3.20 (m, 3H), 3.40 (s, 3H), 3.16 (s, 3H), 3.10-2.95 (m, 3H), 2.88-2.65 (m, 7H), 2.60 (s, 3H), 2.46-2.22 (m, 3H), ESI-MS (m/z): 651.5 [M+H]⁺.

22 mg of basic compound 8 was taken into methanol to prepare hydrochloride salt, 7 μL concentrated hydrochloric acid was added, spin dried, co-boiled with acetonitrile, slurried in MTBE, filtered and dried to obtain 15 mg of dihydrochloride of the compound 8 as a light yellow solid.

### Example 9 Preparation of Compound 9

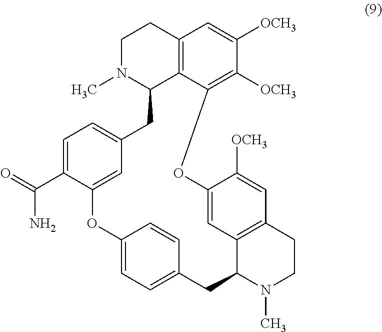 [SMILES: COc1cc2c3cc1Oc1c(OC)c(OC)cc4c1[C@@H](Cc1ccc(C(N)=O)c(c1)Oc1ccc(cc1)C[C@@H]3N(C)CC2)N(C)CC4]

Compound 1 (470 mg, 0.681 mmol) was suspended in ethylene glycol (5 mL), potassium hydroxide (213 mg, 3.80 mmol, 5.6 eq) and water (0.32 mL) were added, replaced with nitrogen, and heated to 150° C. and reacted for 6 h. After the reaction solution was cooled to room temperature, water (10 mL) was added for dilution, dilute hydrochloric acid was added to adjust the pH to weak alkalinity, DCM was added for extraction, and the organic phase was washed with water once, the mixture was washed with brine once, dried over anhydrous sodium sulfate, concentrated, and purified by silica gel column chromatography to give basic compound 9 (70 mg) as a light yellow solid, yield 16%.

¹H NMR (600 MHZ, Chloroform-d) δ 8.03 (d, J=8.0 Hz, 1H), 7.70 (m, 1H), 7.53 (d, J=8.4 Hz, 1H), 6.99 (dd, J=8.2, 2.5 Hz, 3H), 6.61 (s, 1H), 6.37 (s, 1H), 6.32 (s, 1H), 6.31 (m, 1H), 5.70 (m, 1H), 5.61 (d, J=1.5 Hz, 1H), 4.22 (brs, 1H), 3.79 (s, 3H), 3.69 (brs, 1H), 3.64 (s, 3H), 3.40 (m, 1H), 3.33-3.20 (m, 2H), 3.19 (s, 3H), 3.10-2.92 (m, 2H), 2.88-2.84 (m, 2H), 2.73 (m, 2H), 2.69 (s, 3H), 2.58 (s, 3H), 2.41-2.30 (m, 3H). ESI-MS (m/z): 636.4 [M+H]⁺.

### Example 10 Preparation of Compound 10

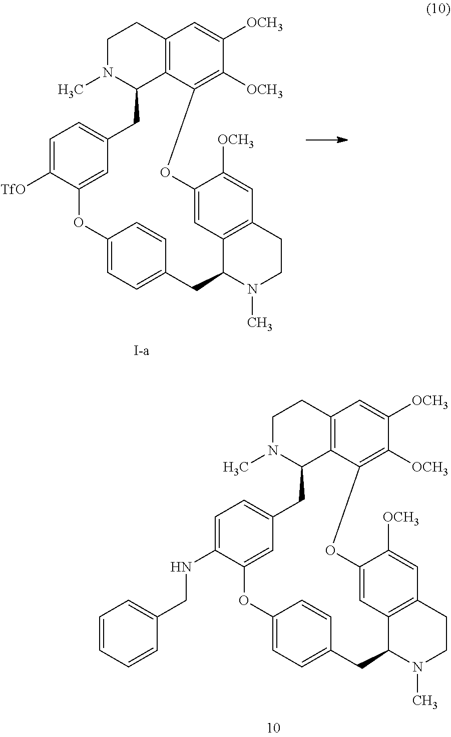 [SMILES: COc1cc2c3cc1Oc1c(OC)c(OC)cc4c1[C@@H](Cc1ccc(OS(=O)(=O)C(F)(F)F)c(c1)Oc1ccc(cc1)C[C@@H]3N(C)CC2)N(C)CC4 ; COc1cc2c3cc1Oc1c(OC)c(OC)cc4c1[C@@H](Cc1ccc(NCc5ccccc5)c(c1)Oc1ccc(cc1)C[C@@H]3N(C)CC2)N(C)CC4]

1-a (500 mg, 0.676 mmol), palladium acetate (24 mg, 0.107 mmol, 0.16 eq), X-Phos(97 mg, 0.203 mmol, 0.3 mmol), cesium carbonate (660 mg, 2.026 mmol, 3 eq), and benzylamine (148 μL, 1.355 mmol, 2 eq) were mixed in dioxane (10 mL), replaced with nitrogen for three times, and the mixture was gradually raised to 102° C. and kept for 12 hours. TLC showed that the raw materials were reacted completely. After the reaction solution was cooled to room temperature, diatomite was used for aid filtration, and the filter residue was washed with ethyl acetate. The filtrate was spin-dried, and purified by silica gel column chromatography (DCM:MeOH:ammonia water=500:10:1.5) to obtain basic compound 10 (420 mg) as a light yellow solid, yield 89%.

ESI-MS (m/z): 698.5 [M+H]⁺.

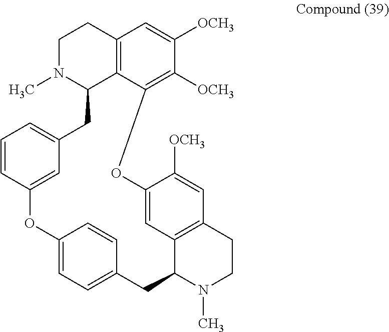 [SMILES: COc1cc2c3cc1Oc1c(OC)c(OC)cc4c1[C@@H](Cc1cccc(c1)Oc1ccc(cc1)C[C@@H]3N(C)CC2)N(C)CC4]

By-product basic compound 39 (40 mg) was also obtained by silica gel column chromatography as a light yellow solid, yield 10%. ESI-MS (m/z): 593.5 [M+⁺H]. 33 mg of basic compound 39 was taken into methanol to prepare hydrochloride salt, 12 μL of concentrated hydrochloric acid was added, spin dried, co-boiled with acetonitrile, slurried in MTBE, filtered and dried to obtain 30 mg of dihydrochloride of compound 39 as a light yellow solid.

### Example 11 Preparation of Compound 11

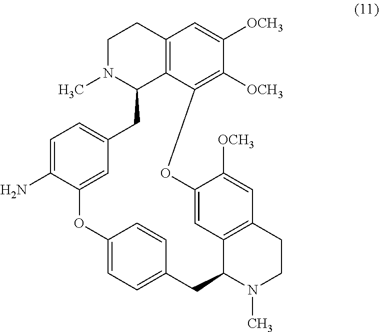 [SMILES: COc1cc2c3cc1Oc1c(OC)c(OC)cc4c1[C@@H](Cc1ccc(N)c(c1)Oc1ccc(cc1)C[C@@H]3N(C)CC2)N(C)CC4]

Compound 10 (380 mg, 0.544 mmol), palladium carbon (40 mg) and ammonium formate (346 mg, 5.48 mmol, 10 eq) were mixed in methanol (5 mL) and refluxed for 5 hours. TLC showed that raw material was completely reacted. Diatomite was used for aid filtration, the filter residue was washed with ethyl acetate, the filtrate was spin-dried, and purified by silica gel column chromatography to obtain basic compound 11 (240 mg) as an off-white solid, yield 73%.

¹H NMR (500 MHZ, Chloroform-d) δ 7.42 (dd, J=8.3, 2.3 Hz, 1H), 6.94 (m, 2H), 6.67 (m, 1H), 6.63 (s, 1H), 6.38 (m, 1H), 6.35 (s, 1H), 6.33 (dd, J=8.2, 2.5 Hz, 1H), 6.30 (s, 1H), 5.38 (d, J=1.8 Hz, 1H), 5.29 (s, 2H), 4.19 (d, J=5.6 Hz, 1H), 3.90 (brs, 1H), 3.78 (s, 3H), 3.74 (m, 1H), 3.62 (s, 3H), 3.61 (m, 1H), 3.32 (m, 1H), 3.18 (s, 3H), 3.11 (m, 1H), 3.07-2.69 (m, 6H), 2.66 (s, 3H), 2.56 (s, 3H), 2.44-2.29 (m, 2H). ESI-MS (m/z): 608.4 [M+H]⁺.

### Example 12 Preparation of Compound 12

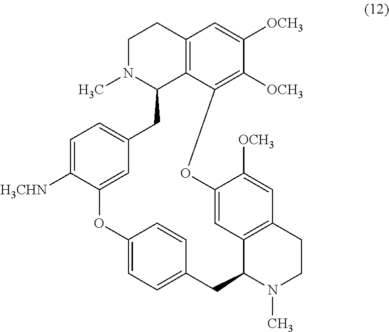

Compound 12 was prepared by acylation with ammonium formate or ethyl formate followed by borane reduction using compound 11 prepared in Example 11.

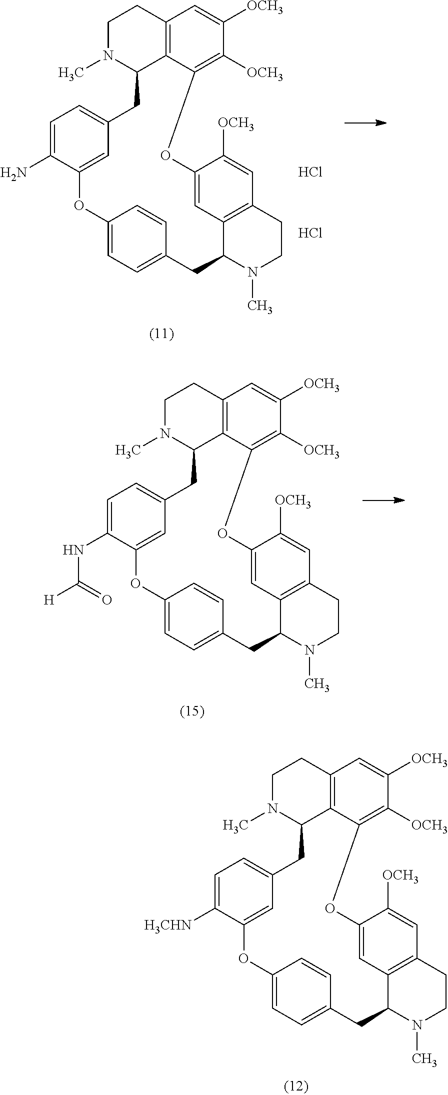

Dihydrochloride of the compound 11 (150 mg, 0.221 mmol) was suspended in ethyl formate (10 mL), triethylamine (0.25 mL, 1.793 mmol, 8.1 eq) was added at room temperature, and the mixture was heated to reflux overnight after the addition, concentrated to dryness to remove the solvent, diluted the residue with DCM(10 mL), washed with 0.5 M dilute hydrochloric acid (2.9 mL), washed with brine, concentrated, and purified by silica gel column chromatography to obtain basic compound 15 (100 mg) as an off-white solid, yield 71%. ESI-MS (m/z): 636.4 [M+H]⁺. Basic Compound 15 (80 mg, 0.126 mmol) was suspended in dry THF(1.5 mL), and BH₃. THF(1 M in THF, 0.63 mL, 5 eq) was added dropwise in an ice bath. After the addition, the mixture was refluxed for 3 h under the protection of nitrogen, cooled to room temperature, cooled in ice bath for 5 min, methanol was slowly added dropwise, excess methanol and concentrated hydrochloric acid (80 μL) were added after no gas was generated, and refluxed for 2 h. The reaction solution was concentrated, and the residue was diluted with DCM, washed with saturated aqueous sodium bicarbonate solution, dried over anhydrous sodium sulfate, concentrated, and purified by silica gel column chromatography to obtain basic compound 12 (48 mg) as an off-white solid, yield 61%. ESI-MS (m/z): 622.5 [M+H]⁺. 45 mg of basic compound 12 was taken into methanol to prepare hydrochloride salt, 15 μL of concentrated hydrochloric acid was added, spin dried, co-boiled with acetonitrile, slurried in MTBE, filtered and dried to obtain 37 mg of dihydrochloride of the compound 12 as a light yellow solid.

### Example 13 Preparation of Compound 13

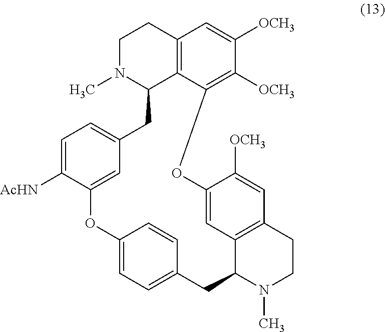 [SMILES: COc1cc2c3cc1Oc1c(OC)c(OC)cc4c1[C@@H](Cc1ccc(NC(C)=O)c(c1)Oc1ccc(cc1)C[C@@H]3N(C)CC2)N(C)CC4]

Compound 13 was prepared by acylation with acetic anhydride using compound 11 prepared in Example 11.

Compound 11 dihydrochloride (75 mg, 0.110 mmol) was dissolved in pyridine (1.5 mL), acetyl chloride (20 L, 0.275 mmol, 2.5 eq) was added dropwise at room temperature, stirred at room temperature for 10 min after addition, and heated to 60° C. for 3 h. The reaction solution was concentrated and purified by silica gel column chromatography to obtain basic compound 13 (65 mg) as an off-white solid, yield 91%.

ESI-MS (m/z): 650.5 [M+H]⁺.

### Example 14 Preparation of Compound 14

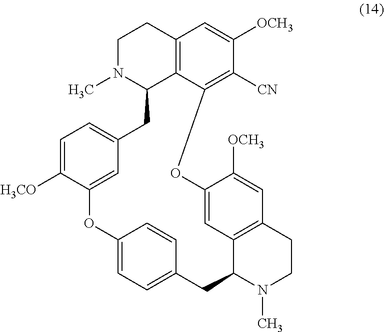 [SMILES: COc1ccc2cc1Oc1ccc(cc1)C[C@H]1c3cc(c(OC)cc3CCN1C)Oc1c(C#N)c(OC)cc3c1[C@@H](C2)N(C)CC3]

Compound 14 was prepared using Fangchinoline as a starting material in a manner similar to that of Example 1.

### Example 15 Preparation of Compound 15

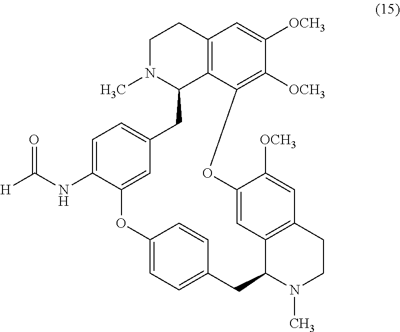 [SMILES: COc1cc2c3cc1Oc1c(OC)c(OC)cc4c1[C@@H](Cc1ccc(NC=O)c(c1)Oc1ccc(cc1)C[C@@H]3N(C)CC2)N(C)CC4]

The preparation method of Compound 15 has been described in Example 12.

### Example 16 Preparation of Compound 16

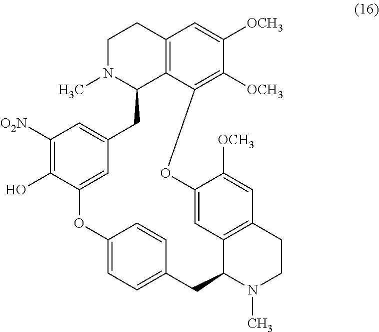 [SMILES: COc1cc2c3cc1Oc1c(OC)c(OC)cc4c1[C@@H](Cc1cc(c(O)c([N+](=O)[O-])c1)Oc1ccc(cc1)C[C@@H]3N(C)CC2)N(C)CC4]

Berbamine dihydrochloride (100 mg, 0.147 mmol) was dissolved in acetic acid (1 mL), 65% nitric acid (13 μL, 1.2 eq) was added dropwise at room temperature, and the reaction solution was stirred at room temperature for 3 min after the addition. The reaction solution was poured into saturated aqueous sodium bicarbonate solution several times in batches, stirred until no gas was generated, DCM was added for extraction, and the organic layer was separated, dried over anhydrous sodium sulfate, concentrated, and purified by silica gel column chromatography to obtain compound 16 (63 mg) as a yellow solid, yield 66%.

¹H NMR (600 MHZ, Chloroform-d) δ 7.67 (d J=8.3 Hz, 1H), 7.55 (d J=2.0 Hz, 1H), 6.97 (td J=8.2, 2.5 Hz, 2H), 6.61 (s, 1H), 6.40 (s, 1H), 6.36 (dd J=8.2, 2.6 Hz, 1H), 6.32 (s, 1H), 5.76 (d J=2.0 Hz, 1H), 4.40 (d J=6.2 Hz, 1H), 3.80 (s, 3H), 3.64 (m, 4H), 3.42 (m, 1H), 3.19 (s, 3H), 3.18-3.06 (m, 3H), 2.93-2.65 (m, 5H), 2.74 (s, 3H), 2.56 (s, 3H), 2.40 (m, 1H), 2.32 (m, 2H). ESI-MS (m/z): 654.4 [M+H]⁺, 652.4 [M−H]⁻.

### Example 17 Preparation of Compound 17

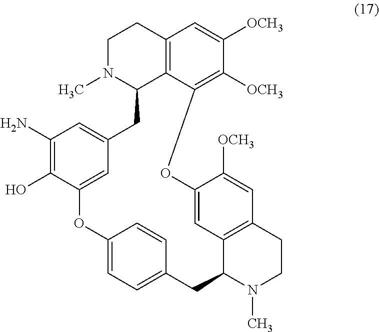 [SMILES: COc1cc2c3cc1Oc1c(OC)c(OC)cc4c1[C@@H](Cc1cc(N)c(O)c(c1)Oc1ccc(cc1)C[C@@H]3N(C)CC2)N(C)CC4]

Compound 16 (260 mg, 0.398 mmol), 10% palladium carbon (50 mg) and ammonium formate (300 mg, 4.762 mmol, 12 eq) were mixed in methanol (10 mL), replaced with nitrogen, and the mixture was heated to reflux, the reaction was carried out for 1 h. TLC showed that raw material was converted completely. The insoluble matter was filtered out, the filter residue was washed with methanol, the filtrate was concentrated, and the residue was diluted with DCM, washed with saturated aqueous sodium bicarbonate solution, washed with brine, dried over anhydrous sodium sulfate, and purified by silica gel column chromatography to obtain basic compound 17 (215 mg) as a light yellow solid, yield 87%. Due to the presence of atropisomerism, the nuclear magnetic spectrum showed two groups of peaks, and the ratio thereof is about 11:9.

Peak of major rotamer ¹H NMR (600 MHZ, Chloroform-d) δ 7.41 (dd J=8.6, 2.3 Hz, 1H), 6.93 (dd J=8.3, 2.5 Hz, 1H), 6.61 (s, 1H), 6.35 (s, 1H), 6.32 (m, 3H), 6.31 (s, 1H), 4.87 (d J=1.8 Hz, 1H), 4.18 (d J=5.5 Hz, 1H), 3.86 (m, 1H), 3.78 (s, 3H), 3.66 (m, 2H), 3.62 (s, 3H), 3.36-3.30 (m, 1H), 3.24-3.07 (m, 2H), 3.18 (s, 3H), 3.06-2.59 (m, 7H), 2.64 (s, 3H), 2.55 (s, 3H), 2.47-2.34 (m, 2H). Peak of minor rotamer ¹H NMR (600 MHZ, Chloroform-d) δ 7.28 (dd J=8.5, 2.3 Hz, 1H), 6.93 (dd J=8.3, 2.5 Hz, 1H), 6.87 (dd J=8.2, 2.7 Hz, 1H), 6.80 (s, 1H), 6.71 (dd J=8.5, 2.7 Hz, 1H), 6.47 (d J=2.0 Hz, 1H), 6.42 (d J=2.2 Hz, 1H), 6.37 (s, 1H), 6.36 (s, 1H), 4.28 (dd J=11.4, 4.2 Hz, 1H), 3.86 (m, 1H), 3.79 (s, 3H), 3.66 (m, 1H), 3.53 (m, 1H), 3.36-3.30 (m, 1H), 3.34 (s, 3H), 3.24-3.07 (m, 2H), 3.14 (s, 3H), 3.06-2.59 (m, 7H), 2.56 (s, 3H), 2.49 (s, 3H), 2.47-2.34 (m, 2H). ESI-MS (m/z): 624.4 [M+H]+, 622.4 [M−H]⁻.

### Example 18 Preparation of Compound 18

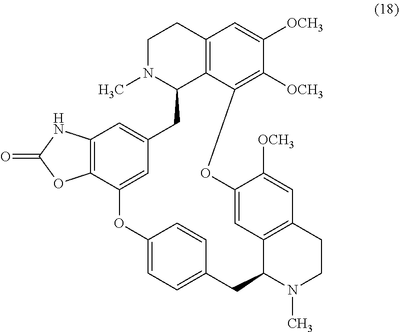 [SMILES: COc1cc2c3cc1Oc1c(OC)c(OC)cc4c1[C@@H](Cc1cc(c5oc(=O)[nH]c5c1)Oc1ccc(cc1)C[C@@H]3N(C)CC2)N(C)CC4]

Dihydrochloride of the Compound 17 (110 mg, 0.158 mmol) was dissolved in DMF(2 mL), N, N′-carbonyldiimidazole (128 mg, 0.790 mmol, 5 eq) was added at room temperature, and stirred at room temperature for 1 h. TLC showed that raw material was converted completely, 10 mL ice water was added, stirred for 5 min, filtered, and the filter cake was washed with water, dried to obtain basic compound 18 (55 mg) as a white solid, yield 54%.

¹H NMR (600 MHZ, Chloroform-d) δ 7.48 (d, J=8.4 Hz, 1H), 6.92 (dd, J=8.3, 2.6 Hz, 1H), 6.87 (dd, J=8.1, 2.2 Hz, 1H), 6.72 (s, 1H), 6.62 (m, 1H), 6.47 (s, 1H), 6.42 (m, 1H), 6.38 (s, 1H), 5.34 (d, J=1.4 Hz, 1H), 4.26 (d, J=5.2 Hz, 1H), 3.80 (s, 3H), 3.70 (m, 1H), 3.65 (s, 3H), 3.37 (m, 1H), 3.27-3.16 (m, 3H), 3.25 (s, 3H), 3.08-2.94 (m, 3H), 2.93-2.84 (m, 2H), 2.74 (m, 1H), 2.69 (s, 3H), 2.67 (s, 3H), 2.56 (m, 1H), 2.45 (m, 1H), ESI-MS (m/z): 650.28 [M+⁺H].

### Example 19 Preparation of Compound 19

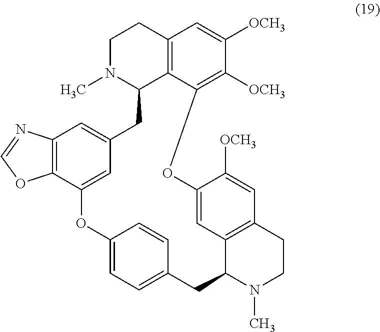 [SMILES: COc1cc2c3cc1Oc1c(OC)c(OC)cc4c1[C@@H](Cc1cc(c5ocnc5c1)Oc1ccc(cc1)C[C@@H]3N(C)CC2)N(C)CC4]

Dihydrochloride of the Compound 17 (105 mg, 0.151 mmol) was dissolved in ethanol (2 mL), trimethyl orthoformate (0.5 mL, 4.53 mmol, 30 eq) and p-toluenesulfonic acid monohydrate (29 mg, 0.153 mmol, 1 eq) were added at room temperature, replaced with nitrogen for three times, and reacted at 65° C. overnight. TLC showed that raw material was converted completely. The mixture was diluted with DCM(10 mL), washed with saturated aqueous sodium bicarbonate solution, dried over anhydrous sodium sulfate, filtered, concentrated, and purified by silica gel column chromatography to obtain basic product compound 19 (75 mg) as an off-white solid, yield 78%.

¹H NMR (600 MHz, Chloroform-d) δ 8.04 (s, 1H), 7.49 (brs, 1H), 7.01 (m, 2H), 6.68 (s, 1H), 6.41 (m, 2H), 6.37 (s, 1H), 6.30 (s, 1H), 5.52 (d, J=1.3 Hz, 1H), 4.23 (d, J=5.7 Hz, 1H), 3.79 (s, 3H), 3.68 (m, 1H), 3.63 (s, 3H), 3.37 (m, 1H), 3.30 (m, 1H), 3.20 (m, 1H), 3.19 (s, 3H), 3.05 (m, 1H), 2.97 (m, 1H), 2.93 (m, 1H), 2.85 (m, 1H), 2.74-2.70 (m, 2H), 2.68 (s, 3H), 2.60 (s, 3H), 2.37 (m, 1H), 2.32-2.27 (m, 2H). ESI-MS (m/z): 634.5 [M+⁺H].

### Example 20 Preparation of Compound 25

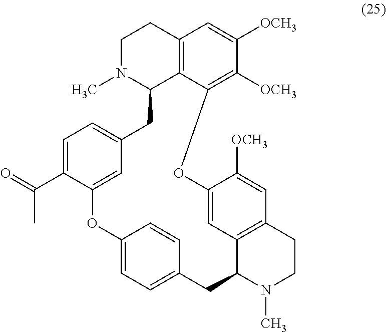 [SMILES: COc1cc2c3cc1Oc1c(OC)c(OC)cc4c1[C@@H](Cc1ccc(C(C)=O)c(c1)Oc1ccc(cc1)C[C@@H]3N(C)CC2)N(C)CC4]

Compound 1 (300 mg, 0.486 mmol) was dissolved in dry tetrahydrofuran (3 mL) under the protection of nitrogen, methyl lithium (0.76 mL, 1.6 M diethyl ether solution, 2.5 eq) was added dropwise in an ice bath, and the reaction solution was kept in ice bath for 1 h after addition. The reaction solution was poured into ice water, 4 N sulfuric acid aqueous solution was added to adjust pH to about 2, and stirred at room temperature for 1 h, the pH was adjusted to weakly basic by sodium bicarbonate, and DCM was added for extraction. The organic phase was dried over anhydrous sodium sulfate, concentrated, and subjected to silica gel column chromatography to obtain basic compound 25 (95 mg) as a light yellow solid, yield 31%.

¹H NMR (600 MHZ, Chloroform-d) δ 7.66 (d, J=8.0 Hz, 1H), 7.55 (m, 1H), 6.97 (m, 2H), 6.93 (m, 1H), 6.63 (s, 1H), 6.38 (s, 1H), 6.33 (s, 1H), 6.29 (dd, J=8.2, 2.6 Hz, 1H), 5.60 (d, J=1.5 Hz, 1H), 4.28 (m, 1H), 3.80 (s, 3H), 3.70 (m, 1H), 3.64 (s, 3H), 3.48 (m, 1H), 3.34-3.23 (m, 2H), 3.19 (s, 3H), 3.07-2.96 (m, 2H), 2.91-2.75 (m, 4H), 2.71 (s, 3H), 2.66 (s, 3H), 2.58 (s, 3H), 2.42-2.34 (m, 3H). ESI-MS (m/z): 635.4 [M+H]⁺, 318.4 [1/2M+H]⁺.

### Example 21 Preparation of Compound 28

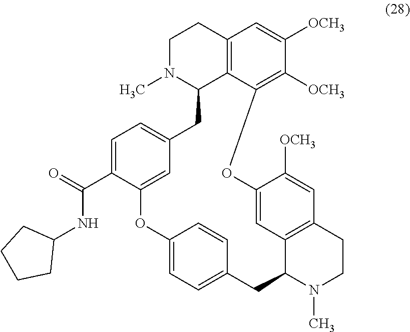 [SMILES: COc1cc2c3cc1Oc1c(OC)c(OC)cc4c1[C@@H](Cc1ccc(C(=O)NC5CCCC5)c(c1)Oc1ccc(cc1)C[C@@H]3N(C)CC2)N(C)CC4]

Compound 7 (80 mg, 0.126 mmol) was dissolved in DCM(1 mL), N, N-dimethylformamide (5 μL, 0.065 mmol, 0.5 eq) was added at room temperature, oxalyl chloride (32 μL, 0.378 mmol, 3 eq) was added dropwise, the mixture was stirred for 2 h under nitrogen protection after the addition, and the mixture was concentrated to remove DCM. The residue was mixed with pyridine (1.5 mL), cyclopentylamine (62 μL, 0.63 mmol, 5 eq) was added, stirred at 60° C. for 4 h, concentrated, and purified by silica gel column chromatography to obtain basic compound 28 (40 mg) as a pale yellow solid, yield 45%.

¹H NMR (600 MHz, Chloroform-d) δ 8.04 (d, J=8.0 Hz, 1H), 7.77 (m, 1H), 7.03 (dd, J=8.3, 2.4 Hz, 1H), 6.97 (dd, J=8.0, 2.2 Hz, 1H), 6.62 (s, 1H), 6.45 (m, 1H), 6.41 (m, 1H), 6.33 (s, 1H), 6.29 (dd, J=8.3, 2.5 Hz, 1H), 5.53 (s, 1H), 4.50 (m, 1H), 4.40 (h, J=6.7 Hz, 1H), 3.95-3.72 (m, 2H), 3.81 (s, 3H), 3.64 (s, 3H), 3.55 (m, 1H), 3.32 (m, 1H), 3.20 (s, 3H), 3.16-3.05 (m, 2H), 3.03-2.67 (m, 5H), 2.80 (s, 3H), 2.62 (s, 3H), 2.50-2.30 (m, 2H), 2.00 (dd, J=12.6, 6.5 Hz, 2H), 1.59 (m, 4H), 1.46 (dd, J=12.4, 6.2 Hz, 2H). ESI-MS (m/z): 704.5 [M+H]⁺.

### Example 22 Preparation of Compound 33

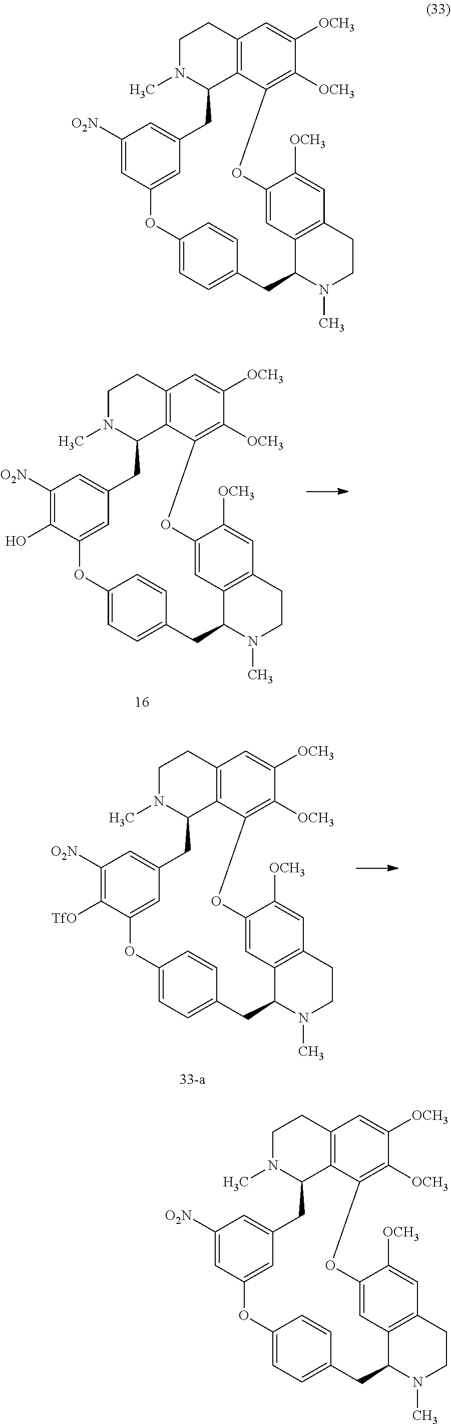 [SMILES: COc1cc2c3cc1Oc1c(OC)c(OC)cc4c1[C@@H](Cc1cc(cc([N+](=O)[O-])c1)Oc1ccc(cc1)C[C@@H]3N(C)CC2)N(C)CC4 ; COc1cc2c3cc1Oc1c(OC)c(OC)cc4c1[C@@H](Cc1cc(c(O)c([N+](=O)[O-])c1)Oc1ccc(cc1)C[C@@H]3N(C)CC2)N(C)CC4 ; COc1cc2c3cc1Oc1c(OC)c(OC)cc4c1[C@@H](Cc1cc(c(OS(=O)(=O)C(F)(F)F)c([N+](=O)[O-])c1)Oc1ccc(cc1)C[C@@H]3N(C)CC2)N(C)CC4 ; COc1cc2c3cc1Oc1c(OC)c(OC)cc4c1[C@@H](Cc1cc(cc([N+](=O)[O-])c1)Oc1ccc(cc1)C[C@@H]3N(C)CC2)N(C)CC4]

Compound 16 (470 mg, 0.719 mmol) was dissolved in DCM(6 mL), pyridine (0.29 mL, 3.604 mmol, 5 eq) and DMAP(8.8 mg, 0.072 mmol, 0.1 eq) were added, the mixture was kept in ice bath for 5 min, trifluoromethanesulfonic anhydride (0.24 mL, 1.438 mmol, 2 eq) was added dropwise, and the temperature was maintained for 10 min after the addition. TLC showed that raw material was converted completely, diluted with DCM, washed with 0.5 M dilute hydrochloric acid, dried over anhydrous sodium sulfate, and purified by silica gel column chromatography to obtain product 33-a (480 mg) as a yellow solid, yield 85%, ESI-MS (m/z): 786.30 [M+H]⁺.

33-a (100 mg, 0.127 mmol), palladium acetate (3 mg, 0.013 mmol, 0.1 eq), 2-dicyclohexylphosphon-2,4,6-triisopropylbiphenyl (13 mg, 0.027 mmol, 0.21 eq), formic acid (16 μL, 0.424 mmol, 3.3 eq) and N, N-diisopropylethylamine (74 μL, 0.424 mmol, 3.3 eq) were mixed in dried dioxane (1.5 mL), replaced with nitrogen for many times, and stirred at 90° C. for 30 min. The reaction solution was cooled to room temperature, EA was added to dilute, insoluble substances were filtered out, the filter residue was washed with EA, and the filtrate was diluted with EA, washed with water, dried over anhydrous sodium sulfate, and purified by silica gel column chromatography to obtain compound 33 (44 mg) as a pale yellow solid, yield 54%.

¹H NMR (600 MHz, Chloroform-d) δ 7.71 (s, 1H), 7.68 (s, 1H), 7.59 (brs, 1H), 6.97 (m, 2H), 6.62 (s, 1H), 6.38 (s, 1H), 6.30 (s, 1H), 6.28 (dd, J=8.1, 2.6 Hz, 1H), 5.79 (s, 1H), 4.30 (m, 1H), 3.80 (s, 3H), 3.68 (m, 1H), 3.63 (s, 3H), 3.50 (m, 1H), 3.30 (m, 1H), 3.26 (m, 1H), 3.19 (s, 3H), 3.12-3.00 (m, 2H), 2.92-2.73 (m, 4H), 2.71 (s, 3H), 2.59 (s, 3H), 2.38 (m, 1H), 2.31-2.25 (m, 2H). ESI-MS (m/z): 638.21 [M+H]⁺.

### Example 23 Preparation of Compound 40

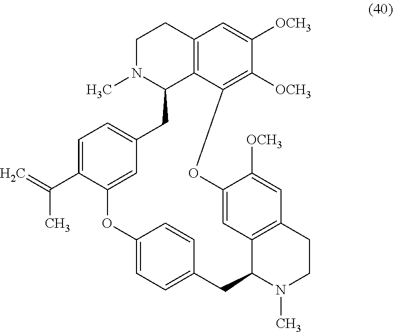 [SMILES: C=C(C)c1ccc2cc1Oc1ccc(cc1)C[C@H]1c3cc(c(OC)cc3CCN1C)Oc1c(OC)c(OC)cc3c1[C@@H](C2)N(C)CC3]

Compound 1-a(50 mg, 0.068 mmol), palladium acetate (1.6 mg, 0.007 mmol, 0.1 eq), 2-dicyclohexylphospho-2,4, 6-triisopropylbiphenyl (6.5 mg, 0.014 mmol, 0.2 eq), and isopropenyl boronic acid pinacol ester (25 μL, 0.135 mmol, 2 eq) were mixed in dioxane (1 mL) and water (0.2 mL), sodium carbonate (22 mg, 0.204 mmol, 3 eq) was added, replaced with nitrogen for three times, gradually heated to 75° C., and reacted overnight. The reaction solution was cooled to room temperature, diluted with DCM, washed with water, the organic phase was separated, dried over anhydrous sodium sulfate, concentrated, and purified by silica gel column chromatography to give basic compound 40 (21 mg) as a pale yellow solid, yield 47%. Due to the presence of atropisomerism, the nuclear magnetic spectrum showed two groups of peaks, and the ratio thereof is about 5.3:4.7.

Peak of major rotamer ¹H NMR (600 MHZ, Chloroform-d) δ 7.34 (d, J=7.9 Hz, 1H), 7.30-6.98 (m, 1H), 6.96 (d, J=1.6 Hz, 1H), 6.90 (dd, J=8.2, 2.5 Hz, 1H), 6.78 (s, 1H), 6.59 (dd, J=8.4, 2.7 Hz, 1H), 6.45 (s, 1H), 6.43 (s, 1H), 6.39 (m, 1H), 5.22 (dd, J=2.1, 1.1 Hz, 1H), 5.19 (m, 1H), 4.68 (d, J=11.6 Hz, 1H), 3.92 (m, 1H), 3.82 (s, 3H), 3.71 (m, 1H), 3.49 (m, 1H), 3.41 (s, 3H), 3.25-3.05 (m, 3H), 3.17 (s, 3H), 2.99-2.71 (m, 4H), 2.78 (s, 3H), 2.66 (s, 3H), 2.62-2.42 (m, 2H), 2.18 (s, 3H). Peak of minor rotamer ¹H NMR (600 MHZ, Chloroform-d) δ 7.67 (s, 1H), 7.34 (d, J=7.9 Hz, 1H), 7.11 (d, J=7.6 Hz, 1H), 6.93 (dd, J=8.3, 2.6 Hz, 1H), 6.90 (dd, J=8.2, 2.5 Hz, 1H), 6.67 (s, 1H), 6.41 (s, 1H), 6.36 (s, 1H), 6.29 (dd, J=8.2 Hz, 2.6 Hz, 1H), 5.41 (s, 1H), 5.19 (m, 1H), 5.15 (m, 1H), 4.48 (m, 1H), 3.81 (s, 3H), 3.71 (m, 1H), 3.64 (s, 3H), 3.49 (m, 1H), 3.25-3.05 (m, 3H), 3.21 (s, 3H), 2.99-2.71 (m, 5H), 2.74 (s, 3H), 2.66 (s, 3H), 2.62-2.42 (m, 1H), 2.15 (s, 3H). ESI-MS (m/z): 633.6 [M+H]⁺.

### Example 24 Preparation of Compound 41

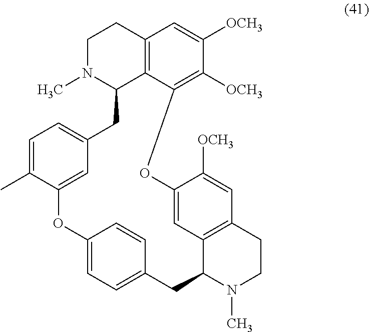 [SMILES: COc1cc2c3cc1Oc1c(OC)c(OC)cc4c1[C@@H](Cc1ccc(C)c(c1)Oc1ccc(cc1)C[C@@H]3N(C)CC2)N(C)CC4]

Compound 1-a (200 mg, 0.270 mmol), palladium acetate (6 mg, 0.027 mmol, 0.1 eq), 2-dicyclohexylphosphon-2,4,6-triisopropylbiphenyl (26 mg, 0.054 mmol, 0.2 eq), and methylboronic acid (32 mg, 0.540 mmol, 2 eq) were mixed in dioxane (4 mL) and water (0.8 mL), potassium carbonate (112 mg, 0.810 mmol, 3 eq) was added, and the mixture was replaced with nitrogen for three times. The mixture was gradually raised to 90° C. and kept for 8 hours. TLC showed that the raw materials were reacted completely. After the reaction solution was cooled to room temperature, diatomite was used for aid filtration, and the filter residue was washed with ethyl acetate. The filtrate was diluted with ethyl acetate, washed with saturated brine, dried over anhydrous sodium sulfate, concentrated, and purified by silica gel column chromatography to obtain basic compound 41 (76 mg) as a light yellow solid, yield 46%. Due to the presence of atropisomerism, the nuclear magnetic spectrum showed two groups of peaks, and the ratio thereof is about 3:2.

Peak of major rotamer ¹H NMR (600 MHz, Chloroform-d) δ 7.48 (d, J=8.2 Hz, 1H), 6.98 (m, 1H), 6.92 (m, 1H), 6.88 (dd, J=8.2, 2.7 Hz, 1H), 6.81 (m, 1H), 6.66 (s, 1H), 6.37 (s, 1H), 6.33 (s, 1H), 6.29 (dd, J=8.1, 2.5 Hz, 1H), 5.41 (d, J=1.5 Hz, 1H), 4.27 (d, J=5.7 Hz, 1H), 3.79 (s, 3H), 3.69 (m, 1H), 3.63 (s, 3H), 3.43 (m, 1H), 3.34-3.22 (m, 2H), 3.20 (s, 3H), 3.10-2.89 (m, 3H), 2.86-2.64 (m, 4H), 2.69 (s, 3H), 2.60 (m, 1H), 2.59 (s, 3H), 2.42 (m, 1H), 2.27 (s, 3H). Peak of minor rotamer ¹H NMR (600 MHZ, chloroform-d) δ 7.29 (dd, J=8.5, 2.3 Hz, 1H), 7.24 (d, J=7.8 Hz, 1H), 7.07 (m, 1H), 6.98 (m, 1H), 6.92 (m, 1H), 6.75 (s, 1H), 6.54 (dd, J=8.4, 2.7 Hz, 1H), 6.41 (s, 1H), 6.40 (s, 1H), 6.38 (m, 1H), 4.42 (m, 1H), 3.81 (s, 3H), 3.69 (m, 1H), 3.43 (m, 1H), 3.38 (s, 3H), 3.34-3.22 (m, 1H), 3.18 (s, 3H), 3.10-2.89 (m, 4H), 2.86-2.64 (m, 4H), 2.61 (s, 3H), 2.60 (m, 1H), 2.43 (s, 3H), 2.42 (m, 1H), 2.37 (s, 3H). ESI-MS (m/z): 607.4 [M+H]⁺.

### Example 25 Preparation of Compound 42

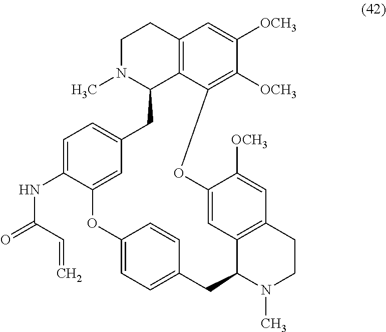 [SMILES: C=CC(=O)Nc1ccc2cc1Oc1ccc(cc1)C[C@H]1c3cc(c(OC)cc3CCN1C)Oc1c(OC)c(OC)cc3c1[C@@H](C2)N(C)CC3]

Compound 11 (50 mg, 0.082 mmol) was dissolved in DCM(2.5 mL), and pyridine (33 μL, 0.412 mmol, 5 eq) and acryloyl chloride (13 μL, 0.165 mmol, 2 eq) were added in sequence. After the addition, the reaction was conducted for 20 min at room temperature. TLC showed that raw materials were converted completely, and the reaction solution was spin-dried, and purified by silica gel column chromatography to obtain basic compound 42 (32 mg) as an off-white solid, yield 58.7%.

¹H NMR (600 MHZ, Chloroform-d) δ 8.31 (d, J=8.2 Hz, 1H), 7.88 (m, 1H), 6.97 (d, J=8.3 Hz, 2H), 6.65 (s, 1H), 6.46-6.41 (m, 2H), 6.40 (s, 1H), 6.33 (s, 1H), 6.32-6.26 (m, 2H), 5.74 (dd, J=10.3, 1.4 Hz, 1H), 5.42 (m, 1H), 4.40 (m, 1H), 3.81 (s, 3H), 3.68 (m, 1H), 3.64 (s, 3H), 3.42 (m, 1H), 3.20 (m, 4H), 3.14-2.81 (m, 6H), 2.80-2.57 (m, 2H), 2.75 (s, 3H), 2.62 (s, 3H), 2.53-2.36 (m, 2H). ESI-MS (m/z): 662.5 [M+H]⁺, 331.9 [1/2M+H]⁺.

### Example 26 Preparation of Compound 43

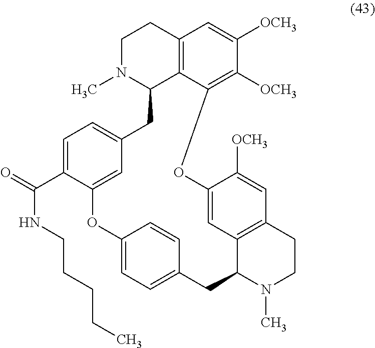 [SMILES: CCCCCNC(=O)c1ccc2cc1Oc1ccc(cc1)C[C@H]1c3cc(c(OC)cc3CCN1C)Oc1c(OC)c(OC)cc3c1[C@@H](C2)N(C)CC3]

Compound 7 (20 mg, 0.031 mmol) was dissolved in DCM(1 mL), N, N-dimethylformamide (1.2 μL, 0.016 mmol, 0.5 eq) was added at room temperature, oxalyl chloride (8 μL, 0.095 mmol, 3 eq) was added dropwise, the mixture was stirred for 2 h under nitrogen protection after the addition, and the mixture was concentrated to remove DCM. The residue was mixed with pyridine (1 mL), n-pentylamine (16 μL, 0.158 mmol, 5 eq) was added at 60° C., stirred for 4 h, concentrated, and purified by silica gel column chromatography to obtain basic compound 43 (12 mg) as a pale yellow solid, yield 54%.

ESI-MS (m/z): 706.5 [M+H]+, 1411.2 [2M+H]⁺. ¹H NMR (600 MHZ, Chloroform-d) δ 8.05 (d, J=8.0 Hz, 1H), 7.79 (m, 1H), 7.03-6.94 (m, 3H), 6.62 (s, 1H), 6.40 (s, 1H), 6.32 (s, 1H), 6.29 (m, 1H), 5.56 (m, 1H), 4.42 (m, 1H), 3.81 (s, 3H), 3.74 (m, 1H), 3.64 (s, 3H), 3.47-3.22 (m, 5H), 3.20 (s, 3H), 3.09 (m, 2H), 2.92 (m, 2H), 2.87-2.69 (m, 5H), 2.60 (s, 3H), 2.53-2.25 (m, 3H), 1.57 (m, 2H), 1.30 (m, 2H), 1.25 (m, 2H), 0.83 (m, 3H). ESI-MS (m/z): 706.5 [M+H]⁺, 1411.2 [2M+H]⁺.

### Example 27 Preparation of Compound 44

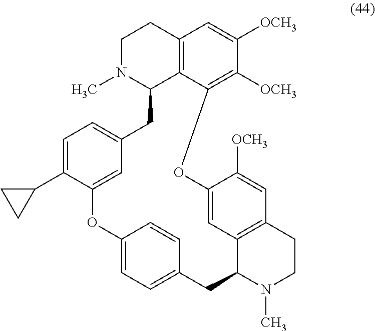 [SMILES: COc1cc2c3cc1Oc1c(OC)c(OC)cc4c1[C@@H](Cc1ccc(C5CC5)c(c1)Oc1ccc(cc1)C[C@@H]3N(C)CC2)N(C)CC4]

Compound 1-a (150 mg, 0.203 mmol), palladium acetate (4.5 mg, 0.02 mmol, 0.1 eq), 2-dicyclohexylphospho-2,4, 6-triisopropylbiphenyl (19.5 mg, 0.041 mmol, 0.2 eq), and cyclopropyl boronic acid (35 mg, 0.406 mmol, 2 eq) were mixed in dioxane (4 mL) and water (0.8 mL), potassium carbonate (84 mg, 0.608 mmol, 3 eq) was added, replaced with nitrogen for three times, the mixture was gradually raised to 90° C., and the reaction was carried out overnight. After the reaction solution was cooled to room temperature, diatomite was used for aid filtration. The filter residue was washed with ethyl acetate. The filtrate was diluted with ethyl acetate, washed with saturated brine, dried over anhydrous sodium sulfate, concentrated, and purified by silica gel column chromatography to give basic compound 44 (67 mg) as a pale yellow solid, yield 52%.

ESI-MS (m/z): 633.4 [M+H]⁺.

### Example 28 Preparation of Compound 45

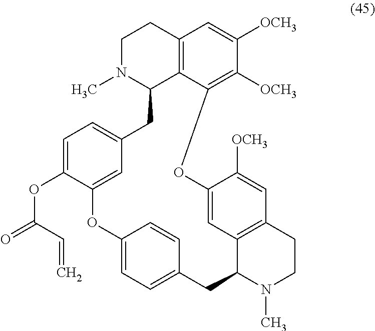 [SMILES: C=CC(=O)Oc1ccc2cc1Oc1ccc(cc1)C[C@H]1c3cc(c(OC)cc3CCN1C)Oc1c(OC)c(OC)cc3c1[C@@H](C2)N(C)CC3]

Berbamine(150 mg, 0.220 mmol) was suspended in DCM(2 mL), triethylamine (0.154 mL, 1.101 mmol, 5 eq) was added, stirred in ice bath for 5 min, acryloyl chloride (27 μL, 0.33 mmol, 1.5 eq) was added dropwise, stirred at room temperature for 30 min after the addition, diluted with DCM, washed with saturated aqueous ammonium chloride solution, dried over anhydrous sodium sulfate, concentrated, and purified by silica gel column chromatography to obtain compound 45 (64 mg) as an off-white solid, yield 45%.

ESI-MS (m/z): 663.22 [M+H]⁺.

### Example 29 Preparation of Compound 46

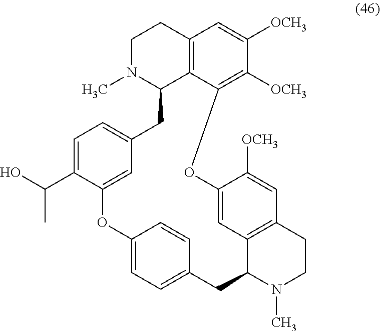 [SMILES: COc1cc2c3cc1Oc1c(OC)c(OC)cc4c1[C@@H](Cc1ccc(C(C)O)c(c1)Oc1ccc(cc1)C[C@@H]3N(C)CC2)N(C)CC4]

Compound 25 (100 mg, 0.161 mmol) was mixed with methyl magnesium bromide (2.5 mL, 3.0 M 2-methyl tetrahydrofuran solution) in ice bath under nitrogen protection, slowly warmed to room temperature, and stirred for 2 h. The reaction solution was carefully poured into ice water, dichloromethane and ammonium chloride were added, the organic layer was separated, washed with brine, dried over anhydrous sodium sulfate, concentrated, and purified by silica gel column chromatography to obtain basic compound 46 (50 mg) as a light yellow solid, yield 49%.

### Example 30 Preparation of Compound 47

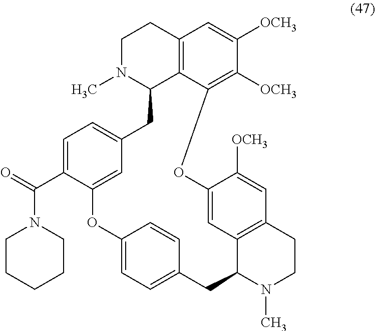 [SMILES: COc1cc2c3cc1Oc1c(OC)c(OC)cc4c1[C@@H](Cc1ccc(C(=O)N5CCCCC5)c(c1)Oc1ccc(cc1)C[C@@H]3N(C)CC2)N(C)CC4]

Compound 7 (150 mg, 0.236 mmol) was dissolved in DCM(2 mL), DMF(1.8 μL, 0.024 mmol) was added, oxalyl chloride (0.1 mL, 1.18 mmol) was added dropwise at room temperature, and the reaction solution was spin-dried after 30 min under the protection of nitrogen after the addition, and dissolved in DCM(2 mL). The solution was added dropwise to a solution of piperidine (108 μL, 1.18 mmol) in DCM(2 mL), stirred for 30 min after the addition, saturated aqueous ammonium chloride solution was added to wash, and the organic phase was dried over anhydrous sodium sulfate, concentrated, and purified by silica gel column chromatography to obtain compound 47 (115 mg, 0.163 mmol) as a light yellow solid, yield 69%.

¹H NMR (600 MHZ, Chloroform-d) δ 7.58 (m, 1H), 7.15 (d, J=7.7 Hz, 1H), 7.01 (m, 1H), 6.96-6.81 (m, 2H), 6.63 (s, 1H), 6.39 (s, 1H), 6.37 (m, 1H), 6.32 (s, 1H), 5.41 (m, 1H), 4.33 (m, 1H), 3.81 (s, 3H), 3.80-3.66 (m, 4H), 3.64 (s, 3H), 3.49-3.26 (m, 3H), 3.19 (s, 3H), 3.14-2.67 (m, 7H), 2.74 (s, 3H), 2.63 (s, 3H), 2.55-2.28 (m, 3H), 1.68-1.57 (m, 6H). ESI-MS (m/z): 704.7 [M+H]⁺, 353.1 [1/2M+H]⁺.

### Example 31 Preparation of Compound 27

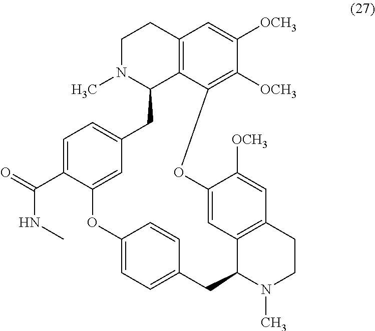 [SMILES: CNC(=O)c1ccc2cc1Oc1ccc(cc1)C[C@H]1c3cc(c(OC)cc3CCN1C)Oc1c(OC)c(OC)cc3c1[C@@H](C2)N(C)CC3]

Compound 7 (65 mg, 0.102 mmol) was dissolved in DCM(1 mL), DMF(0.8 μL, 0.01 mmol) was added, oxalyl chloride (44 μL, 0.51 mmol) was added dropwise at room temperature, and the reaction solution was spin-dried after 30 min under the protection of nitrogen after the addition, and dissolved in DCM(1 mL). The solution was added dropwise to a solution of 28% methylamine aqueous solution (115 mg, 1.02 mmol) in THF(2 mL) in an ice bath. After the addition, the mixture was stirred for 10 min, diluted with DCM, washed with saturated ammonium chloride aqueous solution, and the organic phase was dried over anhydrous sodium sulfate, concentrated, and purified by silica gel column chromatography to give compound 27 (45 mg, 0.069 mmol) as a pale yellow solid, yield 68%.

¹H NMR (600 MHz, Chloroform-d) δ 8.05 (d, J=8.0 Hz, 1H), 7.81 (q, J=5.0 Hz, 1H), 7.51 (m, 1H), 6.98 (m, 3H), 6.61 (s, 1H), 6.36 (s, 1H), 6.30 (m, 2H), 5.58 (d, J=1.6 Hz, 1H), 4.20 (d, J=5.8 Hz, 1H), 3.78 (s, 3H), 3.68 (t, J=3.5 Hz, 1H), 3.63 (s, 3H), 3.43-3.37 (m, 1H), 3.36-3.20 (m, 2H), 3.18 (s, 3H), 3.05 (m, 1H), 2.97 (d, J=4.8 Hz, 3H), 2.95 (m, 1H), 2.88-2.82 (m, 2H), 2.72 (m, 2H), 2.68 (s, 3H), 2.57 (s, 3H), 2.41-2.27 (m, 3H). ESI-MS (m/z): 650.29 [M+H]⁺.

### Example 32 Preparation of Compound 49

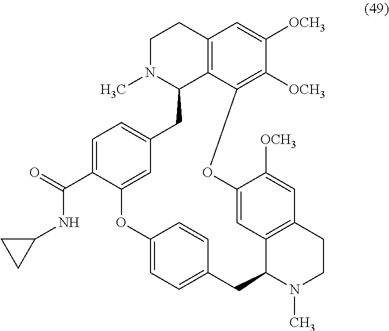 [SMILES: COc1cc2c3cc1Oc1c(OC)c(OC)cc4c1[C@@H](Cc1ccc(C(=O)NC5CC5)c(c1)Oc1ccc(cc1)C[C@@H]3N(C)CC2)N(C)CC4]

Compound 7 (150 mg, 0.236 mmol) was dissolved in DCM(2 mL), DMF(1.8 μL, 0.024 mmol) was added, oxalyl chloride (0.1 mL, 1.18 mmol) was added dropwise at room temperature, and the reaction solution was spin-dried after 30 min under the protection of nitrogen after the addition, and dissolved in DCM(2 mL). The solution was added dropwise to a solution of cyclopropylamine (82 μL, 1.18 mmol) in DCM(2 mL). After the addition, the mixture was stirred for 10 min, washed with saturated aqueous ammonium chloride solution, and the organic phase was dried over anhydrous sodium sulfate, concentrated, and purified by silica gel column chromatography to obtain compound 49 (105 mg, 0.156 mmol) as a light yellow solid, yield 66%.

¹H NMR (600 MHz, Chloroform-d) δ 8.05 (d, J=8.0 Hz, 1H), 7.85 (m, 1H), 7.64 (m, 1H), 6.99 (m, 3H), 6.61 (s, 1H), 6.38 (s, 1H), 6.31 (s, 1H), 6.28 (dd, J=8.2, 2.6 Hz, 1H), 5.55 (d, J=1.4 Hz, 1H), 4.36 (m, 1H), 3.80 (s, 3H), 3.71 (m, 1H), 3.63 (s, 3H), 3.55 (m, 1H), 3.38-3.23 (m, 2H), 3.18 (s, 3H), 3.11-3.03 (m, 2H), 2.90 (m, 2H), 2.82 (m, 2H), 2.73 (s, 3H), 2.66 (m, 1H), 2.58 (s, 3H), 2.43-2.30 (m, 3H), 0.79 (m, 2H), 0.53 (m, 2H). ESI-MS (m/z): 676.6 [M+H]⁺, 339.0 [1/2M+H]⁺.

### Example 33 Preparation of Compound 50

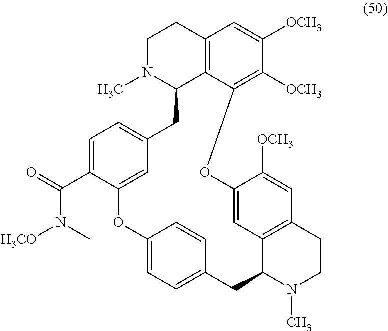 [SMILES: COc1cc2c3cc1Oc1c(OC)c(OC)cc4c1[C@@H](Cc1ccc(C(=O)N(C)OC)c(c1)Oc1ccc(cc1)C[C@@H]3N(C)CC2)N(C)CC4]

Compound 7 (550 mg, 0.865 mmol) was dissolved in DCM(10 mL), DMF(6.6 μL, 0.087 mmol) was added, oxalyl chloride (0.37 mL, 4.324 mmol) was added dropwise at room temperature, and the reaction solution was spin-dried after 30 min under the protection of nitrogen after the addition, and dissolved in DCM(10 mL). The solution was added dropwise to a solution of dimethylhydroxylamine hydrochloride (420 mg, 4.325 mmol) and triethylamine (0.60 mL, 4.325 mmol) in DCM(20 mL) at room temperature for 20 min. After addition, the mixture was stirred for 10 min, and washed with saturated aqueous ammonium chloride solution. The organic phase was dried over anhydrous sodium sulfate, concentrated, and purified by silica gel column chromatography to obtain compound 50 (410 mg, 0.604 mmol) as a light yellow solid, yield 70%.

¹H NMR (600 MHZ, Chloroform-d) δ 7.54 (m, 1H), 7.18 (d, J=7.6 Hz, 1H), 6.96-6.87 (m, 3H), 6.62 (s, 1H), 6.38 (s, 1H), 6.32-6.27 (m, 2H), 5.49 (s, 1H), 4.31 (m, 1H), 3.79 (s, 3H), 3.77 (m, 1H), 3.63 (s, 3H), 3.45-3.22 (m, 6H), 3.18 (s, 3H), 3.10-2.62 (m, 12H), 2.60 (s, 3H), 2.54-2.24 (m, 3H). ESI-MS (m/z): 680.27 [M+H]⁺, 340.80 [1/2M+H]⁺. Example 34 Preparation of Compound 51

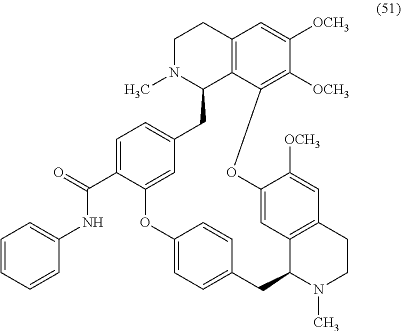 [SMILES: COc1cc2c3cc1Oc1c(OC)c(OC)cc4c1[C@@H](Cc1ccc(C(=O)Nc5ccccc5)c(c1)Oc1ccc(cc1)C[C@@H]3N(C)CC2)N(C)CC4]

Compound 7 (150 mg, 0.236 mmol) was dissolved in DCM(2 mL), DMF(1.8 μL, 0.024 mmol) was added, oxalyl chloride (0.1 mL, 1.18 mmol) was added dropwise at room temperature, and the reaction solution was spin-dried after 30 min under the protection of nitrogen after the addition, and dissolved in DCM(2 mL). The solution was added dropwise to a solution of aniline (0.11 mL, 1.18 mmol) and pyridine (0.09 mL, 1.18 mmol) in DCM(3 mL) at room temperature. After the addition, the mixture was stirred for 10 min, washed with saturated aqueous ammonium chloride solution, and the organic phase was dried over anhydrous sodium sulfate, concentrated, and purified by silica gel column chromatography to obtain compound 51 (100 mg, 0.14 mmol) as a light yellow solid, yield 60%.

¹H NMR (600 MHZ, Chloroform-d) δ 9.68 (m, 1H), 8.16 (d, J=8.0 Hz, 1H), 8.02 (m, 1H), 7.60 (dd, J=13.5, 8.0 Hz, 2H), 7.31 (t, J=7.8 Hz, 2H), 7.09 (m, 2H), 6.99 (m, 1H), 6.67 (s, 1H), 6.45 (s, 1H), 6.38 (m, 2H), 5.56 (s, 1H), 4.65 (m, 1H), 4.00 (m, 2H), 3.83 (s, 3H), 3.66 (s, 3H), 3.45 (m, 2H), 3.27 (m, 1H), 3.22 (s, 3H), 3.14 (m, 2H), 3.06-2.94 (m, 2H), 2.93-2.76 (m, 5H), 2.70 (s, 3H), 2.58-2.30 (m, 2H). ESI-MS (m/z): 712.23 [M+H]⁺, 356.83 [1/2M+H]⁺.

### Example 35 Preparation of Compound 52

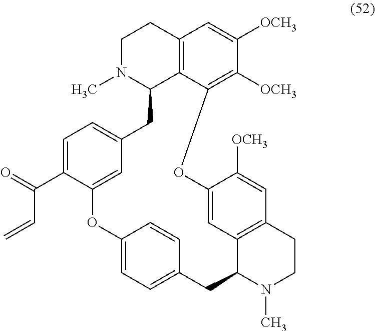 [SMILES: C=CC(=O)c1ccc2cc1Oc1ccc(cc1)C[C@H]1c3cc(c(OC)cc3CCN1C)Oc1c(OC)c(OC)cc3c1[C@@H](C2)N(C)CC3]

Compound 50 (130 mg, 0.191 mmol) was dissolved in THF(2 mL), vinyl magnesium bromide (0.58 mL, 0.58 mmol, 3 eq, 1 M in THF) was added dropwise in an ice bath under the protection of nitrogen, and the reaction mixture was reacted at room temperature for 1 h. The reaction solution was carefully added to 1 N hydrochloric acid (2 mL), and stirred at room temperature for 30 min, sodium bicarbonate was added to adjust pH to weak alkaline, DCM was added to extract, and the organic phase was dried over anhydrous sodium sulfate, concentrated, and purified by silica gel column chromatography to obtain compound 52 (90 mg, 0.139 mmol) as a light yellow solid, yield 73%.

¹H NMR (600 MHz, Chloroform-d) δ 7.56 (d, J=7.9 Hz, 1H), 7.17 (td, J=17.5, 10.4 Hz, 1H), 6.96 (m, 3H), 6.64 (s, 1H), 6.44 (d, J=14.9 Hz, 1H), 6.40 (s, 1H), 6.36-6.31 (m, 2H), 6.28 (m, 1H), 5.73 (dd, J=10.4, 1.9 Hz, 1H), 5.55 (s, 1H), 4.45 (m, 1H), 3.81 (s, 3H), 3.76 (m, 1H), 3.64 (s, 3H), 3.52 (m, 1H), 3.42 (m, 1H), 3.33 (m, 1H), 3.20 (s, 3H), 3.17-3.01 (m, 2H), 2.97-2.68 (m, 7H), 2.61 (s, 3H), 2.48-2.28 (m, 3H). ESI-MS (m/z): 647.5 [M+H]*, 324.5 [1/2M+H]⁺, 1292.9 [2M+H]⁺.

### Example 36 Preparation of Compound 53

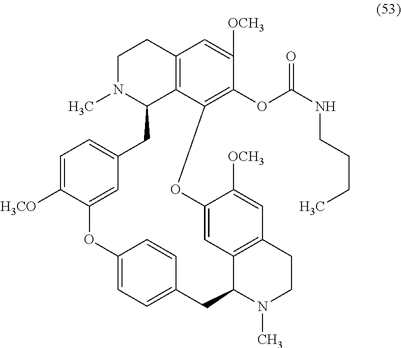 [SMILES: CCCCNC(=O)Oc1c(OC)cc2c3c1Oc1cc4c(cc1OC)CCN(C)[C@H]4Cc1ccc(cc1)Oc1cc(ccc1OC)C[C@H]3N(C)CC2]

Fangchinoline (100 mg, 0.164 mmol, 1 eq) was dissolved in DCM(1 mL), triethylamine (91 μL, 0.653 mmol, 4 eq) was added, cooled to 0˜5° C. in an ice bath, n-butyl isocyanate (28 μL, 0.246 mmol, 1.5 eq) was slowly added dropwise, and the reaction was carried out at room temperature for 12 h after the addition. TLC showed that the raw materials was reacted completely. The reaction solution was diluted with DCM and transferred to a separatory funnel, an appropriate amount of ammonium chloride solution was added, mixed evenly, stood and layered, and the organic phase was separated, washed with saturated brine once, and dried over anhydrous Na₂SO₄, filtered, and the filtrate was spin-dried and subjected to silica gel column chromatography (DCM:MeOH=70:1-60:1) to give compound 53 (85 mg) as an off-white solid, yield 73%.

ESI-MS (m/z): 708.22 [M+H]⁺, 354.82 [1/2M+H]⁺.

### Example 37 Preparation of Compound 54

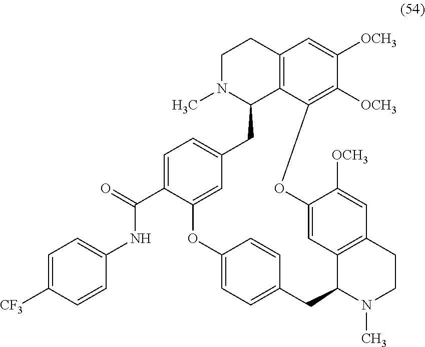 [SMILES: COc1cc2c3cc1Oc1c(OC)c(OC)cc4c1[C@@H](Cc1ccc(C(=O)Nc5ccc(C(F)(F)F)cc5)c(c1)Oc1ccc(cc1)C[C@@H]3N(C)CC2)N(C)CC4]

Compound 7 (50 mg, 0.079 mmol) was dissolved in DCM(1 mL), oxalyl chloride (33 μL, 0.393 mmol) was added dropwise at room temperature, and the reaction solution was spin-dried after 30 min under the protection of nitrogen after the addition, and dissolved in DCM(1 mL). The solution was dropped into a solution of 4-trifluoromethylaniline (49 μL, 0.393 mmol) and pyridine (32 μL, 0.393 mmol) in DCM(2 mL) at room temperature. After addition, the mixture was stirred for 10 min, washed with saturated aqueous ammonium chloride solution, and the organic phase was dried over anhydrous sodium sulfate, concentrated, and purified by silica gel column chromatography to give compound 54 (32 mg, 0.14 mmol) as a pale yellow solid, yield 52%.

ESI-MS (m/z): 780.5 [M+H]⁺, 778.5 [M−H]⁻.

### Example Preparation of Compound 55

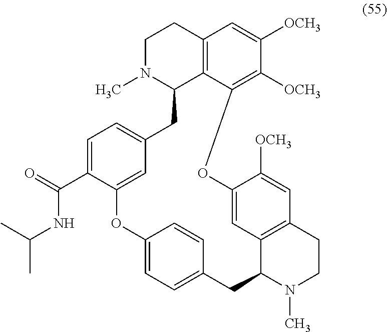 [SMILES: COc1cc2c3cc1Oc1c(OC)c(OC)cc4c1[C@@H](Cc1ccc(C(=O)NC(C)C)c(c1)Oc1ccc(cc1)C[C@@H]3N(C)CC2)N(C)CC4]

Compound 7 (50 mg, 0.079 mmol) was dissolved in DCM(1 mL), oxalyl chloride (33 μL, 0.393 mmol) was added dropwise at room temperature, and the reaction solution was spin-dried after 30 min under the protection of nitrogen after the addition, and dissolved in DCM(1 mL). The solution was dropped into a solution of isopropylamine (34 μL, 0.393 mmol) in DCM(2 mL) at room temperature. After the addition, the mixture was stirred for 10 min, washed with saturated aqueous ammonium chloride solution, and the organic phase was dried over anhydrous sodium sulfate, concentrated, and purified by silica gel column chromatography to give compound 55 (28 mg, 0.041 mmol) as a pale yellow solid, yield 52%.

ESI-MS (m/z): 678.5 [M+H]⁺, 339.9 [1/2M+H]⁺.

### Example 39 Preparation of Compound 56

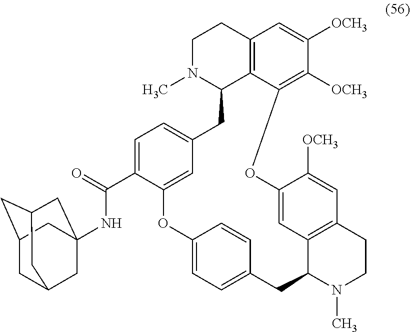 [SMILES: COc1cc2c3cc1Oc1c(OC)c(OC)cc4c1[C@@H](Cc1ccc(C(=O)NC56CC7CC(CC(C7)C5)C6)c(c1)Oc1ccc(cc1)C[C@@H]3N(C)CC2)N(C)CC4]

Compound 7 (70 mg, 0.11 mmol) was dissolved in DCM(1 mL), oxalyl chloride (49 μL, 0.55 mmol) was added dropwise at room temperature, and the reaction solution was spin-dried after 30 min under the protection of nitrogen after the addition, and dissolved in DCM(1 mL). The solution was dropped into a solution of 1-amantadine (67 mg, 0.44 mmol) and triethylamine (61 μL, 0.44 mmol) in DCM(2 mL) at room temperature. After the addition, the mixture was stirred for 30 min, washed with saturated aqueous ammonium chloride solution, and the organic phase was dried over anhydrous sodium sulfate, concentrated, and purified by silica gel column chromatography to obtain compound 56 (48 mg, 0.062 mmol) as a pale yellow solid, yield 57%.

ESI-MS (m/z): 770.7 [M+H]⁺.

### Example 40 Preparation of Compound 57

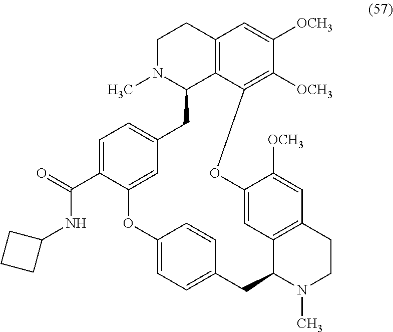 [SMILES: COc1cc2c3cc1Oc1c(OC)c(OC)cc4c1[C@@H](Cc1ccc(C(=O)NC5CCC5)c(c1)Oc1ccc(cc1)C[C@@H]3N(C)CC2)N(C)CC4]

Compound 7 (50 mg, 0.079 mmol) was dissolved in DCM(1 mL), oxalyl chloride (33 μL, 0.393 mmol) was added dropwise at room temperature, and the reaction solution was spin-dried after 30 min under the protection of nitrogen after the addition, and dissolved in DCM(1 mL) The solution was added dropwise to a solution of cyclobutylamine (34 μL, 0.393 mmol) in DCM(2 mL) at room temperature. After the addition, the mixture was stirred for 10 min, washed with saturated aqueous ammonium chloride solution, and the organic phase was dried over anhydrous sodium sulfate, concentrated, and purified by silica gel column chromatography to give compound 57 (24 mg, 0.035 mmol) as a pale yellow solid, yield 44%.

ESI-MS (m/z): 690.6 [M+H]⁺, 346.0 [1/2M+H]⁺.

### Example 41 Preparation of Compound 58

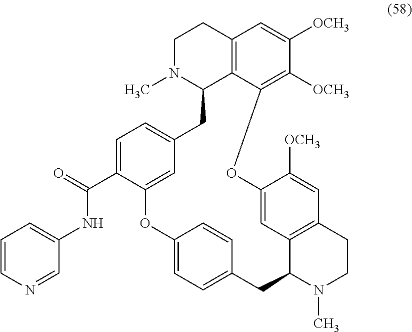 [SMILES: COc1cc2c3cc1Oc1c(OC)c(OC)cc4c1[C@@H](Cc1ccc(C(=O)Nc5cccnc5)c(c1)Oc1ccc(cc1)C[C@@H]3N(C)CC2)N(C)CC4]

Compound 7 (50 mg, 0.079 mmol) was dissolved in DCM(1 mL), oxalyl chloride (33 μL, 0.393 mmol) was added dropwise at room temperature, and the reaction solution was spin-dried after 30 min under the protection of nitrogen after the addition, and dissolved in DCM(1 mL). The solution was added dropwise to a solution of 3-aminopyridine (37 mg, 0.393 mmol) and pyridine (32 μL, 0.393 mmol) in DCM(2 mL) at room temperature. After the addition, the mixture was stirred for 10 min, washed with saturated aqueous ammonium chloride solution, and the organic phase was dried over anhydrous sodium sulfate, concentrated, and purified by silica gel column chromatography to obtain compound 58 (48 mg, 0.067 mmol) as a pale yellow solid, yield 85%.

¹H NMR (600 MHz, Chloroform-d) δ 9.86 (s, 1H), 8.62 (s, 1H), 8.32 (d, J=4.6 Hz, 1H), 8.28 (d, J=7.9 Hz, 1H), 8.12 (d, J=8.0 Hz, 1H), 7.58 (m, 1H), 7.12-7.00 (m, 3H), 6.63 (s, 1H), 6.46-6.35 (m, 2H), 6.33 (s, 1H), 5.67 (m, 1H), 4.28 (m, 1H), 3.80 (s, 3H), 3.71 (m, 1H), 3.65 (s, 3H), 3.53-3.22 (m, 3H), 3.19 (s, 3H), 3.04 (m, 2H), 2.88 (m, 2H), 2.83-2.65 (m, 5H), 2.60 (s, 3H), 2.45-2.28 (m, 3H). ESI-MS (m/z): 713.5 [M+H]⁺, 711.3 [M−H]⁻.

### Example 42 Preparation of Compound 59

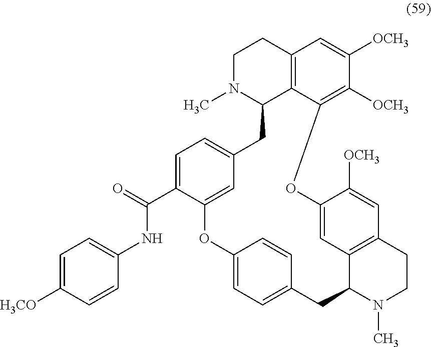 [SMILES: COc1ccc(NC(=O)c2ccc3cc2Oc2ccc(cc2)C[C@H]2c4cc(c(OC)cc4CCN2C)Oc2c(OC)c(OC)cc4c2[C@@H](C3)N(C)CC4)cc1]

Compound 7 (50 mg, 0.079 mmol) was dissolved in DCM(2 mL), oxalyl chloride (33 μL, 0.393 mmol) was added dropwise at room temperature, and the reaction solution was spin-dried after 30 min under the protection of nitrogen after the addition, and dissolved in DCM(2 mL). The solution was dropped into a solution of p-methoxyaniline (20 mg, 0.158 mmol) and pyridine (30 μL, 0.395 mmol) in DCM(2 mL) at room temperature. After the addition, the mixture was stirred for 1 h, concentrated, and purified by silica gel column chromatography to obtain compound 59 (37 mg, 0.05 mmol) as a light green solid, yield 63%.

ESI-MS (m/z): 742.6 [M+H]⁺.

### Example 43 Preparation of Compound 60

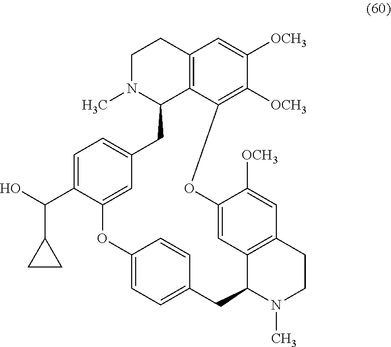 [SMILES: COc1cc2c3cc1Oc1c(OC)c(OC)cc4c1[C@@H](Cc1ccc(C(O)C5CC5)c(c1)Oc1ccc(cc1)C[C@@H]3N(C)CC2)N(C)CC4]

Compound 2 (120 mg, 0.19 mmol was dissolved in anhydrous tetrahydrofuran (1.2 ml), cooled to 0° C. in ice bath, and a solution of cyclopropylmagnesium bromide in tetrahydrofuran (0.6 mL, 0.30 mmol) was added dropwise under nitrogen protection. After the addition, the mixture was kept at this temperature for 1 h, saturated ammonium chloride was added for quenching, dichloromethane was added for extraction, and the organic phase was dried over anhydrous sodium sulfate, concentrated, and purified by silica gel column chromatography to obtain compound 60 (90 mg) as a light yellow solid, yield 70%.

ESI-MS (m/z): [M+H]⁺=663.4.

### Example 44 Preparation of Compound 61

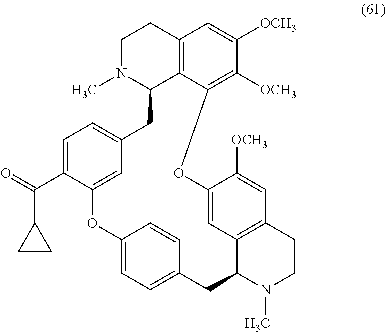 [SMILES: COc1cc2c3cc1Oc1c(OC)c(OC)cc4c1[C@@H](Cc1ccc(C(=O)C5CC5)c(c1)Oc1ccc(cc1)C[C@@H]3N(C)CC2)N(C)CC4]

Compound 60 (90 mg, 0.14 mmol) was dissolved in DCM, Dess-Martin Periodinane(86 mg, 0.20 mmol, 1.5 eq) was added at room temperature, and reacted for 1 h under nitrogen protection. The mixture was diluted with DCM, washed with sodium bicarbonate solution once, dried over anhydrous sodium sulfate, concentrated, and purified by silica gel column chromatography to obtain compound 61 (24 mg, 0.036 mmol) as a yellow solid, yield 26%,

ESI-MS (m/z): [M+H]⁺=661.4, [1/2M+H]⁺=331.4.

### Example 45 Preparation of Compound 62

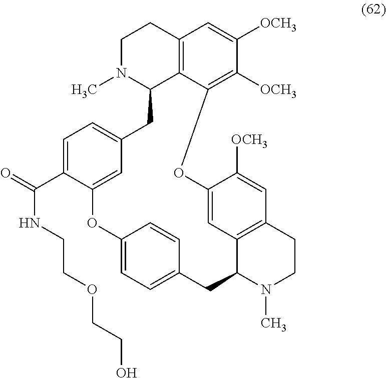 [SMILES: COc1cc2c3cc1Oc1c(OC)c(OC)cc4c1[C@@H](Cc1ccc(C(=O)NCCOCCO)c(c1)Oc1ccc(cc1)C[C@@H]3N(C)CC2)N(C)CC4]

Compound 7 (50 mg, 0.079 mmol) was dissolved in DCM(2 mL), oxalyl chloride (33 μL, 0.393 mmol) was dropped at room temperature, and the reaction solution was spin-dried after 30 min under the protection of nitrogen after the addition, and dissolved in DCM(2 mL). The solution was added dropwise to a solution of diglycolamine (42 mg, 0.395 mmol) in DCM(2 mL) at room temperature. After addition, the mixture was stirred for 30 min, concentrated, and purified by silica gel column chromatography to obtain compound 62 (39 mg, 0.054 mmol) as a pale yellow solid, yield 68%.

ESI-MS (m/z): 724.4 [M+H]⁺, 746.4 [M+Na]⁺, 362.9 [1/2M+H]⁺.

### Example 46 Preparation of Compound 63

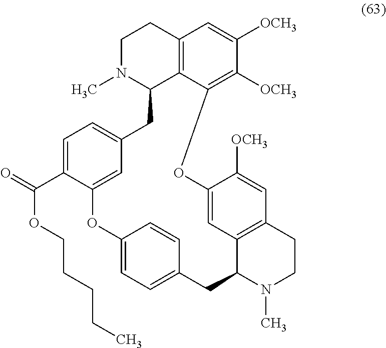 [SMILES: CCCCCOC(=O)c1ccc2cc1Oc1ccc(cc1)C[C@H]1c3cc(c(OC)cc3CCN1C)Oc1c(OC)c(OC)cc3c1[C@@H](C2)N(C)CC3]

Compound 7 (100 mg, 0.158 mmol) was suspended in n-pentanol (2 mL), concentrated sulfuric acid (65 mg, 0.662 mmol, 4.2 eq) was added at room temperature, and reacted overnight at 80° C. after addition. TLC showed raw materials were converted completely. The mixture was added with sodium bicarbonate (167 mg, 1.99 mmol, 12.6 eq), stirred for 5 min, DCM and water were added, and the organic phase was separated, dried over anhydrous sodium sulfate, concentrated, and purified by silica gel column chromatography to obtain compound 63 (79 mg, 0.112 mmol) as an off-white solid, yield 71%.

ESI-MS (m/z): 707.5 [M+H]⁺, 1412.9 [2M+H]⁺.

### Example 47 Preparation of Compound 64

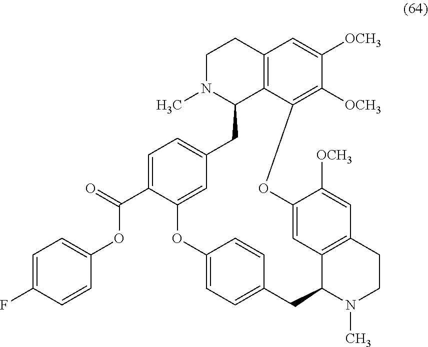 [SMILES: COc1cc2c3cc1Oc1c(OC)c(OC)cc4c1[C@@H](Cc1ccc(C(=O)Oc5ccc(F)cc5)c(c1)Oc1ccc(cc1)C[C@@H]3N(C)CC2)N(C)CC4]

Compound 7 (20 mg, 0.032 mmol) was dissolved in DCM(2 mL), oxalyl chloride (13 μL, 0.158 mmol) was added dropwise at room temperature, and the reaction solution was spin-dried after 30 min under the protection of nitrogen after the addition, and dissolved in DCM(1 mL). The solution was dropped into a solution of p-fluorophenol (18 mg, 0.158 mmol) and pyridine (12 μL, 0.158 mmol) in DCM(1 mL) at room temperature. After the addition, the mixture was stirred for 1 h, concentrated, and purified by silica gel column chromatography to obtain compound 64 (10 mg, 0.014 mmol) as a pale yellow solid, yield 43%.

ESI-MS (m/z): 731.4 [M+H]⁺.

### Example 48 Preparation of Compound 65

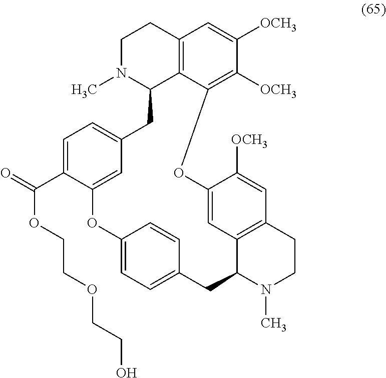 [SMILES: COc1cc2c3cc1Oc1c(OC)c(OC)cc4c1[C@@H](Cc1ccc(C(=O)OCCOCCO)c(c1)Oc1ccc(cc1)C[C@@H]3N(C)CC2)N(C)CC4]

Compound 7 (100 mg, 0.157 mmol) was mixed with diethylene glycol (1 mL), concentrated sulfuric acid (70 mg, 0.714 mmol, 4.5 eq) was added at room temperature, and reacted overnight at 80° C. after the addition. TLC showed raw materials were converted completely. After cooling to room temperature, sodium carbonate (76 mg, 0.717 mmol, 4.6 eq) was added, stirred for 5 min, DCM and water were added, and the organic phase was separated, washed with brine, dried over anhydrous sodium sulfate, concentrated, and purified by silica gel column chromatography to obtain basic compound 65 (92 mg) as an off-white solid, yield 81%.

¹H NMR (600 MHZ, Chloroform-d) δ 7.72 (d, J=8.0 Hz, 1H), 7.37 (m, 1H), 7.05 (m, 1H), 6.97 (dd, J=8.4, 2.6 Hz, 1H), 6.92 (dd, J=8.2, 2.3 Hz, 1H), 6.63 (s, 1H), 6.41 (s, 1H), 6.33 (s, 1H), 6.32 (m, 1H), 5.52 (d, J=1.5 Hz, 1H), 4.53 (m, 1H), 4.45 (m, 2H), 3.81 (s, 3H), 3.80 (m, 2H), 3.73 (m, 1H), 3.66 (m, 2H), 3.64 (s, 3H), 3.61 (m, 2H), 3.50 (m, 1H), 3.32-3.08 (m, 3H), 3.20 (s, 3H), 3.05-2.70 (m, 5H), 2.80 (s, 3H), 2.63 (s, 3H), 2.58-2.29 (m, 3H).

Hydrochloride salt as a pale yellow solid was obtained in isopropanol, ESI-MS (m/z): 725.4 [M+H]⁺, 1448.8 [2M+H]⁺.

### Example 49 Pharmacological Experiments

### 49.1 Test for Anti-Novel Coronavirus Activity

Determination for the inhibition activity of compound on 2019 novel coronavirus (SARS-CoV-2) replication: Vero E6 cells were purchased from ATCC, and SARS-CoV-2 virus was derived from the National Virus Resource Bank Microbial Virus Species Collection Center. Vero E6 cells were cultured overnight in 48-well cell culture dishes at a density of 5×10⁴ cells/well. The cells were pretreated with different concentrations of the test sample for 1 hour, and then virus (MOI of 0.01) was added to infect them for 1 hour. Then the mixture of virus and compound was taken out and the cells were further cultured with fresh medium containing the test sample. At 24 h p. i., the cell supernatant was collected and lysed in lysis buffer, and the virus copy number in the cell supernatant was quantitatively evaluated by quantitative real-time RT-PCR (qRT-PCR).

The results showed that compounds 1 to 6 significantly inhibited SARS-CoV-2 virus replication, and EC₅₀ values for the inhibition of SARS-CoV-2 virus replication were significantly better than that of berbaminehydrochloride, only about 1/10 or less of berbamine hydrochloride (Table 1). This suggests that inhibitory activity of these compounds of the present invention against coronavirus SARS-CoV-2 increases about 9.7-20 fold.

### 49.2 Cytotoxicity Test

In this example, the median toxic concentration (CC₅₀) of each example on Vero E6 cells was analysed and determined by CCK8 kit.

The results showed that the cytotoxicity of Examples 1 to 6 on Vero E6 cells was weaker or comparable to that of berbamine hydrochloride, and the therapeutic index SI was better than that of berbamine hydrochloride (Table 1).

TABLE 1
inhibitory activity of 2019 novel coronavirus (SARS-
CoV-2) replication and cytotoxicity results

| | Inhibition of | | | |
|---|---|---|---|---|
| | virus | | | |
| Compound | replication | Cytotoxicity | Therapeutic | SI |
| number | EC₅₀(μM) | CC₅₀(μM) | index SI | Ratio * |
| Compound 1 | 0.70 | 25.59 | 36.56 | 17.75 |
| Compound 2 | 1.19 | 102.00 | 85.71 | 41.61 |
| Compound 3 | 1.12 | 70.55 | 62.99 | 30.58 |
| Compound 4 | 0.94 | 31.46 | 33.47 | 16.25 |
| Compound 5 | 1.50 | 172.2 | 114.80 | 55.73 |
| Compound 8 | 0.36 | 31.4 | 87.22 | 42.34 |
| Compound 9 | 0.55 | 63.2 | 114.91 | 55.78 |
| Berbamine | 14.5 | 29.94 | 2.06 | 1.00 |
| hydrochloride | | | | |
| (Berbamine) | | | | |
| Compound 18 | 0.90 | 14.7 | 16.33 | 7.92 |
| Compound 25 | <2.0 | 29.4 | >14.7 | >7.14 |
| Compound 28 | 2.40 | 22.5 | 9.38 | 4.55 |
| Compound 33 | 0.63 | 24.3 | 38.57 | 18.72 |
| Compound 39 | 0.93 | 17.1 | 18.39 | 8.93 |
| Compound 40 | 0.95 | 11.7 | 12.31 | 5.98 |
| Compound 41 | 2.45 | 10.2 | 4.16 | 2.02 |
| Compound 42 | <2.0 | 31.1 | >15.55 | >7.55 |
| Compound 43 | 2.09 | 10.4 | 4.97 | 2.41 |
| Compound 44 | 2.25 | 10.8 | 4.80 | 2.33 |
| Compound 46 | 1.02 | 29.7 | 29.12 | 14.13 |
| Compound 47 | <2.0 | 24.6 | >12.30 | >5.97 |
| Compound 49 | <2.0 | 62.3 | >31.15 | >15.12 |
| Compound 51 | <2.0 | 10.2 | >5.10 | >2.48 |
| Compound 53 | <2.0 | 22.2 | >11.10 | >5.38 |
| Compound 54 | <2.0 | 10.5 | >5.25 | >2.55 |
| Compound 55 | <2.0 | 32 | >16 | >7.77 |
| Compound 57 | <2.0 | 33.9 | >16.95 | >8.23 |
| Compound 58 | <2.0 | 26.4 | >13.2 | >6.41 |
| Compound 59 | <2.0 | 10.6 | >5.3 | >2.57 |
| Compound 61 | <2.0 | 13.7 | >6.85 | >3.33 |
| Compound 62 | <2.0 | 122.7 | >61.35 | >29.78 |
| Compound 65 | <2.0 | 58.4 | >29.2 | >14.17 |

* SI ratio of each compound to berbamine hydrochloride.

As shown in Table 1, the SI ratio of each compound to berbamine hydrochloride is greater than 2, which indicates that the compound of the present invention not only has strong activity, but also has a large application window and significantly improved safety.

In summary, in the present invention, the inventors disclosed for the first time a novel bisbenzyl isoquinoline alkaloid or a pharmaceutically acceptable salt thereof, and a preparation method thereof, and found that it has a significant inhibitory effect on the replication of SARS-CoV-2, and inhibition EC₅₀ reaches a level of single-digit micromolar or lower. Therefore, the bisbenzyl isoquinoline alkaloid or the pharmaceutically acceptable salt thereof of the present invention has excellent anti-2019 novel coronavirus (SARS-CoV-2) 10 activity, and has a good clinical application prospect.

All literatures mentioned in the present invention are incorporated by reference herein, as though individually incorporated by reference. Additionally, it should be understood that after reading the above teaching, many variations and modifications may be made by the skilled in the art, and these equivalents also fall within the scope as defined by the appended claims.

1. A bis-benzyl isoquinoline compound shown in Formula I or a pharmaceutically acceptable salt thereof, or an enantiomer, a diastereomer, a racemate thereof, or a crystalline hydrate, a solvate thereof, or a mixture thereof, wherein, 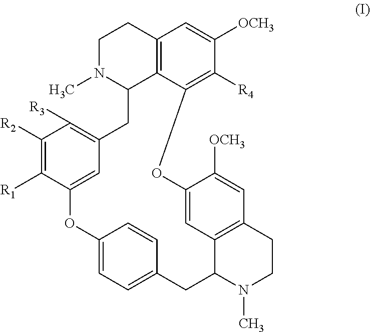
in the formula,
R₁, R₂, R₃ and R₄ are each independently selected from hydrogen, halogen, nitro, mercapto, C₁˜C₆ alkoxy, halogenated C₁˜C₆ alkoxy, hydroxy C₁˜C₆ alkoxy, C₁˜C₆ alkylthio, C₁˜C₆ alkyl, halogenated C₃˜C₆ alkyl, amino C₁˜C₆ alkyl, hydroxy C₁˜C₆ alkyl, cyano C₁˜C₆ alkyl, C₃˜C₈ cycloalkyl, C₃˜C₈ cycloalkoxy, C₂˜C₆ alkenyl, C₂˜C₆ alkenyl carbonyl, C₂˜C₆ alkenyloxy, C₂˜C₆ alkynyl, C₂˜C₆ alkynyloxy, amino, amino substituted by C₁˜C₆ alkyl, amino substituted by benzyl, amino substituted by C₁˜C₆ alkanoyl, amino substituted by C₂-C₆ alkenylacyl, cyano, C₁-C₆ carboxyl, C₁-C₆ aldehyde, C₁-C₆ alkanoyl, C₃-C₆ cycloalkyl acyl, halogenated C₁-C₆ alkanoyl, sulfonamino, sulfonamino substituted by C₁-C₆ alkyl, carbamoyl, phenyl carbamoyl, N-methyl-N-methoxyamino, carbamoyl substituted by C₁˜C₆ alkyl, carbamoyl substituted by C₃˜C₆ cycloalkyl, adamantylcarbamoyl, carbamoyl substituted by pyridinyl or pyrimidinyl, carbamoyl substituted by hydroxy C₁˜C₆ alkoxy C₁˜C₆ alkyl, C₁˜C₆ alkoxycarbonyl substituted by hydroxy C₁˜C₆ alkoxy, phenoxycarbonyl, hydroxymethyl substituted by C3˜C6 cycloalkyl, carboxyl C₁˜C₆ alkyl, C₁˜C₆ alkylsulfonyl, halogenated C₁˜C₆ alkylsulfonyl, amino C₁˜C₆ alkyl substituted by C₁˜C₆ alkyl, carbamoyloxy substituted by C₁˜C₆ alkyl, amino C₁˜C₆ alkyl substituted by C₁˜C₆ alkanoyl, C₁˜C₆ alkoxycarbonyl, carbamoyl C₁˜C₆ alkyl, carbamoyl C₁˜C₆ alkyl substituted by C₁˜C₆ alkyl, C₂˜C₆ alkenyl acyloxy (C₂˜C₆ alkenyl-CO—O—), C₂-C₁₀ ester, 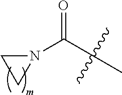
or —O—Z, wherein Z is 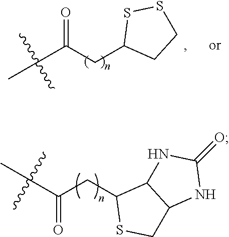
wherein n is 0, 1, 2, 3, or 4; m is 1, 2, 3, or 4;
wherein the phenylcarbamoyl, phenoxycarbonyl is optionally substituted with one or more substituents selected from F, Cl, Br, I, C₁˜C₆ alkyl, halogenated C₁˜C₆ alkyl, C₁˜C₆ alkoxy or halogenated C₁˜C₆ alkoxy;
or R₁ and R₂, or R₂ and R₃, together with the adjacent benzene ring, form benzo[5-6 membered monocyclic heterocycle] unsubstituted or substituted with 1-4 substituents, wherein the substituents are selected from halogen, hydroxy, mercapto, oxo, thio, C₁-C₆ alkyl, C₁-C₆ alkoxy, cyano; the heterocycle contains 1 to 3 heteroatoms selected from N, O, S.

2. The compound of claim 1, wherein:
R₁, R₂, R₃ and R₄ are each independently selected from hydrogen, halogen, nitro, mercapto, C₁-C₄ alkoxy, halogenated C₁-C₄ alkoxy, hydroxy C₁-C₄ alkoxy, C₁-C₄ alkylthio, C₁-C₄ alkyl, halogenated C₁-C₄ alkyl, amino C₁-C₄ alkyl, hydroxy C₁˜C₄ alkyl, cyano C₁˜C₄ alkyl, C₃˜C₆ cycloalkyl, C₃˜C₆ cycloalkoxy, C₂˜C₅ alkenyl, C₂˜C₅ alkenyl carbonyl, C₂˜C₅ alkenyloxy, C₂˜C₅ alkynyl, C₂˜C₅ alkynyloxy, amino, amino substituted by C₁˜C₄ alkyl, amino substituted by benzyl, amino substituted by C₁˜C₄ alkanoyl, amino substituted by C₂˜C₅ alkenylacyl, cyano, C₁˜C₄ carboxyl, C₁˜C₄ aldehyde, C₁˜C₄ alkanoyl, C₃˜C₅ cycloalkanoyl, halogenated C₁˜C₄ alkanoyl, sulfonylamino (—SO₂NH₂), sulfonylamino (—SO₂NH₂) substituted by C₁˜C₄ alkyl, carbamoyl (—CONH₂), phenylcarbamoyl, N-methyl-N-methoxyamino, carbamoyl substituted by C₁˜C₄ alkyl, carbamoyl substituted by C₃˜C₅ cycloalkyl, adamantyl carbamoyl, carbamoyl substituted by pyridinyl or pyrimidinyl, carbamoyl substituted by hydroxy C1˜C4 alkoxy C1˜C4 alkyl, C1˜C4 alkoxycarbonyl substituted by hydroxy C1˜C4 alkoxy, phenoxycarbonyl, hydroxymethyl substituted by C3˜C5 cycloalkyl, carboxyl C₁˜C₄ alkyl, C₁˜C₄ alkylsulfonyl, halogenated C₁˜C₄ alkylsulfonyl, amino C₁˜C₄ alkyl substituted by C₁˜C₄ alkyl, carbamoyloxy substituted by C₁˜C₄ alkyl, amino C₁˜C₄ alkyl substituted by C₁˜C₄ alkanoyl, C₁˜C₄ alkoxycarbonyl, carbamoyl C₁˜C₄ alkyl, carbamoyl C₁˜C₄ alkyl substituted by C₁˜C₄ alkyl, C₂˜C₅ alkenyl acyloxy (C₂˜C₅ alkenyl-CO—O—), C₂-C₆ ester, 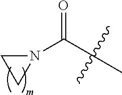
or —O—Z, wherein Z is 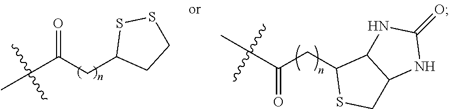
n is 0, 1, 2, 3, or 4; m is 1, 2, 3, or 4;
alternatively, R₁ and R₂, or R₂ and R₃, together with the adjacent benzene ring, may form benzo[5-6 membered monocyclic heterocycle] unsubstituted or substituted with 1 to 2 substituents;
wherein the substituent is selected from halogen, hydroxy, mercapto, oxo(=O), thio(=S), C₁-C₆ alkyl, C₁-C₆ alkoxy, cyano; wherein the heterocycle contains 1 to 3 heteroatoms selected from N, O, S.

3. The compound of claim 1, wherein:
R₁, R₂, R₃ and R₄ are each independently selected from hydrogen, fluorine, chlorine, bromine, nitro, mercapto, methoxy, ethoxy, trifluoromethoxy, —SCH₃, —SCH₂CH₃, propyl, cyclopropyl, isopropyl, tert-butyl, trifluoromethyl, difluoromethoxy, bromomethyl, chloromethyl, vinyl, vinyl methyl, amino, N-methylamino, N-ethylamino, N,N-dimethylamino, N,N-diethylamino, cyano, carboxyl, aldehyde, —CH₂NH₂, —CH₂CH₂NH₂, —CH₂OH, —CH₂CH₂OH, —CH₂CN, —CH₂CH₂CN, formyl, acetyl, propionyl, trifluoroacetyl, sulfonylamino, carbamoyl, N-methylcarbamoyl, N,N-dimethylcarbamoyl, N-ethylcarbamoyl, N,N-diethylcarbamoyl, —CH₂CO₂H, —CH₂CH₂CO₂H, —SO₂CH₃, —SO₂CF₃, —CH₂NHMe, —CH₂NMe₂, —CH₂CONH₂, —NHCOCH₃, —CH₂NHCOCH₃, —CH₂CONHMe, —CH₂CONMe₂, or —O—Z, wherein Z is 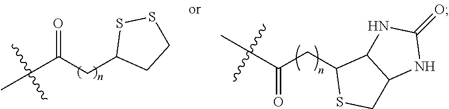
n is any integer from 0 to 4;
alternatively, R₁ and R₂, or R₂ and R₃, together with the adjacent benzene ring, may form benzo[5-6 membered monocyclic heterocycle] unsubstituted or substituted with 1-2 substituents selected from halogen, hydroxy, mercapto, oxo, thio, C₁-C₆ alkyl; the heterocycle contains 1 to 3 heteroatoms selected from N, O, S.

4. The compound of claim 1, wherein the compound of formula (I) is selected from the following compounds:

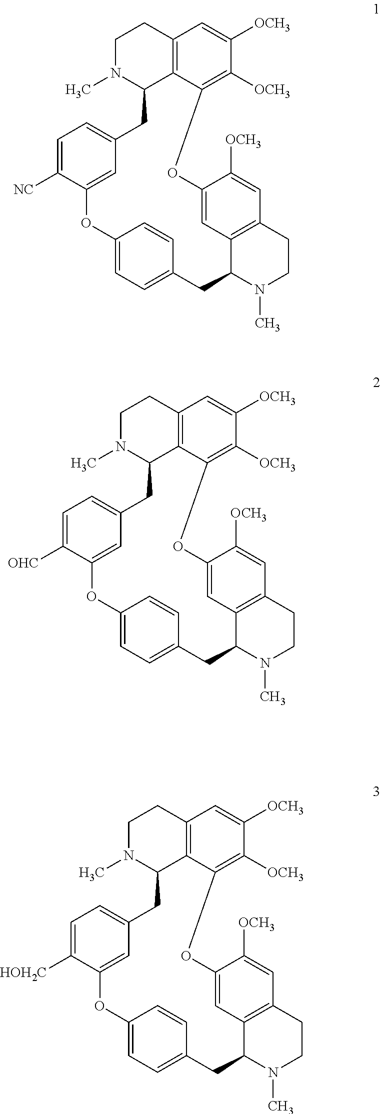 [SMILES: COc1cc2c3cc1Oc1c(OC)c(OC)cc4c1[C@@H](Cc1ccc(C#N)c(c1)Oc1ccc(cc1)C[C@@H]3N(C)CC2)N(C)CC4 ; COc1cc2c3cc1Oc1c(OC)c(OC)cc4c1[C@@H](Cc1ccc(C=O)c(c1)Oc1ccc(cc1)C[C@@H]3N(C)CC2)N(C)CC4 ; COc1cc2c3cc1Oc1c(OC)c(OC)cc4c1[C@@H](Cc1ccc(CO)c(c1)Oc1ccc(cc1)C[C@@H]3N(C)CC2)N(C)CC4]

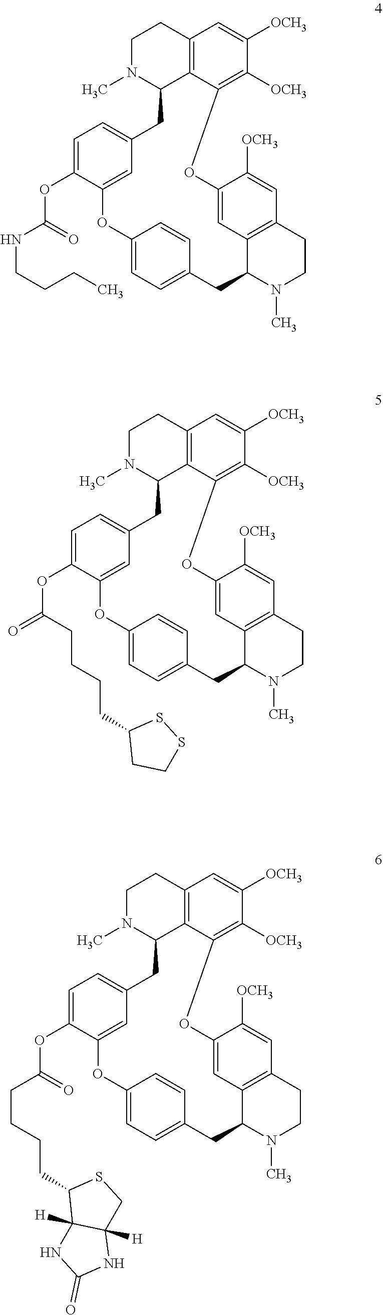 [SMILES: CCCCNC(=O)Oc1ccc2cc1Oc1ccc(cc1)C[C@H]1c3cc(c(OC)cc3CCN1C)Oc1c(OC)c(OC)cc3c1[C@@H](C2)N(C)CC3 ; COc1cc2c3cc1Oc1c(OC)c(OC)cc4c1[C@@H](Cc1ccc(OC(=O)CCCC[C@H]5CCSS5)c(c1)Oc1ccc(cc1)C[C@@H]3N(C)CC2)N(C)CC4 ; COc1cc2c3cc1Oc1c(OC)c(OC)cc4c1[C@@H](Cc1ccc(OC(=O)CCCC[C@@H]5SC[C@@H]6NC(=O)N[C@@H]65)c(c1)Oc1ccc(cc1)C[C@@H]3N(C)CC2)N(C)CC4]

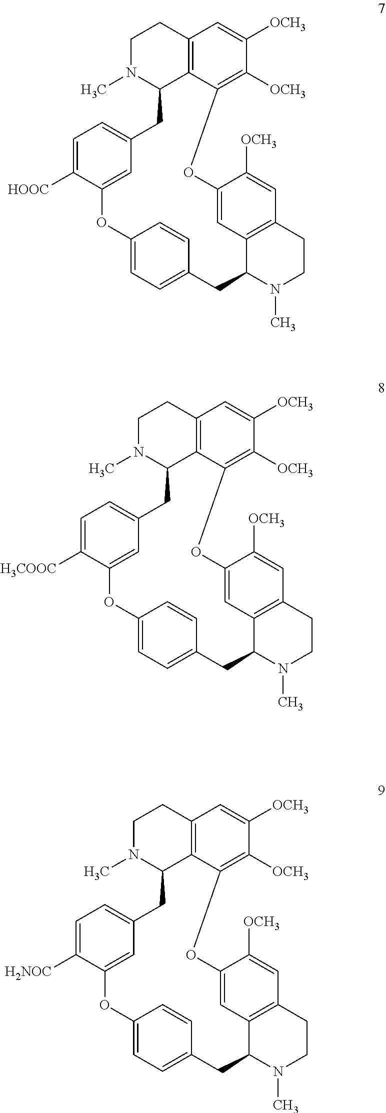

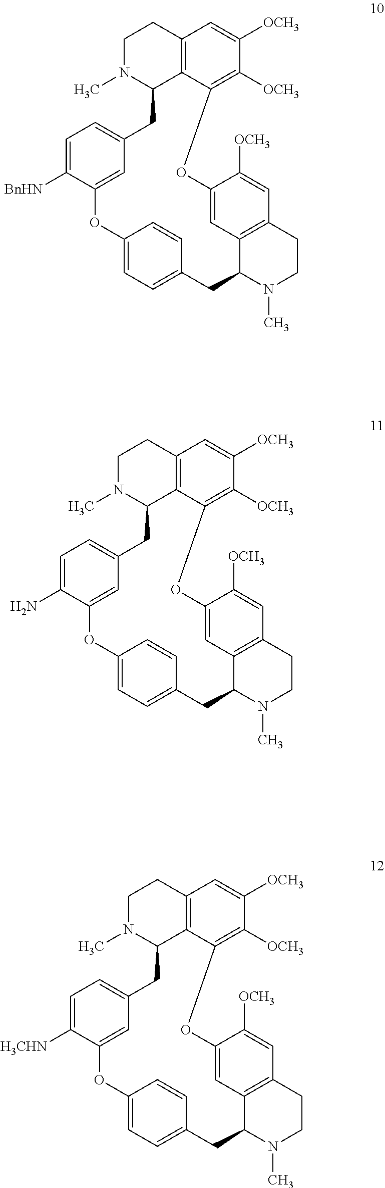

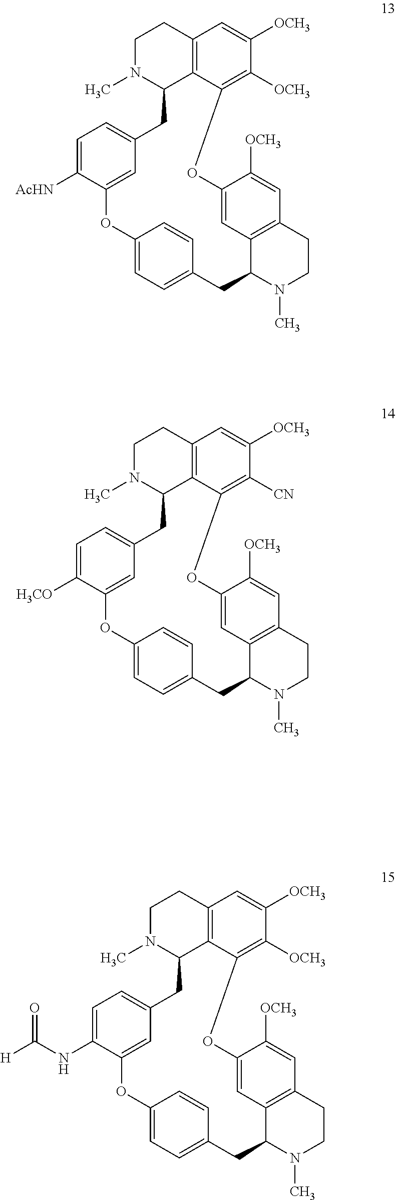 [SMILES: COc1cc2c3cc1Oc1c(OC)c(OC)cc4c1[C@@H](Cc1ccc(NC(C)=O)c(c1)Oc1ccc(cc1)C[C@@H]3N(C)CC2)N(C)CC4 ; COc1ccc2cc1Oc1ccc(cc1)C[C@H]1c3cc(c(OC)cc3CCN1C)Oc1c(C#N)c(OC)cc3c1[C@@H](C2)N(C)CC3 ; COc1cc2c3cc1Oc1c(OC)c(OC)cc4c1[C@@H](Cc1ccc(NC=O)c(c1)Oc1ccc(cc1)C[C@@H]3N(C)CC2)N(C)CC4]

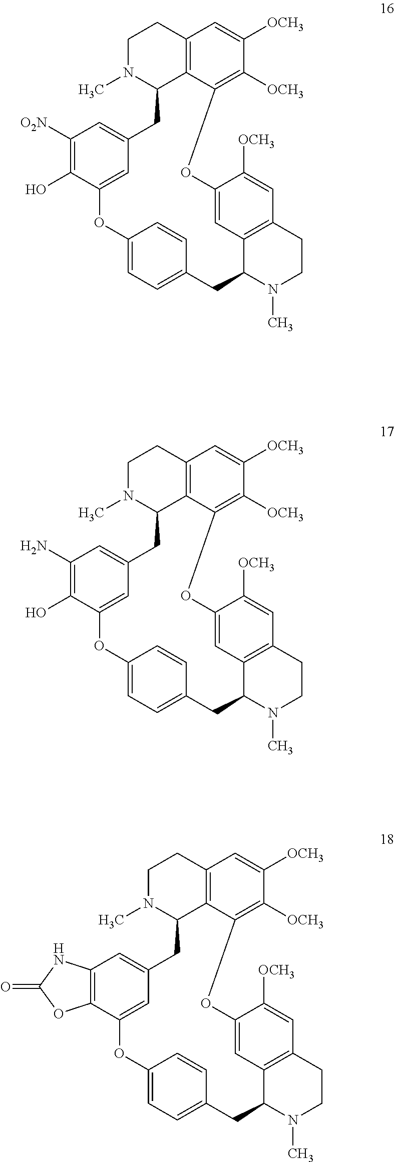 [SMILES: COc1cc2c3cc1Oc1c(OC)c(OC)cc4c1[C@@H](Cc1cc(c(O)c([N+](=O)[O-])c1)Oc1ccc(cc1)C[C@@H]3N(C)CC2)N(C)CC4 ; COc1cc2c3cc1Oc1c(OC)c(OC)cc4c1[C@@H](Cc1cc(N)c(O)c(c1)Oc1ccc(cc1)C[C@@H]3N(C)CC2)N(C)CC4 ; COc1cc2c3cc1Oc1c(OC)c(OC)cc4c1[C@@H](Cc1cc(c5oc(=O)[nH]c5c1)Oc1ccc(cc1)C[C@@H]3N(C)CC2)N(C)CC4]

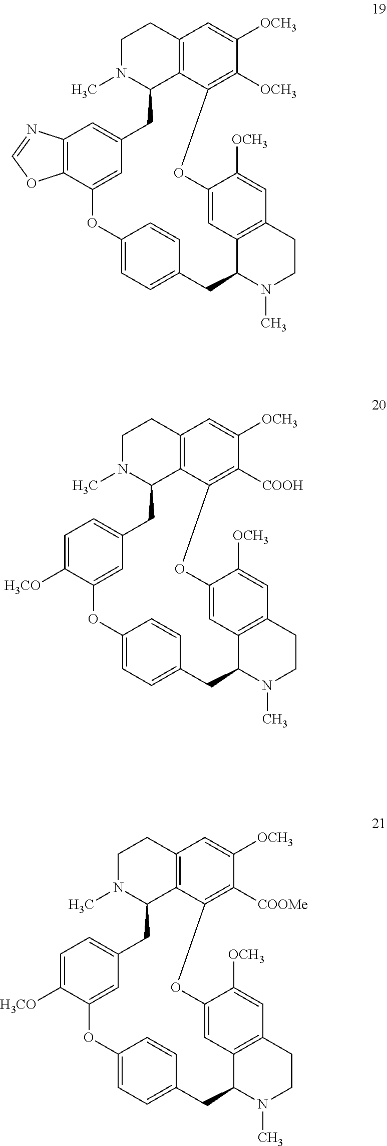 [SMILES: COc1cc2c3cc1Oc1c(OC)c(OC)cc4c1[C@@H](Cc1cc(c5ocnc5c1)Oc1ccc(cc1)C[C@@H]3N(C)CC2)N(C)CC4 ; COc1ccc2cc1Oc1ccc(cc1)C[C@H]1c3cc(c(OC)cc3CCN1C)Oc1c(C(=O)O)c(OC)cc3c1[C@@H](C2)N(C)CC3 ; COC(=O)c1c(OC)cc2c3c1Oc1cc4c(cc1OC)CCN(C)[C@H]4Cc1ccc(cc1)Oc1cc(ccc1OC)C[C@H]3N(C)CC2]

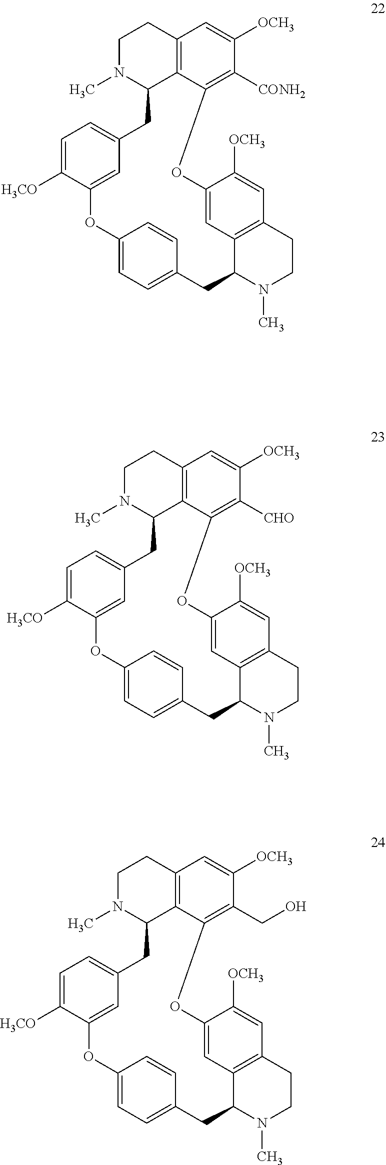 [SMILES: COc1ccc2cc1Oc1ccc(cc1)C[C@H]1c3cc(c(OC)cc3CCN1C)Oc1c(C(N)=O)c(OC)cc3c1[C@@H](C2)N(C)CC3 ; COc1ccc2cc1Oc1ccc(cc1)C[C@H]1c3cc(c(OC)cc3CCN1C)Oc1c(C=O)c(OC)cc3c1[C@@H](C2)N(C)CC3 ; COc1ccc2cc1Oc1ccc(cc1)C[C@H]1c3cc(c(OC)cc3CCN1C)Oc1c(CO)c(OC)cc3c1[C@@H](C2)N(C)CC3]

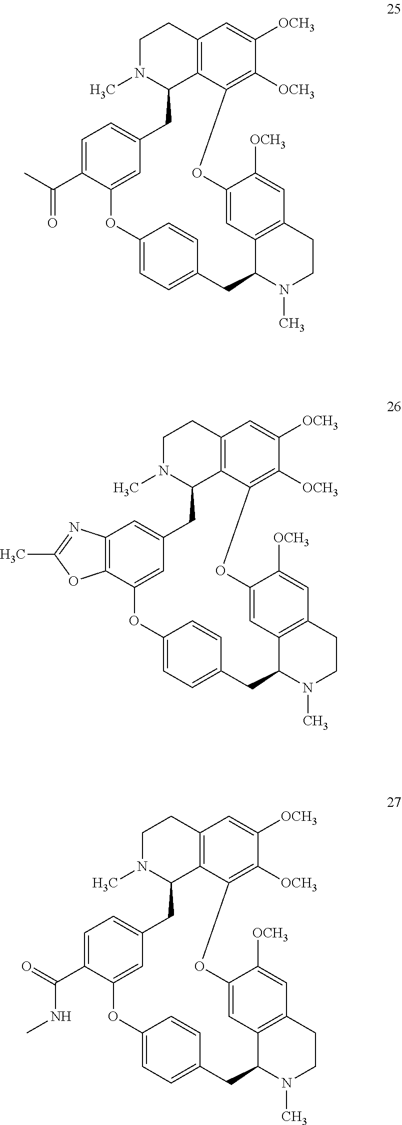 [SMILES: COc1cc2c3cc1Oc1c(OC)c(OC)cc4c1[C@@H](Cc1ccc(C(C)=O)c(c1)Oc1ccc(cc1)C[C@@H]3N(C)CC2)N(C)CC4 ; COc1cc2c3cc1Oc1c(OC)c(OC)cc4c1[C@@H](Cc1cc(c5oc(C)nc5c1)Oc1ccc(cc1)C[C@@H]3N(C)CC2)N(C)CC4 ; CNC(=O)c1ccc2cc1Oc1ccc(cc1)C[C@H]1c3cc(c(OC)cc3CCN1C)Oc1c(OC)c(OC)cc3c1[C@@H](C2)N(C)CC3]

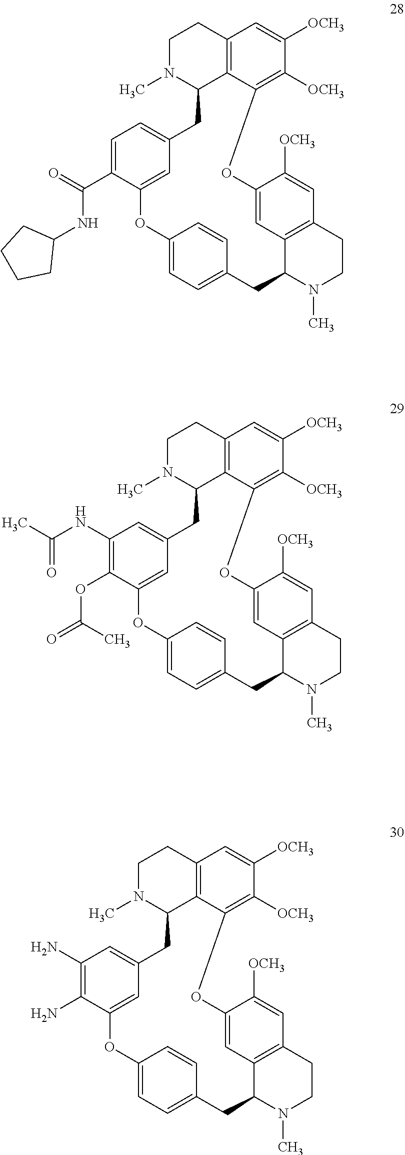 [SMILES: COc1cc2c3cc1Oc1c(OC)c(OC)cc4c1[C@@H](Cc1ccc(C(=O)NC5CCCC5)c(c1)Oc1ccc(cc1)C[C@@H]3N(C)CC2)N(C)CC4 ; COc1cc2c3cc1Oc1c(OC)c(OC)cc4c1[C@@H](Cc1cc(NC(C)=O)c(OC(C)=O)c(c1)Oc1ccc(cc1)C[C@@H]3N(C)CC2)N(C)CC4 ; COc1cc2c3cc1Oc1c(OC)c(OC)cc4c1[C@@H](Cc1cc(N)c(N)c(c1)Oc1ccc(cc1)C[C@@H]3N(C)CC2)N(C)CC4]

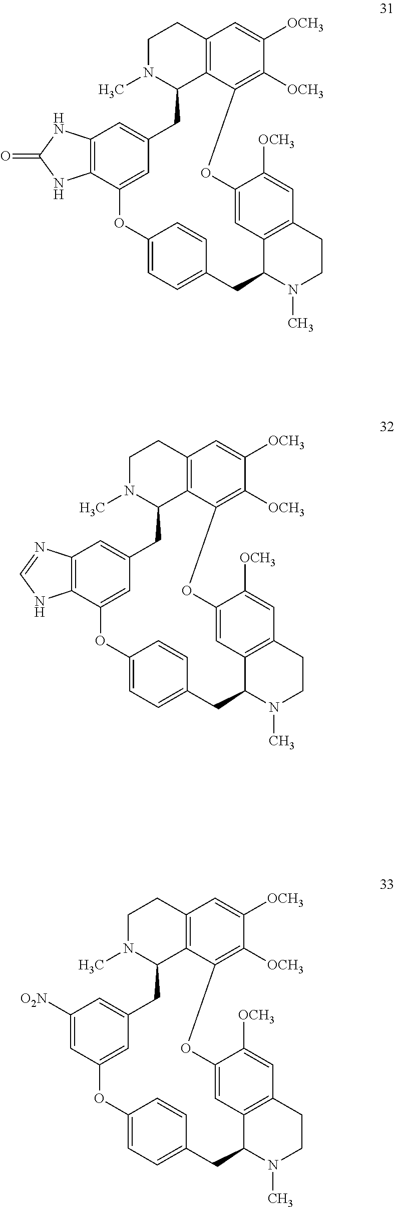 [SMILES: COc1cc2c3cc1Oc1c(OC)c(OC)cc4c1[C@@H](Cc1cc(c5[nH]c(=O)[nH]c5c1)Oc1ccc(cc1)C[C@@H]3N(C)CC2)N(C)CC4 ; COc1cc2c3cc1Oc1c(OC)c(OC)cc4c1[C@@H](Cc1cc(c5[nH]cnc5c1)Oc1ccc(cc1)C[C@@H]3N(C)CC2)N(C)CC4 ; COc1cc2c3cc1Oc1c(OC)c(OC)cc4c1[C@@H](Cc1cc(cc([N+](=O)[O-])c1)Oc1ccc(cc1)C[C@@H]3N(C)CC2)N(C)CC4]

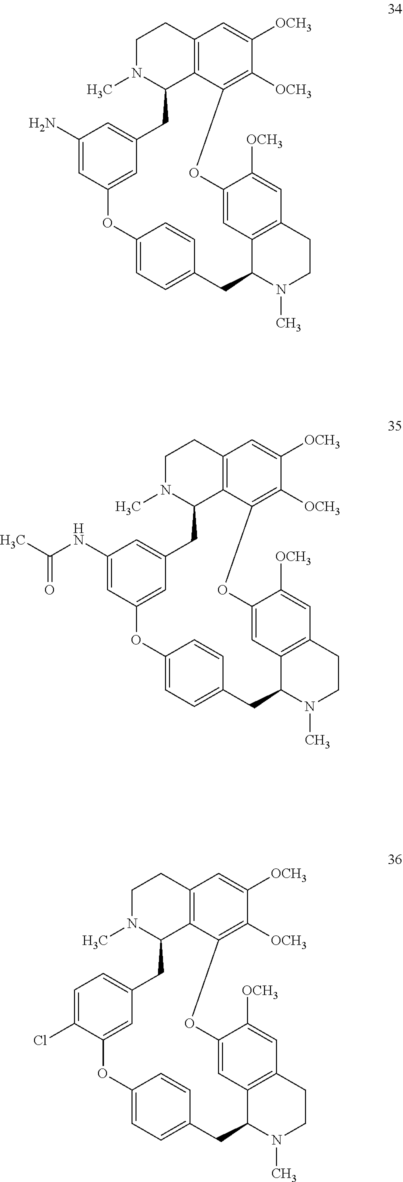 [SMILES: COc1cc2c3cc1Oc1c(OC)c(OC)cc4c1[C@@H](Cc1cc(N)cc(c1)Oc1ccc(cc1)C[C@@H]3N(C)CC2)N(C)CC4 ; COc1cc2c3cc1Oc1c(OC)c(OC)cc4c1[C@@H](Cc1cc(NC(C)=O)cc(c1)Oc1ccc(cc1)C[C@@H]3N(C)CC2)N(C)CC4 ; COc1cc2c3cc1Oc1c(OC)c(OC)cc4c1[C@@H](Cc1ccc(Cl)c(c1)Oc1ccc(cc1)C[C@@H]3N(C)CC2)N(C)CC4]

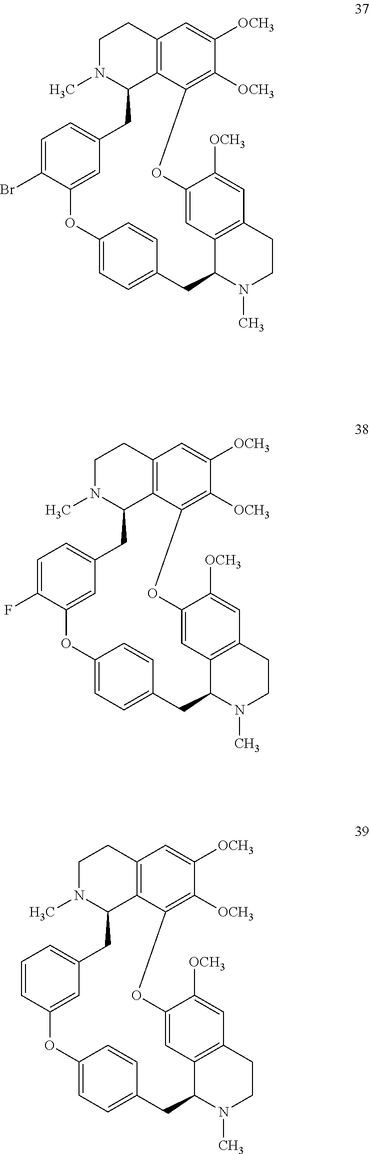 [SMILES: COc1cc2c3cc1Oc1c(OC)c(OC)cc4c1[C@@H](Cc1ccc(Br)c(c1)Oc1ccc(cc1)C[C@@H]3N(C)CC2)N(C)CC4 ; COc1cc2c3cc1Oc1c(OC)c(OC)cc4c1[C@@H](Cc1ccc(F)c(c1)Oc1ccc(cc1)C[C@@H]3N(C)CC2)N(C)CC4 ; COc1cc2c3cc1Oc1c(OC)c(OC)cc4c1[C@@H](Cc1cccc(c1)Oc1ccc(cc1)C[C@@H]3N(C)CC2)N(C)CC4]

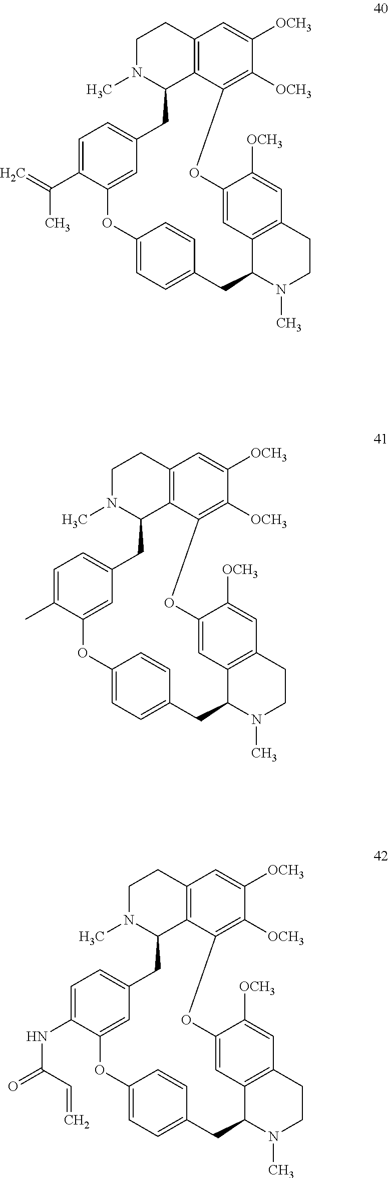 [SMILES: C=C(C)c1ccc2cc1Oc1ccc(cc1)C[C@H]1c3cc(c(OC)cc3CCN1C)Oc1c(OC)c(OC)cc3c1[C@@H](C2)N(C)CC3 ; COc1cc2c3cc1Oc1c(OC)c(OC)cc4c1[C@@H](Cc1ccc(C)c(c1)Oc1ccc(cc1)C[C@@H]3N(C)CC2)N(C)CC4 ; C=CC(=O)Nc1ccc2cc1Oc1ccc(cc1)C[C@H]1c3cc(c(OC)cc3CCN1C)Oc1c(OC)c(OC)cc3c1[C@@H](C2)N(C)CC3]

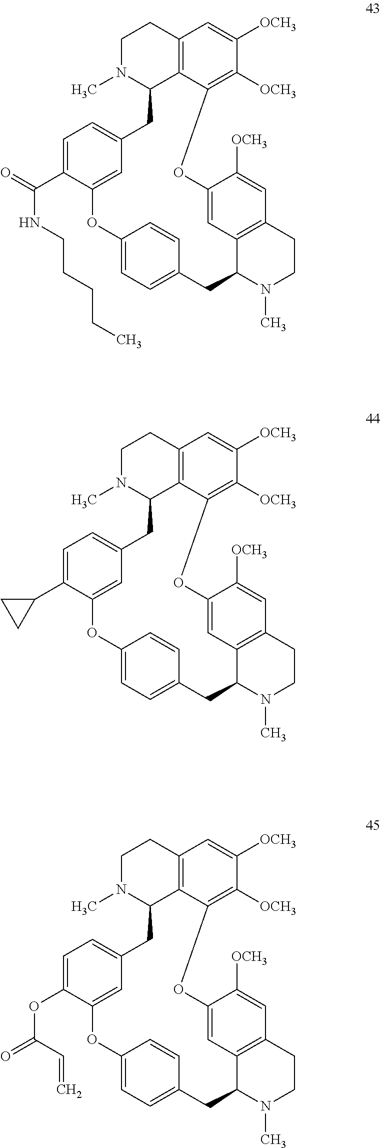 [SMILES: CCCCCNC(=O)c1ccc2cc1Oc1ccc(cc1)C[C@H]1c3cc(c(OC)cc3CCN1C)Oc1c(OC)c(OC)cc3c1[C@@H](C2)N(C)CC3 ; COc1cc2c3cc1Oc1c(OC)c(OC)cc4c1[C@@H](Cc1ccc(C5CC5)c(c1)Oc1ccc(cc1)C[C@@H]3N(C)CC2)N(C)CC4 ; C=CC(=O)Oc1ccc2cc1Oc1ccc(cc1)C[C@H]1c3cc(c(OC)cc3CCN1C)Oc1c(OC)c(OC)cc3c1[C@@H](C2)N(C)CC3]

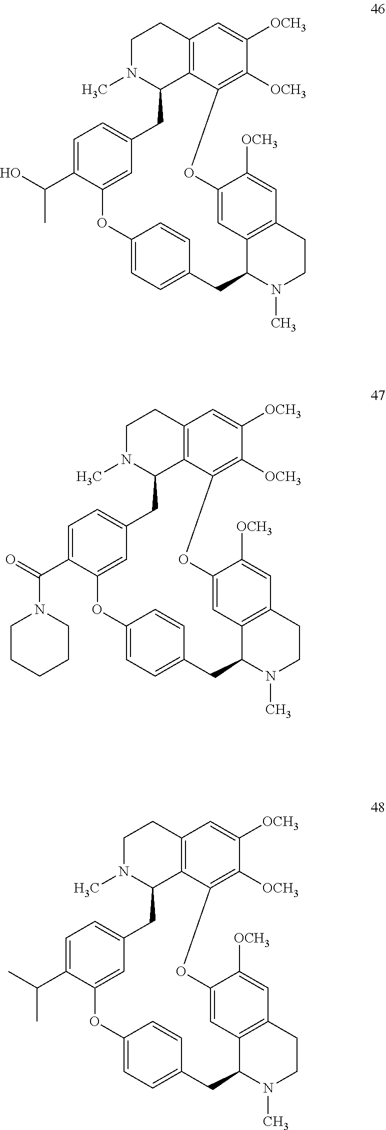 [SMILES: COc1cc2c3cc1Oc1c(OC)c(OC)cc4c1[C@@H](Cc1ccc(C(C)O)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)N5CCCCC5)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(C)C)c(c1)Oc1ccc(cc1)C[C@@H]3N(C)CC2)N(C)CC4]

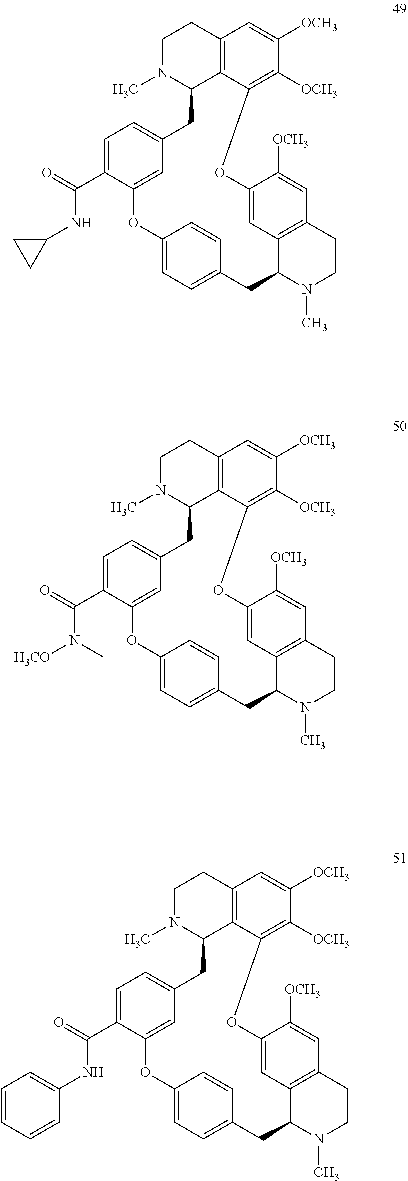 [SMILES: COc1cc2c3cc1Oc1c(OC)c(OC)cc4c1[C@@H](Cc1ccc(C(=O)NC5CC5)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)N(C)OC)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)Nc5ccccc5)c(c1)Oc1ccc(cc1)C[C@@H]3N(C)CC2)N(C)CC4]

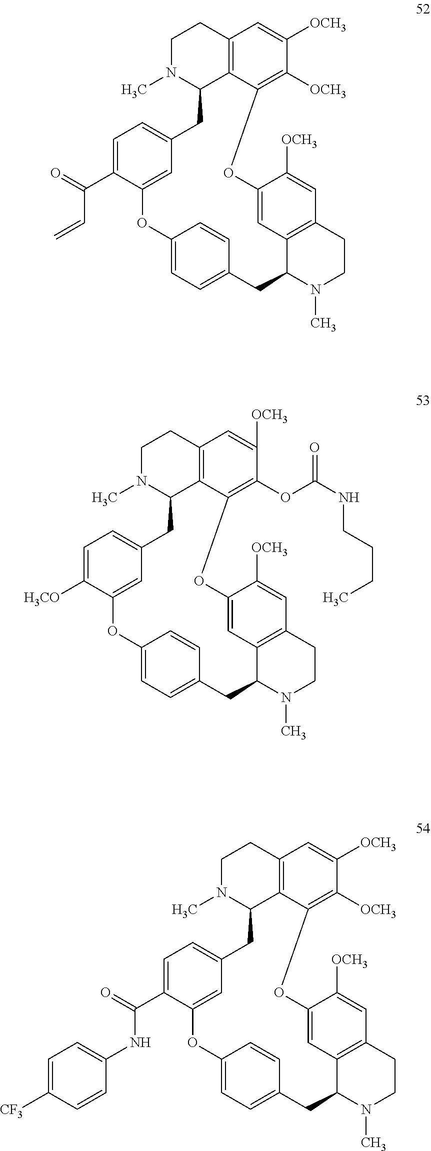 [SMILES: C=CC(=O)c1ccc2cc1Oc1ccc(cc1)C[C@H]1c3cc(c(OC)cc3CCN1C)Oc1c(OC)c(OC)cc3c1[C@@H](C2)N(C)CC3 ; CCCCNC(=O)Oc1c(OC)cc2c3c1Oc1cc4c(cc1OC)CCN(C)[C@H]4Cc1ccc(cc1)Oc1cc(ccc1OC)C[C@H]3N(C)CC2 ; COc1cc2c3cc1Oc1c(OC)c(OC)cc4c1[C@@H](Cc1ccc(C(=O)Nc5ccc(C(F)(F)F)cc5)c(c1)Oc1ccc(cc1)C[C@@H]3N(C)CC2)N(C)CC4]

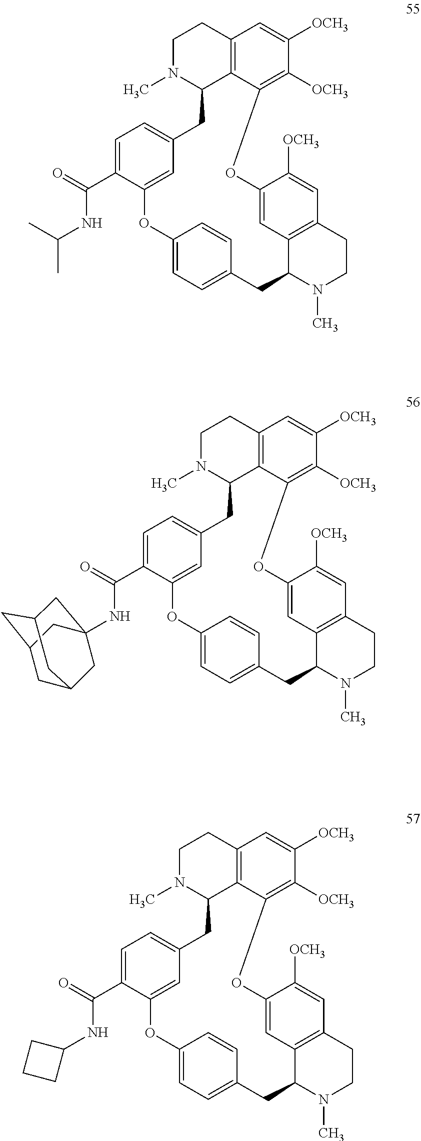 [SMILES: COc1cc2c3cc1Oc1c(OC)c(OC)cc4c1[C@@H](Cc1ccc(C(=O)NC(C)C)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)NC56CC7CC(CC(C7)C5)C6)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)NC5CCC5)c(c1)Oc1ccc(cc1)C[C@@H]3N(C)CC2)N(C)CC4]

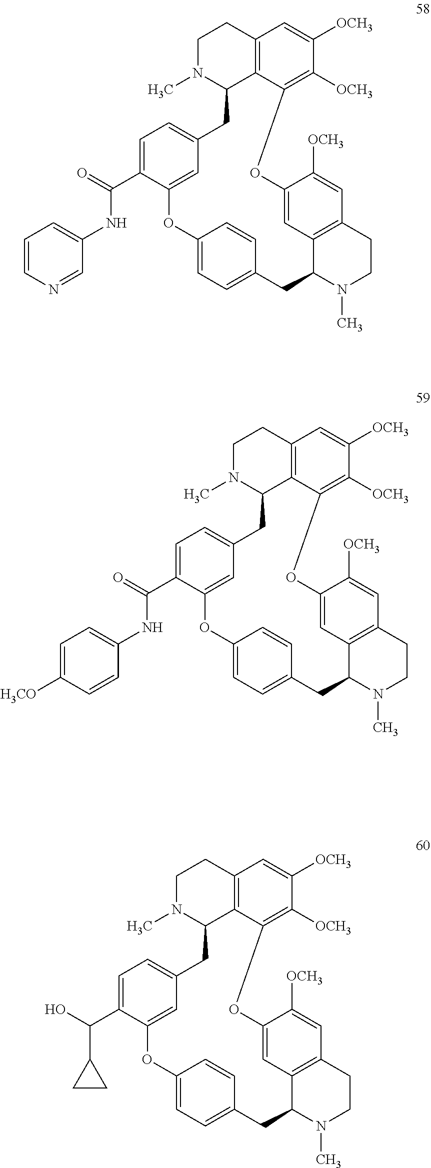 [SMILES: COc1cc2c3cc1Oc1c(OC)c(OC)cc4c1[C@@H](Cc1ccc(C(=O)Nc5cccnc5)c(c1)Oc1ccc(cc1)C[C@@H]3N(C)CC2)N(C)CC4 ; COc1ccc(NC(=O)c2ccc3cc2Oc2ccc(cc2)C[C@H]2c4cc(c(OC)cc4CCN2C)Oc2c(OC)c(OC)cc4c2[C@@H](C3)N(C)CC4)cc1 ; COc1cc2c3cc1Oc1c(OC)c(OC)cc4c1[C@@H](Cc1ccc(C(O)C5CC5)c(c1)Oc1ccc(cc1)C[C@@H]3N(C)CC2)N(C)CC4]

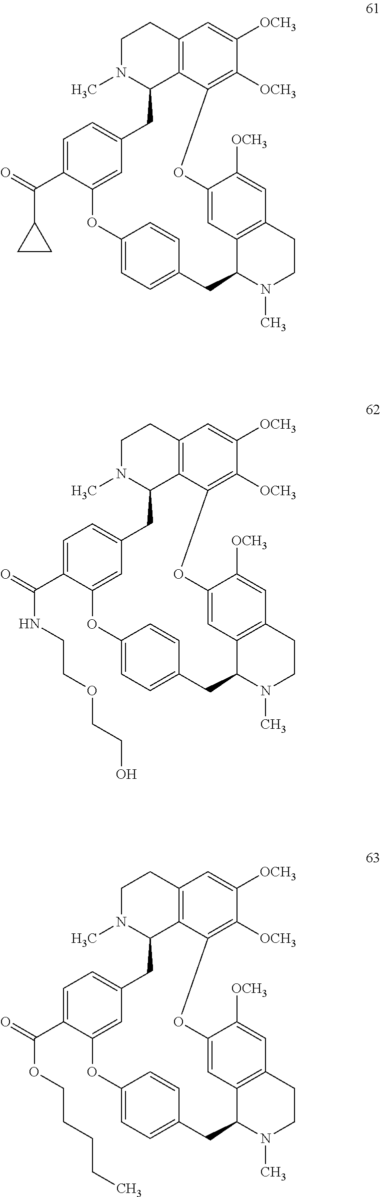 [SMILES: COc1cc2c3cc1Oc1c(OC)c(OC)cc4c1[C@@H](Cc1ccc(C(=O)C5CC5)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)NCCOCCO)c(c1)Oc1ccc(cc1)C[C@@H]3N(C)CC2)N(C)CC4 ; CCCCCOC(=O)c1ccc2cc1Oc1ccc(cc1)C[C@H]1c3cc(c(OC)cc3CCN1C)Oc1c(OC)c(OC)cc3c1[C@@H](C2)N(C)CC3]

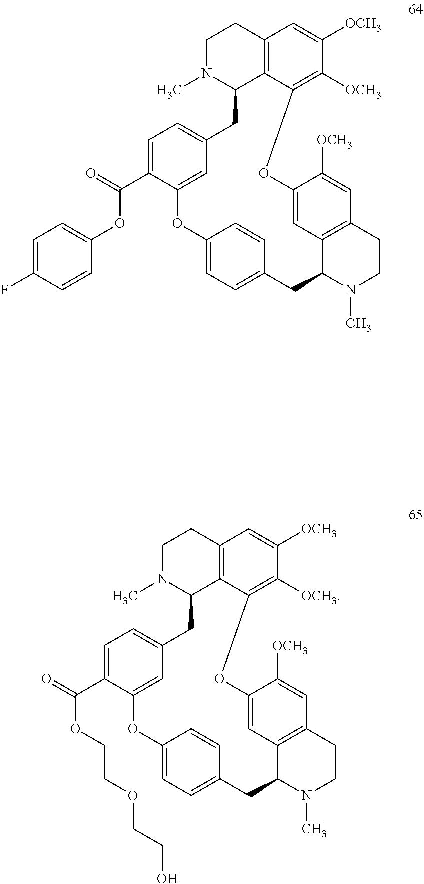 [SMILES: COc1cc2c3cc1Oc1c(OC)c(OC)cc4c1[C@@H](Cc1ccc(C(=O)Oc5ccc(F)cc5)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)OCCOCCO)c(c1)Oc1ccc(cc1)C[C@@H]3N(C)CC2)N(C)CC4]

5. The compound of claim 1, wherein the compound of formula (I) is selected from the following compounds:

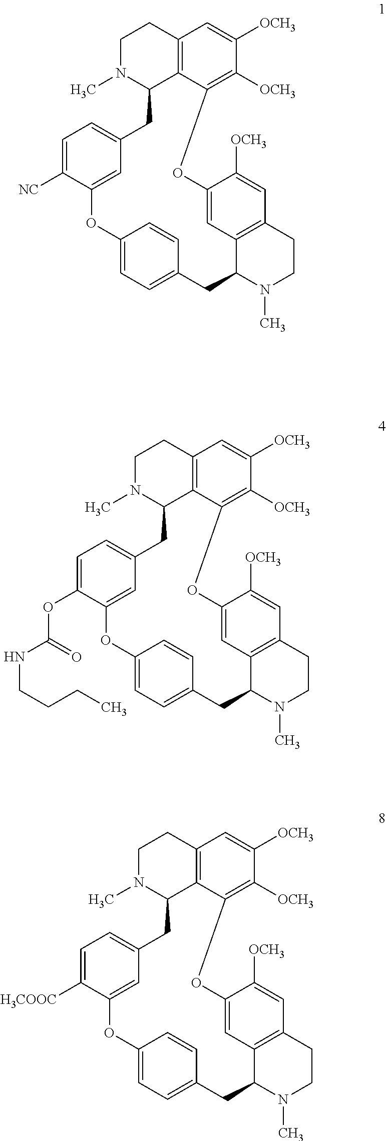

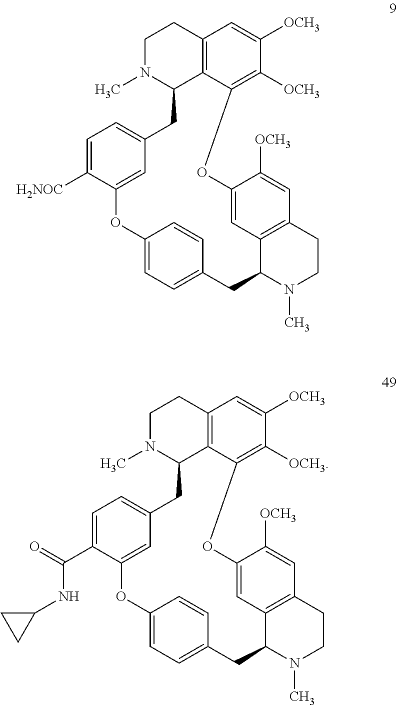 [SMILES: COc1cc2c3cc1Oc1c(OC)c(OC)cc4c1[C@@H](Cc1ccc(C(N)=O)c(c1)Oc1ccc(cc1)C[C@@H]3N(C)CC2)N(C)CC4 ; COc1cc2c3cc1Oc1c(OC)c(OC)cc4c1[C@@H](Cc1ccc(C(=O)NC5CC5)c(c1)Oc1ccc(cc1)C[C@@H]3N(C)CC2)N(C)CC4]

6. A method for preparing the bis-benzyl isoquinoline compound of claim 1, wherein the method is selected from the group consisting of:
a) the bis-benzyl isoquinoline compound is obtained by alkylation reaction between a phenolic hydroxy-containing bis-benzyl isoquinoline as a raw material and an alkylation reagent;
b) the bis-benzyl isoquinoline compound is obtained by an acylation reaction between a phenolic hydroxy-containing bis-benzyl isoquinoline as a raw material and an acylation reagent;
c) compound (I-1b) is obtained by reacting berbamine as a raw material with sulfonylation reagent in the presence of a base;
the bis-benzyl isoquinoline compound I-1 is obtained by coupling (I-1b) and coupling reagents, and the reaction is shown in scheme 1: 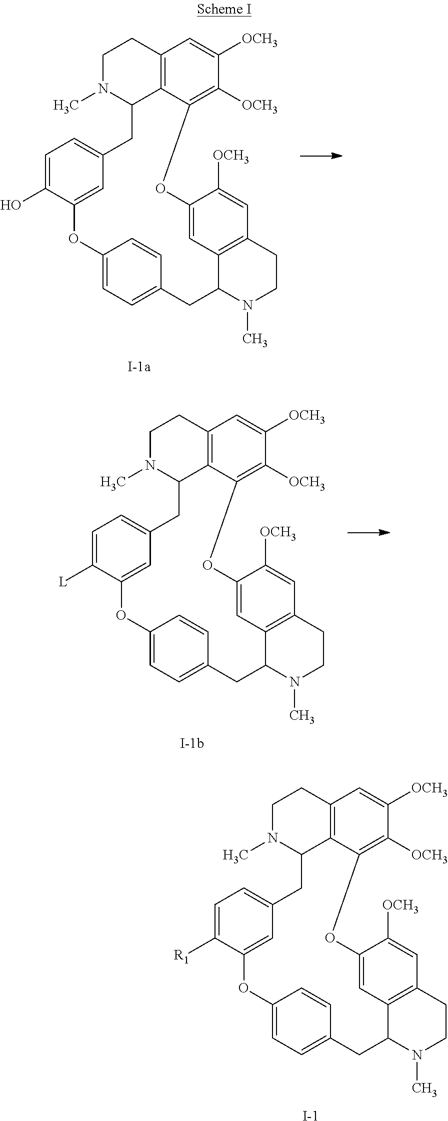
in formula I-1, R₁ is selected from hydrogen, halogen, C₁-C₆ alkylthio, C₁-C₆ alkyl, C₃-C₆ cycloalkyl, C₂-C₆ alkenyl, C₂-C₆ alkynyl, amino substituted by C₁-C₆ alkyl, amino substituted by benzyl, cyano, carboxy, aldehyde, C₁-C₆ alkanoyl;
L in formula 1-1b is selected from a leaving group;
d) using berbamine as a raw material, the compound of formula (I-2) is prepared by nitration, reduction and ring-closing reaction, as shown in the following scheme 2: 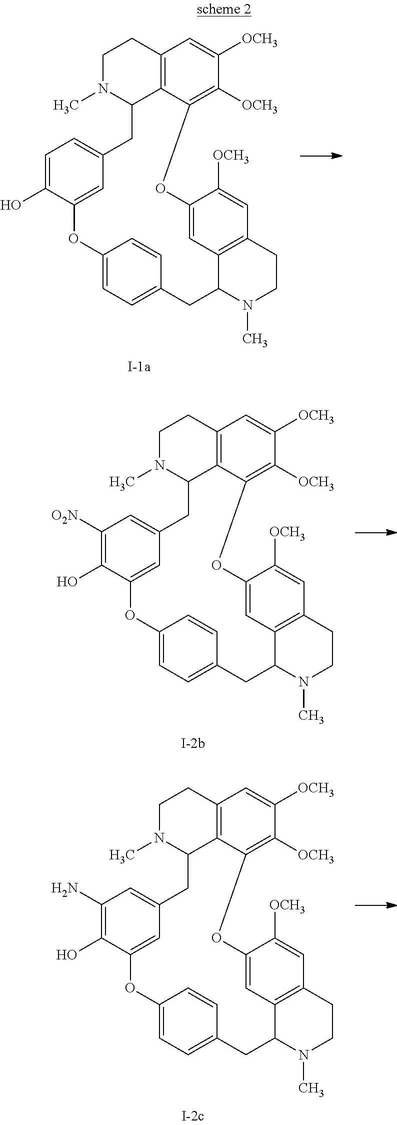 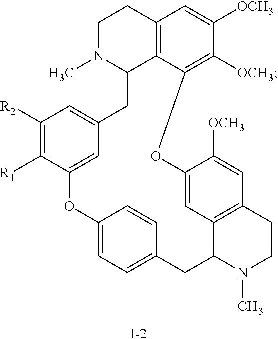
in formula I-2, R₁ and R₂, together with the adjacent benzene ring, form benzo[5-6 membered monocyclic heterocycle] unsubstituted or substituted with 1-4 substituents;
e) using berbamine as a raw material, the compound of formula (I-3) is prepared by multi-step reaction, as shown in scheme 3: 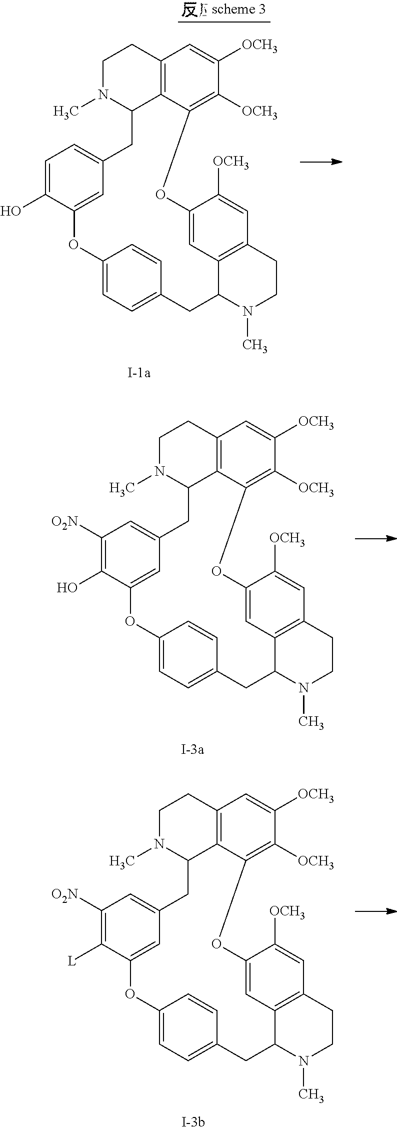 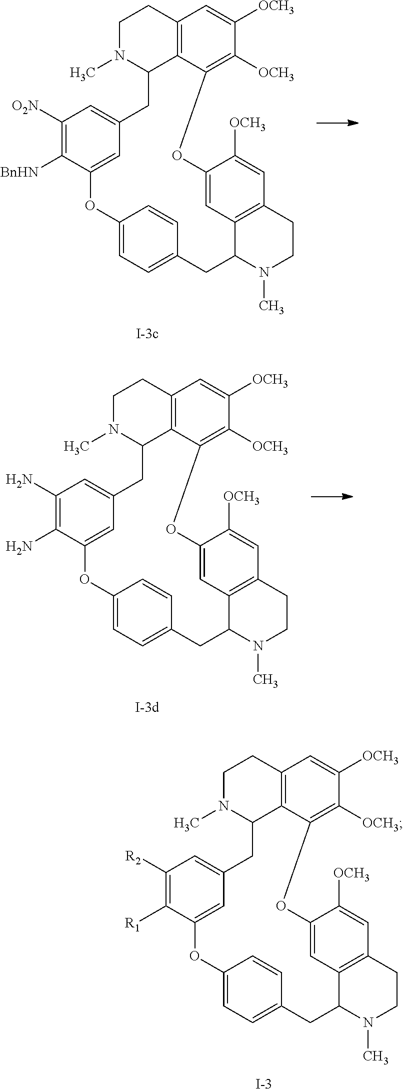
in formula I-3, R₁ and R₂ together with the adjacent benzene ring form benzo[5-6 membered monocyclic heterocycle] unsubstituted or substituted with 1-4 substituents;
f) using Fangchinoline as a raw material, reacting with sulfonylation reagent in the presence of base to obtain compound (I-4b); and conducting a coupling reaction between compound (I-4b) and coupling reagent to obtain a compound of formula I-4, as shown in scheme 4: 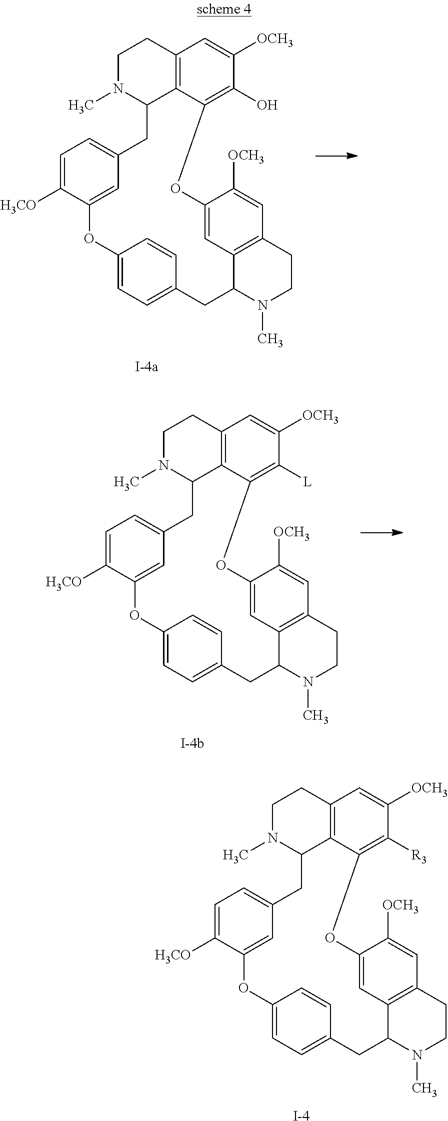
in formula I-4, R₃ is selected from hydrogen, halogen, C₁-C₆ alkylthio, C₁-C₆ alkyl, C₃-C₆ cycloalkyl, C₂-C₆ alkenyl, C₂-C₆ alkynyl, amino substituted by C₁-C₆ alkyl, amino substituted by benzyl, cyano, carboxy, aldehyde, C₁-C₆ alkanoyl;
L in formula I-4b is a leaving group;
g) the compounds obtained by methods a) to f) are subjected to functional group transformation to obtain the bis-benzyl isoquinoline compounds.

7. A pharmaceutical composition comprising:
(A1) a first active ingredient, comprising a therapeutically effective amount of one or more of the bis-benzyl isoquinoline compound of formula I of claim 1, an enantiomer, a diastereomer, a racemate, a pharmaceutically acceptable salt, a crystalline hydrate, and a solvate thereof; and
(B) a pharmaceutically acceptable carrier.

8. A method of preventing and/or treating related diseases caused by virus infection, the method comprising using the pharmaceutical composition of claim 7.

9. The method of claim 8, wherein the related disease is a coronavirus selected from the group consisting of a coronavirus infecting a human, a severe acute respiratory syndrome coronavirus (SARS-CoV), a 2019 novel coronavirus (2019-nCoV or SARS-CoV-2), a Middle East respiratory syndrome coronavirus (MERS-CoV), a coronavirus causing common cold, and a combination thereof.

10. The method of claim 8, wherein the related disease is caused by coronavirus and is selected from the group consisting of common cold caused by human coronavirus, infection with high-risk symptoms, respiratory tract infection, pneumonia and its complications, novel coronavirus pneumonia caused by SARS-CoV-2 (Corona Virus Disease 2019, COVID-19) and a combination thereof.

11. A method of preparing an inhibitor or a medicament comprising using the bis-benzyl isoquinoline compound according to claim 1, (a) the inhibitor is for inhibiting the replication of the 2019 novel coronavirus (SARS-CoV-2); and/or (b) the medicament is for treating and/or preventing or alleviating a related disease caused by the infection of 2019 novel coronavirus (SARS-CoV-2).
